(19) United States
(12) Patent Application Publication
(10) Pub. No.: US 2023/0074121 A1
(43) Pub. Date: Mar. 9, 2023

(54) ANTIBODY PANEL

(71) Applicant: AUCKLAND UNISERVICES LIMITED, Auckland (NZ)

(72) Inventors: Peter Roderick Dunbar, Auckland (NZ); Saemmul Park, Auckland (NZ)

(21) Appl. No.: 17/901,315

(22) Filed: Sep. 1, 2022

(30) Foreign Application Priority Data
Sep. 2, 2021 (AU) 2021902857

(51) Int. Cl. G01N 33/533; G01N 21/64; G01N 33/577

(52) U.S. Cl. CPC G01N 33/533; G01N 21/6428; G01N 33/577; G01N 2021/6421; G01N 2021/6441; G01N 1/30

(57) ABSTRACT

Disclosed herein are certain sets of antibody-fluorophore pairs comprising antibodies that specifically bind target antigens, the sets of antibody-fluorophore pairs thereby allowing visualization and quantification of a plurality of target antigens in a biological sample using multispectral tissue slide scanners. Also disclosed herein are methods of using such antibody-fluorophore pairs, and reagents related thereto.

### RELATED APPLICATIONS

The present patent application claims the priority benefit of Australian Patent Application No. 2021902857, filed Sep. 2, 2021, the content of which is hereby incorporated by reference in its entirety into this disclosure.

### FIELD OF THE INVENTION

This invention generally relates to sets of antibodies that specifically bind and allow visualization and quantification of a plurality of target antigens in a biological sample using multispectral tissue slide scanners, including methods and reagents related thereto.

### BACKGROUND

Multiplexed imaging has taken on increasing importance for practitioners in biomedical research and in clinical medicine/pathology. The ability to visualise multiple, specific molecules from an entire tissue sample in a single image provides a powerful tool for both research and clinical medicine applications. For example, this ability allows the spatial arrangement of different cell types to be determined, having applications for both health and disease management and treatment.

In medicine, detection of target molecules, particularly proteins, that serve as biomolecular markers or “biomarkers” within tissue samples is desirable in helping to identify the type of therapy that a patient is likely to respond to, i.e., for theragnostic applications. In one example patients with a cancer arising in a particular tissue may be grouped for different therapy according to the biomarkers that are detected within that tissue. In pathology it may also be necessary to quantify the number of cells expressing a particular target protein before a recommendation for a particular therapy can be made.

In this context, the presence, abundance, and localisation of molecules associated with immune checkpoint pathways is proving of particular use in clinical medicine and is the subject of intensive ongoing clinical research. For example, the expression of the molecule PD-L1 within tissue from patients with certain cancers has proven to be a predictor of the efficacy of certain immunotherapeutic drugs (i.e., anti-PD-1/PD-L1 immune checkpoint inhibitors).

In many countries the level of PD-L1 expression in a patient's tissue is used as a theragnostic test to determine a patient's eligibility for such drugs. Given the very large number of immune checkpoint inhibitors currently in clinical trial it is highly likely that the number of such predictive and/or theragnostic tests used in clinical research and clinical medicine will grow rapidly in coming years.

Unfortunately, current clinical practice in theragnostic tests is based on immunohistochemical technology that is decades old and semiquantitative at best, while development of more accurate quantitative tests suffers from a lack of useful protocols that will enable widespread exploitation of new technology, such as multiplexed tissue imaging. In particular, there is a lack of adequate laboratory tests that can be applied to human tissue routinely obtained in a clinical setting (formalin-fixed paraffin-embedded “FFPE” tissue) and can be used to accurately quantify immune cells and molecules involved in immune checkpoint pathways in different microenvironments across entire tissue sections. The lack of such accurate quantitative tests impedes the determination of biomarkers that correlate with clinical responses to new immune therapies in clinical trials, and subsequently reduces the ability to select patients best suited for particular immunotherapies, with consequent costs in ineffective treatments and unnecessary side effects.

In one current example, the immune checkpoint molecule PD-L1 is currently detected using a single colour immunohistochemistry (IHC) assay only, before selecting patients for anti-PD-1/PD-L1 therapy. This single colour assay allows for the qualitative detection of the PD-L1 protein only. Detection is carried out in formalin-fixed, paraffin-embedded (FFPE) tissues from many different cancer types (e.g., melanoma, non-small cell lung cancer, breast cancer, gastric cancer, and kidney cancer, but not limited thereto).

Due to the qualitative nature of this method, expert anatomical pathologists find it difficult to determine accurately and consistently parameters such as the percentage of cancer cells and immune cells expressing PD-L1, and the intensity of the staining (which is commonly reduced to a digital readout (+ vs −) without assessing expression in the greater context of the tumour microenvironment) (Lu et al. 2019; Humphries et al. 2019).

Accordingly, there is a need in the art for new and improved methods of detecting the presence and/or abundance of various targets of immune checkpoint inhibitors in various cells and tissues as new immune checkpoint inhibitors reach clinical trials and clinical practice. There is also a need in the art for new and improved methods of detecting the presence and/or abundance of PD-L1 and/or PD-1 in various cells or tissues, including cancer cells and in cancerous tissues.

It is an object of the invention to go at least some way towards addressing these needs by providing at least one antibody panel and a method of using such for the multiplex immunofluorescence detection of the presence and/or abundance of at least one immune checkpoint molecule, including PD-L1, PD-1 and at least three or four further target antigens from a single planar tissue sample and/or that will at least provide the public with a useful choice.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### SUMMARY OF THE INVENTION

In one aspect the invention relates to an antibody panel comprising at least four different targeting antibody-fluorophore pairs, wherein at least one targeting antibody-fluorophore pair is an antibody-fluorophore conjugate (Ab-FP conjugate), and wherein at least two of the targeting antibody-fluorophore pairs comprise antibodies of the same species and/or isotype.

In another aspect the present invention relates to an antibody panel comprising at least four different targeting antibody-fluorophore pairs, wherein each targeting antibody-fluorophore pair binds a different target antigen selected from: a T-cell related marker antigen selected from the group including CD3, CD4, CD8, foxp3, T-bet, GATA-3, Granzyme B, Perforin and TIA-1, an immune checkpoint molecule antigen selected from the group including PD-1, PD-L1, PD-L2, CTLA-4, TIGIT, TIM-3, LAG-3, VISTA, CD112, CD155, Ceacam-1, Galectin-3, LSECtin, CVRL4 and PVRL4, a tumour cell marker antigen selected from the group including Sox10, S100, PRAME, Pan-CK, ER, PR, HER2 and CK8, a myeloid cell marker antigen selected from the group including CD1c, CD14, CD68, CD163, CD169 and CLEC9A, and a stromal marker antigen selected from the group including CD31, CD34, CD90, LYVE-1, a-SMA and collagen.

In another aspect the invention relates to a method of determining the presence and/or abundance of a plurality of target antigens in biological sample including detecting in a planar sample of the biological sample, at least a first target antigen labelled with a first targeting antibody-fluorophore pair consisting of an Ab-FP conjugate, and at least a second target antigen labelled with a second targeting antibody-fluorophore pair, generating a single multispectral fluorescence image of the labelled planar sample using a multispectral scanner, wherein the image comprises at least four colours, wherein each colour is associated with the specific binding of a targeting antibody to a different target antigen, and determining from the image the presence and/or abundance of a plurality of target antigens, wherein i) and ii) comprise antibodies of the same species and/or isotype.

In another aspect the invention relates to a method of determining the presence and/or abundance of a plurality of target antigens in biological sample including detecting at least four target antigens in a planar sample of the biological sample, wherein each target antigen is labelled by a different targeting antibody fluorophore pair, generating a multispectral fluorescence image of the planar sample using a multispectral scanner, wherein the image including at least four colours, wherein each colour is associated with the specific binding a different targeting antibody-fluorophore pair to a different target antigen, and determining from the image the presence and abundance of the plurality of target antigens, wherein the plurality of target antigens is selected from the including: T-cell related marker antigens selected from the group consisting of CD3, CD4, CD8, foxp3, T-bet, GATA-3, Granzyme B, Perforin and TIA-1, immune checkpoint molecule antigens selected from the group including PD-1, PD-L1, PD-L2, CTLA-4, TIGIT, TIM-3, LAG-3, VISTA, CD112, CD155, Ceacam-1, Galectin-3, LSECtin, CVRL4 and PVRL4, tumour cell marker antigens selected from the group including Sox10, S100, PRAME, Pan-CK, ER, PR, HER2 and CK8, myeloid cell marker antigens selected from the group consisting of CD1c, CD14, CD68, CD163, CD169 and CLEC9A, and stromal marker antigens selected from the group including CD31, CD34, CD90, LYVE-1, a-SMA and collagen.

In another aspect the invention relates to a method of identifying the presence and/or abundance of a plurality of cell types in a biological sample including: labelling at least four target antigens in a planar sample of the biological sample with at least four different targeting antibody-fluorophore pairs, using a multispectral scanner to generate a multispectral image of the labelled planar sample by detecting the fluorescence emission spectra of each fluorophore from each different targeting antibody-fluorophore pair, and determining the presence and/or abundance of a plurality of different cell types in the planar sample based on the fluorescence emission spectra detected, optionally with reference to a suitable reference control, wherein at least one of the targeting antibody fluorophore pairs is an antibody-fluorophore conjugate (Ab-FP conjugate), and wherein at least two of the targeting antibody-fluorophore pairs comprise antibodies of the same species and/or isotype.

In another aspect the invention relates to a method of identifying the presence and/or abundance of a plurality of cell types in a biological sample including: labelling at least four target antigens in a planar sample of the biological sample with at least four different targeting antibody-fluorophore pairs, using a multispectral scanner to generate a single multispectral image of the labelled planar sample by detecting the fluorescence emission spectra of each fluorophore from each different targeting antibody-fluorophore pair, and determining the presence and/or abundance of a plurality of different cell types in the planar sample based on the fluorescence emission spectra detected, optionally with reference to a suitable reference control, wherein each targeting antibody-fluorophore pair specifically binds a different target antigen selected from: a T-cell related marker antigen selected from the group including CD3, CD4, CD8, foxp3, T-bet, GATA-3, Granzyme B, Perforin and TIA-1, an immune checkpoint molecule antigen selected from the group including PD-1, PD-L1, PD-L2, CTLA-4, TIGIT, TIM-3, LAG-3, VISTA, CD112, CD155, Ceacam-1, Galectin-3, LSECtin, CVRL4 and PVRL4, a tumour cell marker antigen selected from the group including Sox10, S100, PRAME, Pan-CK, ER, PR, HER2 and CK8, a myeloid cell marker antigen selected from the group including CD1c, CD14, CD68, CD163, CD169 and CLEC9A, and a stromal marker antigen selected from the group including CD31, CD34, CD90, LYVE-1, a-SMA and collagen.

In another aspect the invention relates to a method of making an antibody panel for an immune checkpoint disease or condition including: identifying an indicative set of four biomarkers for the immune checkpoint disease or condition, obtaining a candidate targeting antibody fluorophore pair for each biomarker in the indicative set, labelling in a planar biological sample of the immune checkpoint disease or condition, the at least four biomarkers in i) using the candidate targeting antibody-fluorophore pairs in ii), using a multispectral scanner to generate a single multispectral image comprising the fluorescence emission spectra of each fluorophore in each targeting antibody fluorophore pair in ii) identifying in the multispectral image the presence and/or abundance of each labelled biomarker, wherein each labelled biomarker is identified in the image as a different colour associated with the fluorescence emission spectra of each fluorophore in each targeting antibody-fluorophore pair, and selecting the candidate targeting antibody fluorophore pairs that can be identified in the image in iv) as an antibody panel for the immune checkpoint disease or condition, wherein at least one of the candidate targeting antibody fluorophore pairs is an Ab-FP conjugate, and wherein at least two of the targeting antibody-fluorophore pairs in ii) comprise antibodies of the same species and/or isotype.

In another aspect the invention relates to a method of making an antibody panel for an immune checkpoint disease or condition including: identifying an indicative set of four biomarkers for the immune checkpoint disease or condition, obtaining a candidate targeting antibody fluorophore pair for each biomarker in the indicative set, labelling in a planar biological sample of the immune checkpoint disease or condition, the at least four biomarkers in i) using the candidate targeting antibody-fluorophore pairs in ii), using a multispectral scanner to generate a single multispectral image comprising the fluorescence emission spectra of each fluorophore in each targeting antibody fluorophore pair in ii) identifying in the multispectral image the presence and/or abundance of each labelled biomarker, wherein each labelled biomarker is identified in the image as a different colour associated with the fluorescence emission spectra of each fluorophore in each targeting antibody-fluorophore pair, and selecting the candidate targeting antibody fluorophore pairs that can be identified in the image in iv) as an antibody panel for the immune checkpoint disease or condition, wherein at least one biomarker in i) includes a T-cell related marker antigen selected from the group including CD3, CD4, CD8, foxp3, T-bet, GATA-3, Granzyme B, Perforin and TIA-1, and at least one biomarker in i) includes an immune checkpoint molecule antigen selected from the group including PD-1, PD-L1, PD-L2, CTLA-4, TIGIT, TIM-3, LAG-3, VISTA, CD112, CD155, Ceacam-1, Galectin-3, LSECtin, CVRL4 and PVRL4.

In another aspect the invention relates to a method of making an iterated antibody panel including: establishing a first antibody panel that detects a core set of target antigens, the first antibody panel comprising a core set of targeting antibody-fluorophore pairs including at least one antibody-fluorophore conjugate (Ab-FP conjugate), identifying a second set of core target antigens establishing a second antibody panel that detects the second set of core antigens in ii) by replacing at least one of the targeting antibody-fluorophore pairs in i) that is not an Ab-FP conjugate with a substitute targeting antibody-fluorophore pair that specifically binds to at least one target antigen in ii), obtaining a single multispectral image of the fluorescence emission spectra of each fluorophore from the targeting antibody fluorophore pairs in iii) by detecting in a planar biological sample the core set of target antigens from ii), identifying in the multispectral image the second set of core target antigens from ii), wherein each core target antigen in ii) is identified in the image as a different colour that is associated with the specific binding of a different targeting antibody-fluorophore pair to a target antigen, and selecting an iterated antibody panel comprising at least one substituted targeting antibody fluorophore pair that can be identified in the image in iv) as an iterated antibody panel, wherein at least one of the target antigens in ii) has been specifically labelled by at least one Ab-FP conjugate, and at least one different target antigen in ii) has been specifically labelled by at least one substitute targeting antibody-fluorophore pair from iii).

Various embodiments of the different aspects of the invention as discussed above are also set out below in the detailed description of the invention, but the invention is not limited thereto.

Other aspects of the invention may become apparent from the following description which is given by way of example only and with reference to the accompanying drawings.

### BRIEF DESCRIPTION OF THE DRAWINGS

The invention will now be described with reference to the figures in the accompanying drawings.

FIG. 1—Unmixed image (greyscale) of antibody-fluorophore+DAPI labelled melanoma-infiltrated lymph node tissue showing PD-L1 expression

Unmixed image of one of seven colours (DL755)+DAPI detected simultaneously from an antibody-fluorophore labelled melanoma-infiltrated lymph node tissue section showing the distribution of PD-L1+ tumour and immune cells only. PD-L1+ cells labelled with anti-PD-L1 primary Ab, coupled with DL755 conjugated secondary Ab, are shown as bright and/or grey.

FIG. 2—Unmixed image (greyscale) of an antibody-fluorophore+DAPI labelled melanoma-infiltrated lymph node tissue showing PD-1 expression

Unmixed image of one of seven colours (AF488)+DAPI detected simultaneously from an antibody-fluorophore labelled melanoma-infiltrated lymph node tissue section showing the distribution of PD-1+ immune cells only. PD-1+ cells labelled with anti-PD-1 primary Ab, coupled with AF488 conjugated secondary Ab, are shown as bright and/or grey.

FIG. 3—Unmixed image (greyscale) of an antibody-fluorophore+DAPI labelled melanoma-infiltrated lymph node tissue showing Sox10 expression

Unmixed image of one of seven colours (AF546)+DAPI detected simultaneously from an antibody-fluorophore labelled melanoma-infiltrated lymph node tissue section showing the distribution of Sox10+ tumour cells only. Sox10+ cells labelled with anti-Sox10 primary Ab, coupled with AF546 conjugated secondary Ab, are shown as bright and/or grey.

FIG. 4—Unmixed image (greyscale) of an antibody-fluorophore+DAPI labelled melanoma-infiltrated lymph node tissue showing CD68 expression

Unmixed image of one of seven colours (BV480)+DAPI detected simultaneously from an antibody-fluorophore labelled melanoma-infiltrated lymph node tissue section showing the distribution of CD68+ macrophages only. CD68+ cells labelled with anti-CD68 primary Ab, coupled with BV480 conjugated secondary Ab, are shown as bright and/or grey.

FIG. 5—Unmixed image (greyscale) of an antibody-fluorophore+DAPI labelled melanoma-infiltrated lymph node tissue showing Foxp3 expression

Unmixed image of one of seven colours (DL680)+DAPI detected simultaneously from an antibody-fluorophore labelled melanoma-infiltrated lymph node tissue section showing the distribution of Foxp3+ regulatory T cells only. Foxp3+ cells labelled with anti-Foxp3 primary Ab, coupled with DL680 conjugated secondary Ab, are shown as bright and/or grey.

FIG. 6—Unmixed image (greyscale) of an antibody-fluorophore+DAPI labelled melanoma-infiltrated lymph node tissue showing CD8 expression

Unmixed image of one of seven colours (AF594)+DAPI detected simultaneously from an antibody-fluorophore labelled melanoma-infiltrated lymph node tissue section showing the distribution of CD8+ cells only. CD8+ cells labelled with an anti-CD8-AF594 antibody conjugate are shown as bright and/or grey.

FIG. 7—Unmixed image (greyscale) of an antibody-fluorophore+DAPI labelled melanoma-infiltrated lymph node tissue showing the nuclear stain DAPI only

Unmixed image of one of seven colours (DAPI) detected from an antibody-fluorophore labelled melanoma-infiltrated lymph node tissue section showing the nuclear stain DAPI only.

FIG. 8—Combined unmixed image (greyscale) of an antibody-fluorophore+DAPI labelled melanoma-infiltrated lymph node tissue

The combination of the unmixed images in FIGS. 1 to 7 showing all seven colours (six fluorophores+DAPI) detected simultaneously from an antibody-fluorophore labelled melanoma-infiltrated lymph node tissue section.

FIG. 9—Unmixed image (greyscale) of an antibody-fluorophore+DAPI labelled melanoma-infiltrated lymph node tissue showing PD-L1 expression

Unmixed image of one of six colours (AF647)+DAPI detected simultaneously from an antibody-fluorophore labelled melanoma-infiltrated lymph node tissue section showing the distribution of PD-L1+ tumour and immune cells only. PD-L1+ cells labelled with anti-PD-L1 primary Ab, coupled with AF647 conjugated secondary Ab, are shown as bright and/or grey.

FIG. 10—Unmixed image (greyscale) of an antibody-fluorophore+DAPI labelled melanoma-infiltrated lymph node tissue showing PD-1 expression

Unmixed image of one of six colours (AF546)+DAPI detected simultaneously from an antibody-fluorophore labelled melanoma-infiltrated lymph node tissue section showing the distribution of PD-1+ immune cells only. PD-1+ cells labelled with anti-PD-1 primary Ab, coupled with AF546 conjugated secondary Ab, are shown as bright and/or grey.

FIG. 11—Unmixed image (greyscale) of an antibody-fluorophore+DAPI labelled melanoma-infiltrated lymph node tissue showing Sox10 expression

Unmixed image of one of six colours (AF488)+DAPI detected simultaneously from an antibody-fluorophore labelled melanoma-infiltrated lymph node tissue section showing the distribution of Sox10+ tumour cells only. Sox10+ cells labelled with anti-Sox10 primary Ab, coupled with AF488 conjugated secondary Ab, are shown as bright and/or grey.

FIG. 12—Unmixed image (greyscale) of an antibody-fluorophore+DAPI labelled melanoma-infiltrated lymph node tissue showing CD68 expression

Unmixed image of one of six colours (BV480)+DAPI detected simultaneously from an antibody-fluorophore labelled melanoma-infiltrated lymph node tissue section showing the distribution of CD68+ macrophages only. CD68+ cells labelled with anti-CD68 primary Ab, coupled with BV480 conjugated secondary Ab, are shown as bright and/or grey.

FIG. 13—Unmixed image (greyscale) of an antibody-fluorophore+DAPI labelled melanoma-infiltrated lymph node tissue showing CD8 expression

Unmixed image of one of six colours (AF594)+DAPI detected simultaneously from an antibody-fluorophore labelled melanoma-infiltrated lymph node tissue section showing the distribution of CD8+ cells only. CD8+ cells labelled with an anti-CD8-AF594 antibody conjugate are shown as bright and/or grey.

FIG. 14—Unmixed image (greyscale) of an antibody-fluorophore+DAPI labelled melanoma-infiltrated lymph node tissue showing the nuclear stain DAPI only

Unmixed image of one of six colours (DAPI) detected from an antibody-fluorophore labelled melanoma-infiltrated lymph node tissue section showing the nuclear stain DAPI only.

FIG. 15—Combined unmixed image (greyscale) of an antibody-fluorophore+DAPI labelled melanoma-infiltrated lymph node tissue

The combination of the unmixed images in FIGS. 9 to 14 showing all six colours (five fluorophores+DAPI) detected simultaneously from an antibody-fluorophore labelled melanoma-infiltrated lymph node tissue section.

FIG. 16—Unmixed image (greyscale) of Ab-FP+DAPI labelled melanoma-infiltrated lymph node tissue showing PD-L1 expression

Unmixed image of one of seven colours (6 Ab-FP+DAPI) detected simultaneously from an Ab-FP labelled melanoma-infiltrated lymph node tissue section showing the distribution of PD-L1+ tumour and immune cells only. PD-L1+ cells labelled with anti-PD-L1 primary Ab, coupled with AF594 conjugate secondary Ab, are shown as bright and/or grey.

FIG. 17—Unmixed image (greyscale) of Ab-FP+DAPI labelled melanoma-infiltrated lymph node tissue showing PD1 expression

Unmixed image of one of seven colours (6 Ab-FP+DAPI) detected simultaneously from an Ab-FP labelled melanoma-infiltrated lymph node tissue section showing the distribution of PD-1+ immune cells only. PD-1+ cells labelled with anti-PD-1-AF555 antibody conjugate are shown as bright and/or grey.

FIG. 18—Unmixed image (greyscale) of Ab-FP+DAPI labelled melanoma-infiltrated lymph node tissue showing Sox10 expression

Unmixed image of one of seven colours (6 Ab-FP+DAPI) detected simultaneously from an Ab-FP labelled melanoma-infiltrated lymph node tissue section showing the distribution of Sox10+ tumour cells only. Sox10+ cells labelled with anti-Sox10 primary Ab, coupled with AF488 conjugated secondary Ab, are shown as bright and/or grey.

FIG. 19—Unmixed image (greyscale) of Ab-FP+DAPI labelled melanoma-infiltrated lymph node tissue showing CD68 expression

Unmixed image of one of seven colours (6 Ab-FP+DAPI) detected simultaneously from an Ab-FP labelled melanoma-infiltrated lymph node tissue section showing the distribution of CD68+ macrophages only. CD68+ cells labelled with anti-CD68 primary Ab, coupled with BV480 conjugated secondary Ab, are shown as bright and/or grey.

FIG. 20—Unmixed image (greyscale) of Ab-FP+DAPI labelled melanoma-infiltrated lymph node tissue showing Foxp3 expression

Unmixed image of one of seven colours (6 Ab-FP+DAPI) detected simultaneously from an Ab-FP labelled melanoma-infiltrated lymph node tissue section showing the distribution of Foxp3+ regulatory T cells only. Foxp3+ cells labelled with anti-Foxp3 primary Ab, coupled with DL755 conjugated secondary Ab, are shown as bright and/or grey

FIG. 21—Unmixed image (greyscale) of Ab-FP+DAPI labelled melanoma-infiltrated lymph node tissue showing CD8 expression

Unmixed image of one of seven colours (6 Ab-FP+DAPI) detected simultaneously from an Ab-FP labelled melanoma-infiltrated lymph node tissue section showing the distribution of CD8+ cells only. CD8+ cells labelled with an anti-CD8-AF647 antibody conjugate are shown as bright and/or grey.

FIG. 22—Unmixed image (greyscale) of Ab-FP+DAPI labelled melanoma-infiltrated lymph node tissue showing the nuclear stain DAPI only

Unmixed image of one of seven colours (6 Ab-FP+DAPI) detected simultaneously from an Ab-FP labelled melanoma-infiltrated lymph node tissue section showing the nuclear stain DAPI only.

FIG. 23—Combined unmixed image (greyscale) of Ab-FP+DAPI labelled melanoma-infiltrated lymph node tissue

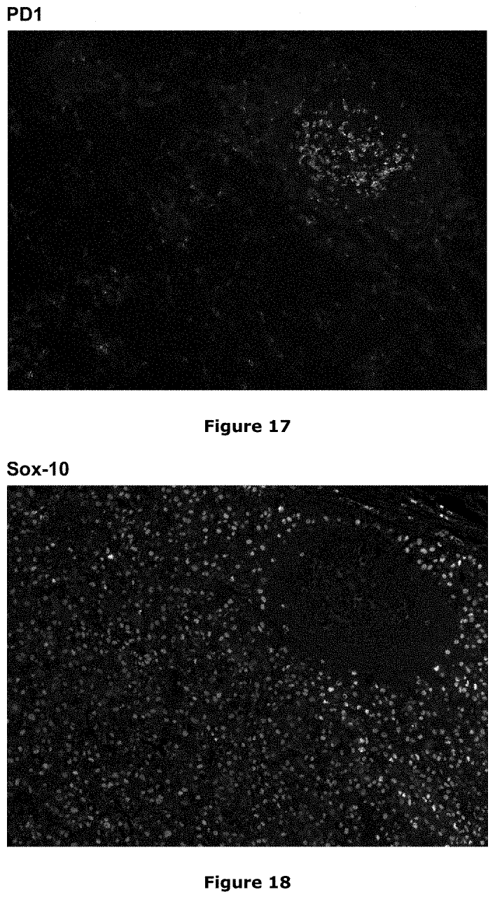

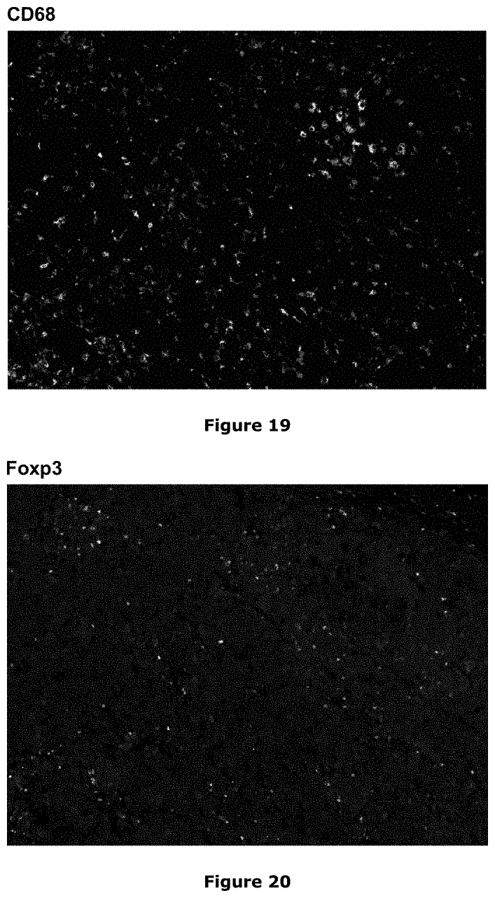

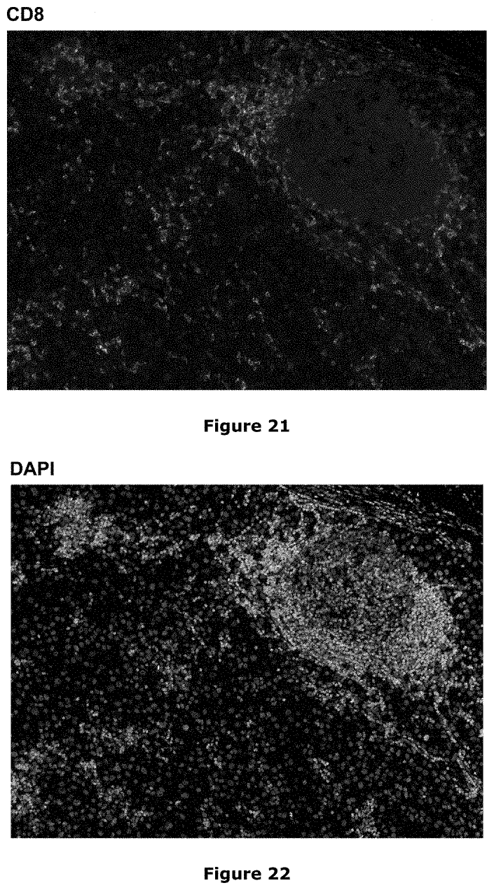

The combination of the unmixed images in FIGS. 16 to 22 showing all seven colours (6 Ab-FP+DAPI) detected simultaneously from an Ab-FP labelled melanoma-infiltrated lymph node tissue section.

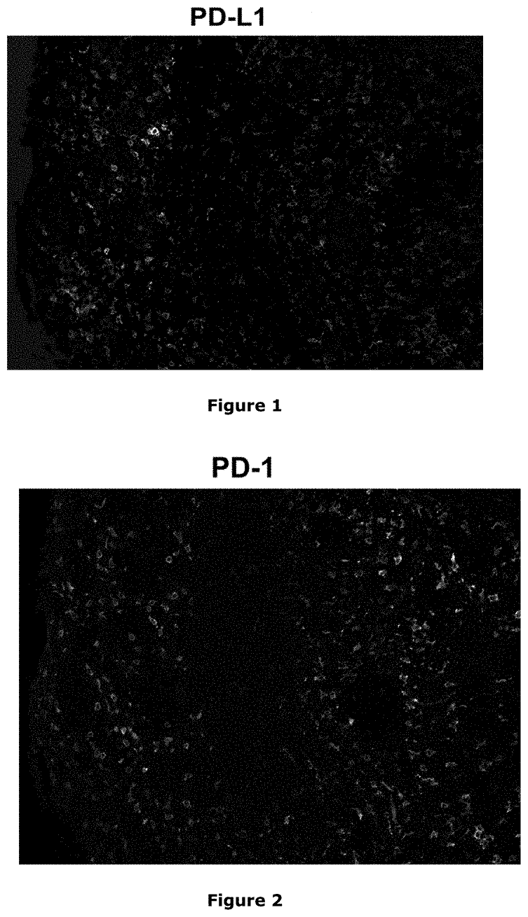

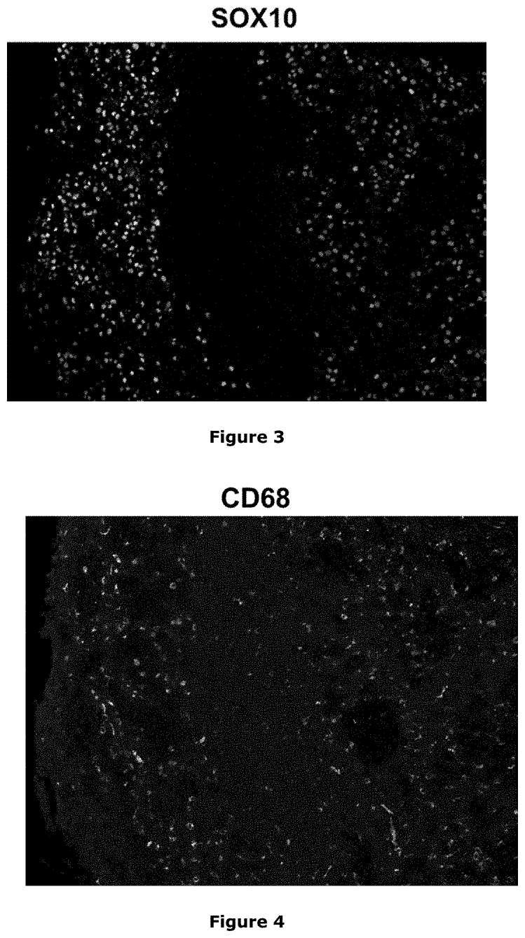

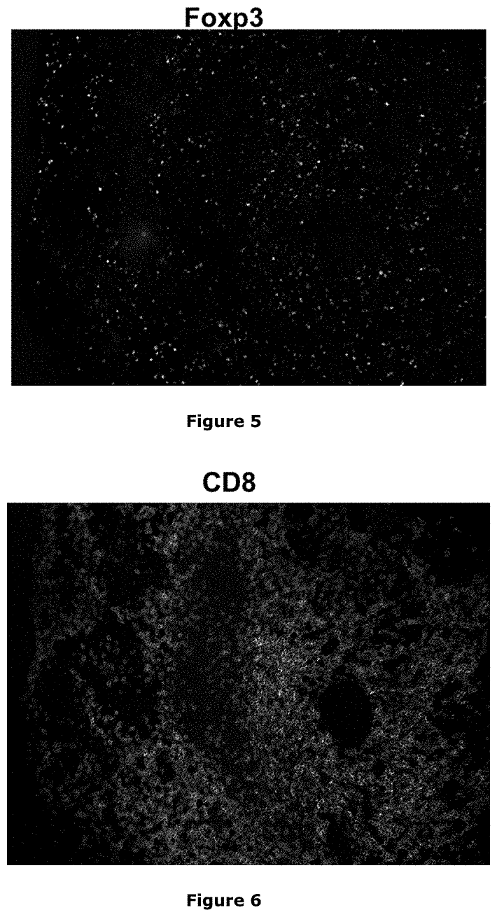

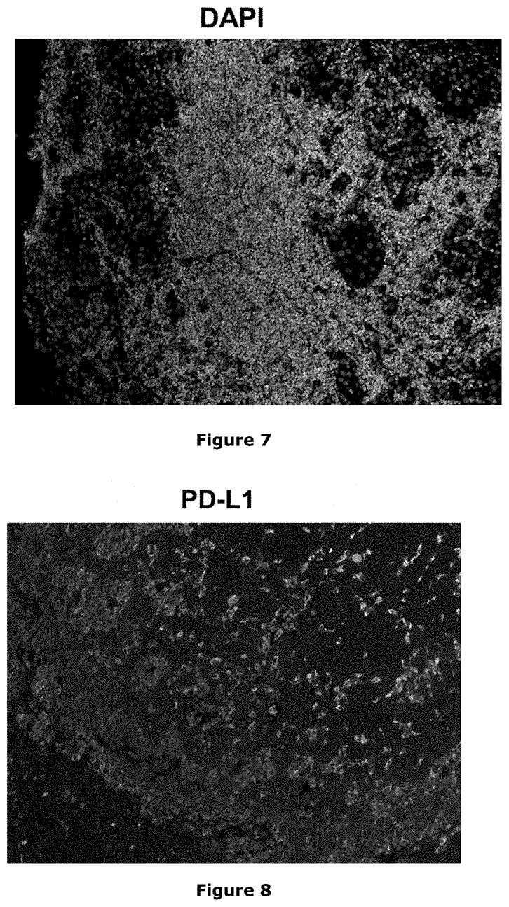

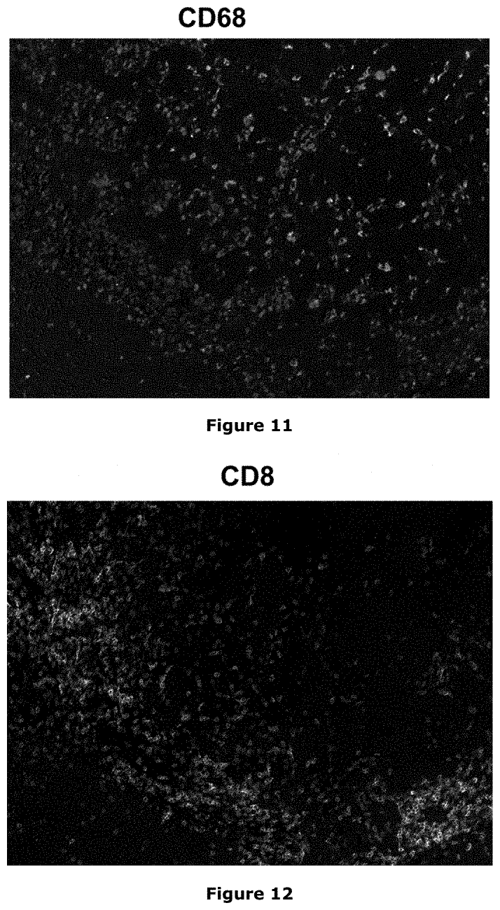

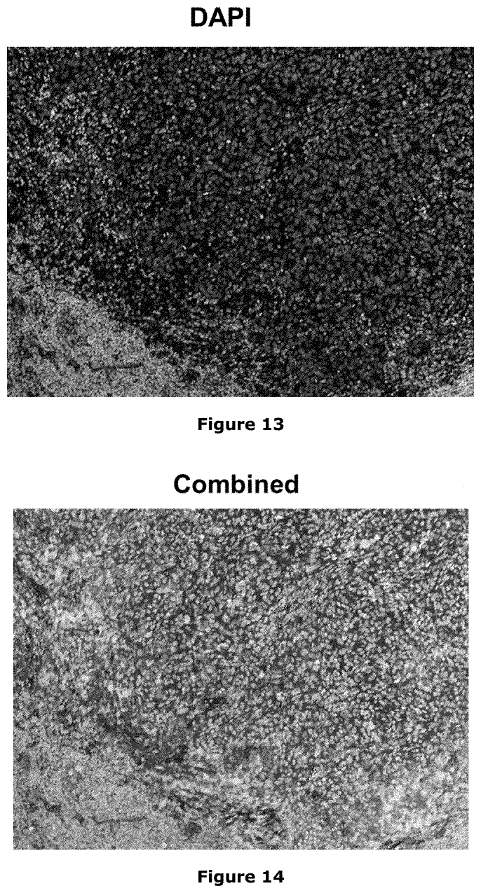

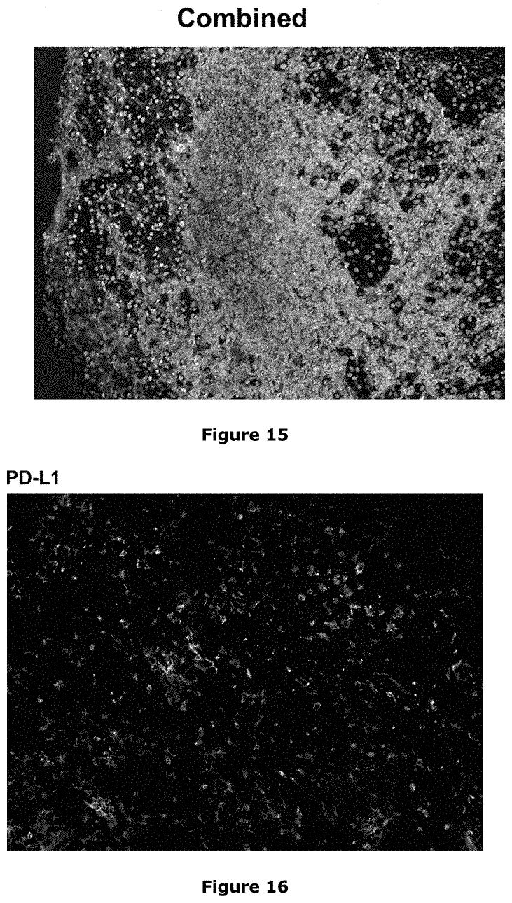

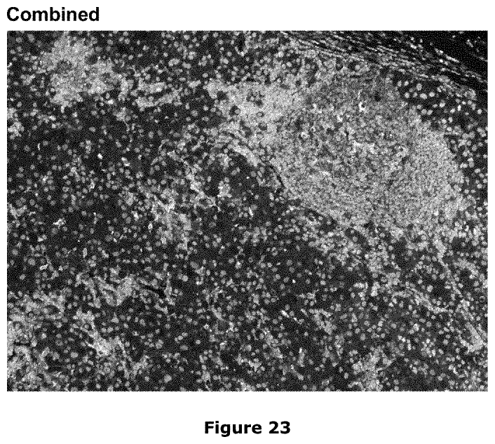

All images in FIGS. 1 to 23 were acquired multispectrally using the Vectra Polaris.

### DETAILED DESCRIPTION OF THE INVENTION

### Definitions

Unless otherwise specified, all technical and scientific terms used herein are to be understood as having the same meanings as is understood by one of ordinary skill in the relevant art to which this disclosure pertains. It is also believed that practice of the present invention can be performed using standard immunology, histology, cell biology, molecular biology, pharmacology and biochemistry protocols and procedures as known in the art.

The singular form “a”, “an” and “the” include plural referents unless the context clearly dictates otherwise. These articles refer to one or to more than one (i.e., to at least one). The term “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”.

The term “about” as used in connection with a numerical value throughout the specification and the claims denotes an interval of accuracy, familiar and acceptable to a person skilled in the art. In general, such interval of accuracy is +/−10%.

Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

The term “exemplary” means serving as a non-limiting example, instance, or illustration. As utilized herein, the terms “e.g.,” and “for example” set off lists of one or more non-limiting aspects, examples, instances, or illustrations.

The following definitions are presented to better define the present invention and as a guide for those of ordinary skill in the art in the practice of the present invention.

All patents and publications, including all sequences disclosed within such patents and publications, referred to herein are expressly incorporated by reference.

The term “targeting antibody fluorophore pair” and grammatical variations thereof as used herein means a matched set of an antibody and a fluorophore in which the antibody and the fluorophore are used together to label a target antigen in a multiplex immunofluorescence labelling protocol. The antibody in a targeting antibody fluorophore pair is a primary antibody that binds specifically and directly to a target antigen. In some embodiments the target antigen is naturally occurring on a biomolecule. In one embodiment the biomolecule is a biomarker.

In some embodiments a targeting antibody fluorophore pair is an antibody fluorophore conjugate (Ab-FP) comprising a single Ab portion having one or more covalently bound FPs. In some embodiments, an Ab-FP comprises a plurality of FPs covalently bound to a single Ab. Antibody fluorophore conjugates as contemplated herein are described in PCT/IB2021/051581, which is herein incorporated by reference in its entirety.

In some embodiments a targeting antibody fluorophore pair is a combination of a primary antibody that specifically binds a target antigen and a secondary labelling antibody-fluorophore conjugate that specifically binds the primary antibody. In this embodiment the targeting antibody fluorophore pair is used in a “sandwich” assay; i.e., an indirect immunofluorescence labelling protocol using primary targeting antibodies and secondary labelling antibodies as known in the art.

The term “unique” including grammatical variations and abbreviations thereof when used in reference to an antibody or a fluorophore in a targeting antibody fluorophore pair means that in any composition containing multiple targeting antibody fluorophore pairs, the antibodies and the fluorophores comprised in each pair are different from any other antibodies or fluorophores comprised in any other pair comprised in, or that may be used with, the composition. A composition comprising at least two unique pairs means that the antibodies and the fluorophores in each of the two pairs are different from each other. Likewise, a composition comprising at least three unique pairs means that the antibodies and FPs in each of the three pairs are different from each other.

The terminology “maximum fluorescence excitation and emission wavelength (Ex/Em) and grammatical variations thereof as used herein means the wavelength (nm) of maximum excitation (Ex) of a fluorophore and the wavelength of maximum emission (Em) of that fluorophore.

The terminology “fluorescence excitation and emission spectra” and grammatical variations thereof as used herein refers to a plurality of excitation and emission wavelengths of a given fluorophore that are distinctive for that given fluorophore and that are detected using a multispectral scanner as described herein.

A “multispectral scanner” (also called a “multispectral slide scanner” herein) and grammatical variations thereof as used herein refers to a device that is able to collect data over a variety of different wavelength ranges including multispectral slide scanners, tissue imaging systems and automated qualitative pathology imaging systems. Microscopes and flow cytometers are not multispectral scanners as used herein. Specifically contemplated as an embodiment of any of the aspects of the invention that employ multispectral scanning or a multispectral scanner, the multispectral scanner is a Vectra Polaris.

The terminology “labelled”, and “labelling” and grammatical variations thereof as used herein with reference to a targeting antibody fluorophore pair refers to a matched set of an antibody and a fluorophore that are used together with at least one other matched set of an antibody and a fluorophore in a multispectral imaging method as described herein. In some embodiments labelling is carried out using an Ab-FP conjugate as described herein. In some embodiments labelling is carried out using an indirect method, including a sandwich assay, as described herein.

The term “antibody” and grammatical variations thereof refers to an immunoglobulin molecule having a specific structure that interacts (binds) specifically with a molecule comprising its cognate antigen. In some embodiments the antigen is the antigen that was used for synthesizing the antibody. This antigen is known as the target antigen.

The phrase “each Ab is different from each other Ab” means that each Ab specifically binds a different target antigen.

As used herein, the term “antibody” and grammatical variations thereof broadly refers to full length antibodies and may also include certain antigen binding portions and/or fragments thereof. Also included are monoclonal and polyclonal antibodies, multivalent and monovalent antibodies, multi-specific antibodies (for example bi-specific antibodies), chimeric antibodies, human antibodies, humanized antibodies, and antibodies that have been affinity matured and antigen binding portions and/or fragments thereof.

Specifically contemplated herein an “antibody portion” of a targeting antibody broadly refers to full length antibodies and may also include certain antigen binding portions and/or fragments thereof. Also included are monoclonal and polyclonal antibodies, multivalent and monovalent antibodies, multi-specific antibodies (for example bi-specific antibodies), chimeric antibodies, human antibodies, humanized antibodies, and antibodies that have been affinity matured and antigen binding portions and/or fragments thereof.

A “targeting antibody” is an antibody or antigen binding portion thereof that specifically binds a target antigen.

A “target antigen” that is “specifically bound” or “specifically labelled” (including grammatical variations thereof) by targeting antibody is an antigen that binds preferentially to the target antibody e.g., has less than 25%, or less than 10%, or less than 1% or less than 0.1% cross-reactivity with a non-target antibody. In some embodiments, the target antigen is a protein antigen.

As used herein a “target antigen” means an antigen on a biomolecule in a sample that is directly bound by, and thereby specifically labelled by an antibody pair described herein. Target antigens as used herein are not primary antibodies to which secondary antibodies can be bound. However, primary antibodies that specifically bind target antigens can, in some embodiments, be considered ligands that are labelled by secondary antibodies to provide an antibody pair according to the present invention and as described herein.

Usually, a targeting antibody will have a binding affinity (dissociation constant (Kd) value), for the antigen or epitope of no more than 10-6, or 10-7M, preferably less than about 10-8M, more preferably less than about 10-9M, or 10-10, or 10-11 or 10-12M. Binding affinity may be assessed using surface plasma resonance [see, for example U.S. Pat. No. 7,531,639 or 6,818,392, each of which is incorporated herein by reference].

The term “cell-type” and grammatical variations thereof as used herein means a group of cells that are defined by the shared presence of one or more expressed target antigens. As used herein a “cell-type” can be any size grouping of cells that express the one or more target antigens, such as a population of cells, a sub-population of cells or a smaller group. By way of non-limiting example, a cell type may be a population of immune cells as known in the art, such as T cells, B-cells, or a sub-population of such cells such as invariant natural killer T cells (iNKT) cells.

The terms “planar sample” and “planar biological sample” and grammatical variations thereof as used herein refer to a substantially planar, i.e., two-dimensional samples of biological material containing cells or any combination of biomolecule complexes, cellular organelles, sub-cellular structures, or cellular debris (aka “cellular material”). Planar samples may be obtained by sectioning a three-dimensional sample containing cells or cellular material into sections and mounting the sections onto a planar surface. Planar samples may also be obtained by growing or depositing cells or cellular material on a planar surface, or by adsorbing or absorbing cells or cellular material to a planar surface. In a specific embodiment of the invention, a planar biological sample is a tissue section.

The terminology a “colour” that is “associated with the specific binding of antibody in a targeting antibody fluorophore pair” and grammatical variations thereof is a colour represented in a fluorescence image that directly correlates with the fluorescence emission spectra of an FP in a targeting antibody fluorophore pair when targeting antibody is specifically bound to a target antigen in situ in a planar biological sample.

The terminology “a suitable control image” and grammatical variations thereof is well understood by a skilled worker and means an image that has been generated for use as an acceptable comparative control as would be recognized by a person of skill in the art. In one non-limiting example, a suitable control image may be an image of an unlabelled tissue section. In another example, a suitable control image may be an image of a tissue section taken from an earlier time point where progression of a disease or condition is being monitored, or from a healthy individual, or from an individual before disease onset, or after medical treatment, but not limited thereto. It is believed that the generation of a suitable control image may be carried out by a skilled worker with reference to the relevant art in combination with the methods and reagents provided by the present disclosure.

The term “biomarker” and grammatical variations thereof is used herein as understood by the skilled person and encompasses a biomolecule that comprises a target antigen, as well as a cell or cellular structure or cellular sub-structure that comprises the biomolecule. In some embodiments the biomolecule is a protein, a carbohydrate, a lipid, or a combination thereof; e.g., a glycolipid or glycoprotein, but not limited thereto. In one embodiment the biomolecule is a protein. In one embodiment the biomolecule is a protein that is expressed on or in a cell.

When used descriptively a “biomarker” means a biomolecule that is known to be associated with and/or indicative of a particular biological process, feature, object, state, status, or function. In one non-limiting example a biomarker is indicative of a cell type, a cellular function, or a cellular process. In some embodiments, the biomarker is a functional marker, for example a marker of a cellular process that occurs in a number of different cells. In such a case, the relative expression of a biomarker may allow discrimination of different cell types, cellular structures and/or cellular sub-structures by enabling sufficient labelling of the biomarker using a targeting antibody-fluorophore pair as described herein to determine the presence and/or abundance of the biomarker. In one example a biomarker is associated with a disease state or the status of disease progression, but not limited thereto. In one embodiment the biomarker is a theragnostic biomarker, or one of a set of theragnostic biomarkers, for a proposed course of therapy.

As used herein the term “immune checkpoint molecule” and grammatical variations thereof refers to both inhibitory and stimulatory immune checkpoint molecules that exert inhibitory or stimulatory effects on immune responses. In some embodiments the immune checkpoint molecule is a tumour associated or tumour cell associated immune checkpoint molecule, and in others is associated with non-malignant immune cells or stromal cells, for example T cells, macrophages and other myeloid cells, or fibroblastic cells.

In one example an immune checkpoint molecule is PD-1 (also known as CD279—UniProt Acc #Q15116) or PD-L1 (also known as CD274—UniProt Acc #Q9NZQ7).

As used herein the phrase “immune checkpoint related disease or condition” and grammatical variations thereof refers to a disease where immune checkpoint molecules are targeted with therapeutic effect, including but not limited to cancer, infectious disease, autoimmune disease, and organ transplantation.

As used herein the phrase “known to be associated with” in reference to a biomarker and grammatical variations thereof means that the biomarker is indicative of a biological feature, molecule, structure, state, or status of an organism from which it is measured. In some examples, the presence or absence of a biomarker may be indicative of any one or all of a biological feature, molecule, structure, state, or status. In other examples, the absolute or relative abundance of a biomarker may be indicative of any one or all of a biological feature, molecule, structure, state, or status.

As used herein, the term “abundance” encompasses “relative abundance”.

The term “relative abundance” as used herein refers to both a) the number or % of labelled cells of a given cell type or having a given target antigen or biomarker as compared to the total cells of that cell type or having a given target antigen or biomarker and b) the number or % of labelled cells of a given cell type or having a given target antigen or biomarker as compared to the total cells of a different cell type, including a different cell type having the same or a different given target antigen or biomarker. The skilled worker will appreciate that the same meaning a set out above applies to this term when used herein to describe the relative abundance of a target antigen and/or labelled target antigen.

The terminology “determining the abundance of a cell type” and grammatical variations thereof means determining the absolute number of cells that comprise a particular biomarker of interest according to the methods as described herein. In some embodiments determining the abundance means identifying the number of cells in a multispectral immunofluorescence image of a sample that have a fluorescence emission intensity that is greater than the predetermined background level of autofluorescence of the sample.

The terminology “determining the relative abundance of a cell type”” and grammatical variations thereof means determining the % number of cells that comprise a particular biomarker of interest from among the total population of that cell type present in a planar sample including determining relative abundances of different cell types by comparing the relative abundances of particular cell types to each other, again according to the methods as described herein.

The terminology “multispectral imaging of an entire tissue section” and grammatical variations thereof means that the entire section of the tissue sample that is present on a slide or other carrier supporting the section for labelling and imaging is scanned using a multispectral scanner. In some embodiments the image created from that scan encompasses all of, or part of, the tissue section present on the slide or carrier.

The term “patient” as used herein is used interchangeably with “subject” and means the same thing. A patient or subject is an animal. Preferably the animal is a mammal. Preferably the mammal includes human and non-human mammals such as cats, dogs, horses, pigs, cows, sheep, deer, mice, rats, primates (including gorillas, rhesus monkeys and chimpanzees), possums and other domestic farm or zoo animals, but not limited thereto. Preferably, the mammal is human. The term “pre-determined” and grammatical variations thereof when used herein to describe a cell type, disease or condition means that the cell type, disease, or condition is known and may be selected by a skilled worker in view of the present disclosure and the art.

The term “clinically relevant levels” and grammatical variations thereof as used herein means that the presence and/or abundance of a biomarker detected according to a method as described herein is recognized as clinically actionable by a skilled worker.

As used herein the terminology “in high abundance” and grammatical variations thereof used to describe a biomarker, cell, cell type, cellular structure or sub-structure means that the abundance or relative abundance of the biomarker, cell, cell type, cellular structure or sub-structure in a sample is recognized by a skilled worker as clinically actionable.

The term “clinically actionable” and grammatical variations thereof as used herein refers to the presence and/or abundance of at least one biomarker in a multispectral immunofluorescence image produced according to a method as described herein, wherein the presence and/or abundance of the detected biomarker provides to the clinician clear and compelling evidence that therapeutic intervention is required.

As used herein the term “unique colour” and grammatical variations thereof means a colour specifically corresponding to the spectra of fluorescence energy emitted from a particular FP in a targeting antibody fluorophore pair as described herein.

The term “comprising” as used in this specification and claims means “consisting at least in part of”; that is to say when interpreting statements in this specification and claims which include “comprising”, the features prefaced by this term in each statement all need to be present but other features can also be present. Related terms such as “comprise” and “comprised” are to be interpreted in similar manner.

The term “consisting essentially of” as used herein means the specified materials or steps and those that do not materially affect the basic and novel characteristic(s) of the claimed invention.

The term “consisting of” as used herein means the specified materials or steps of the claimed invention, excluding any element, step, or ingredient not specified in the claim.

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

### DETAILED DESCRIPTION

The inventors have unexpectedly found that certain combinations of targeting antibody fluorophore pairs as described herein can be used in multiplex immunofluorescence staining protocols designed for multispectral scanners to detect sets of biomarkers that are useful in clinical cancer immunology research and/or in theragnostic methods for various cancer therapies and other fields of medicine where immune checkpoints are involved in disease processes or targeted with therapy. Moreover, the inventors have identified that rapid iteration of an antibody panel around a core set of targeting antibody-fluorophore pairs (comprising primary “targeting” antibodies and secondary “detection” antibodies) as described herein allows for previously unachievable rapid development of specific methods of multiplex immunofluorescence detection for multispectral scanners, and/or theragnosis of various immunologically important molecules related to a number of diseases and conditions, including cancers.

The ability to simultaneously and rapidly detect sets of biomarkers of interest according to the methods described herein provides the clinicians and clinical researchers with distinct, unanticipated and unexpected advantages in terms of disease diagnosis, patient prognosis, theragnostic applications, and for other clinical applications requiring for example, discrimination between multiple cell types in a tissue based on the presence and/or abundance and/or relative abundance of a particular biomarker, and those cells' expression of particular molecules, including therapeutic targets and the molecules they bind.

One component of the inventive technology described herein relates to the inventor's determination that a set of target antigens can be detected across an entire planar biological sample with a multispectral scanner using a panel of targeting antibody-fluorophore pairs comprising at least one directly labelled antibody fluorophore pair (an Ab-FP conjugate), wherein the species and/or isotype of an antibody in at least two targeting antibody-fluorophore pairs in the panel are the same.

Multispectral slide scanners have been designed to overcome some of the limitations of multiplex immunofluorescence staining when using conventional epifluorescence or confocal microscopes. First the ability to scan entire planar samples, for example sections of human tissue samples, enables capture of data from across the entire sample; in contrast previous microscopy approaches typically only enabled small proportions of the sample to be captured as images. Second multispectral detection methods enabled development of methods to distinguish between a wider range of fluorophores than had previously been possible using bandpass filters on conventional microscopes. However staining protocols available for multispectral slide scanners have been highly iterative and inconvenient, e.g., the Opal staining system for Polaris, which uses sequential labelling of up to 8 antibodies with enzymatic reactions that deposit different fluorophores on the tissue. One of the reasons for the use of these iterative stains such as the Opal method is that they were needed to deliver bright enough signal for detection by these multispectral slide scanning instruments such as the Vectra Polaris from Akoya Biosciences.

The inventors have found that the Vectra Polaris instrument is useful for detecting the binding of target antigens by directly labelled antibodies comprising covalently bound fluorophores (Ab-FP conjugates), or indirectly labelled by binding of a primary or “targeting antibody” which is subsequently labelled by antibody-antibody binding using a secondary antibody-fluorophore conjugate. A skilled worker appreciates that while the latter detection method may have been used in some contexts in conventional microscopy, it has not been reported nor promoted for use in multispectral scanners, particularly the Vectra Polaris instrument. In addition, the inventors have determined that combining both directly-conjugated primary antibodies and indirect detection of primary antibodies with secondary antibody fluorophore conjugates had major advantages for use on multispectral scanners, including the Vectra Polaris instrument.

These advantages are particularly evident in the ability to combine primary antibodies of the same species and/or isotype in a single antibody panel used in a multiplex immunofluorescence tissue staining protocol that detects fluorescence labelled antibodies with a multispectral scanner. The inventors believe that they are the first to report such use with multispectral scanning instruments.

A skilled person at the time this invention was made considered, in relation to multispectral scanning instruments, that these instruments would not be sensitive enough to detect such combinations of antibodies as described above. In contrast, the inventors have identified that by selecting certain combinations of fluorophores for secondary antibody labelling, they are able to provide for both detection and de-convolution of the fluorescence emission signals when using such secondary labels in 6-plex panels of antibodies. The inventors have further identified that only a subset of those fluorophores is suitable for conjugating to primary antibodies.

The importance of the ability to use directly-conjugated antibodies within panels of antibodies designed for use on multispectral slide scanners can be appreciated by those skilled in the art of multiplex immunofluorescence detection in conventional epifluorescence or confocal microscopes. Most previous protocols used on these microscopes have employed indirect labelling protocols or “sandwich assays” as they are known in the art. These protocols provide strong signal and can employ a wide range of primary and secondary reagents that are commercially available, as well as proprietary unlabelled primary antibodies.

In these protocols, a primary antibody that specifically binds a target antigen is subsequently labelled by a secondary antibody-fluorophore conjugate. Binding of the secondary antibody to the primary antibody is dictated by secondary antibody species and isotype, i.e., the secondary antibody specifically binds a portion of the primary antibody, this portion varying between antibodies based on the species of animal in which the antibody was produced, and the isotype of the particular antibody produced against an antigen.

As the skilled reader appreciates, this means that once a primary antibody of a given species and/or isotype is included into multi-antibody “panel” for multiplexed detection of several antigens, introduction in the panel of a further primary antibody of the same species and/or isotype is precluded. Hence an acknowledged technical limitation of multiplex immunofluorescence detection methods lies in the limited number of different species and isotypes of antibodies that can be used in indirect labelling protocols, especially when using monoclonal antibodies where antigen specificity has been validated.

The skilled reader appreciates that multiplex immunofluorescence staining protocols currently employed in conventional epifluorescence or confocal microscopes require the use of different secondary antibodies for each fluorophore label to be applied in a multiplex staining protocol. These secondary antibodies, comprising a conjugated fluorophore, label targets by reacting with different species and/or isotypes of primary or “targeting” antibodies. In some examples, especially when the skilled user wishes to use only monoclonal antibodies to ensure specificity for the antigenic targets, the species of a primary or “targeting” antibody is a mouse, rat, rabbit, or hamster monoclonal antibody. Likewise, the isotype of the primary antibody within the species type can vary, including for example, IgG1, IgG2a, IgG2b or IgG3, but not limited thereto. The skilled person will appreciate that based on the disclosure herein, other species of monoclonal antibodies can be used in the panels and methods described herein.

Because secondary labeling antibodies will cross react based on species and/or isotype similarity when used for multiplex staining, there is a requirement to find different antibody species and/or isotypes for each target in a multiplex indirect immunostaining protocol; e.g., a 6-plex indirect immunostaining protocol. This requirement becomes especially limiting in FFPE sections where many primary or “targeting” antibodies don't work. A skilled worker appreciates that due to the lack of available antibodies, effective staining for a particular antigen in FFPE may be limited to the use of a single primary or “targeting” antibody. This limitation immediately rules out using another antibody of the same species/isotype in a multiplex immunofluorescence using indirect labelling.

The disadvantage of this limitation becomes immediately apparent when developing a new panel of monoclonal antibodies and/or when iterating a panel of monoclonal antibodies to add, delete or substitute antibody fluorophore pairs.

As the number of suitable primary or targeting antibodies within a panel is identified, the species and/or isotypes of the antibodies that will effectively bind their required target antigens is fixed. Each required primary or “targeting’ antibody therefore excludes the use of that same species and/or isotype of antibody elsewhere in the multiplex stain.

In the context of multispectral slide scanning, the inventors have overcome the technical limitations above by designing antibody panels with sets of targeting antibody-fluorophore pairs, where at least one of the targeting antibody-fluorophore pairs in the set is an Ab-FP conjugate. Because the antibody comprised in an Ab-FP conjugate is directly conjugated to the fluorophore, labelling of the target antigen does not require a secondary antibody. Additionally, the binding of the Ab-FP conjugate to the target antigen is target antigen specific. The antibody portion of an Ab-FP conjugate as described herein does not bind to any target ligand in the sample other than the target antigen. The antibody portion of the Ab-FP conjugates does not bind to any secondary antibodies as described herein.

In a particularly advantageous embodiment described herein are sets of target antigens that can be detected in a single multiplex immunofluorescence image obtained by a multispectral slide scan of a planar biological sample where the sample has been immunolabelled with a combination of 4-12 targeting (primary) antibodies where two or more of these antibodies can be of the same species or isotype. Provided that only one of these targeting antibodies is detected with a detection (secondary) antibody conjugated to a fluorophore, all the other targeting antibodies used may be of the same species or isotype because they are directly conjugated to different fluorophores (Ab-FPs).

Moreover, by maintaining invariant at least one Ab-FP conjugate within an antibody panel as described herein, significant tolerance for variation (relative to an antibody panel comprising all indirectly labelled antibodies) in the selection of other targeting antibody-fluorophore pairs in the antibody panel can introduced. In some embodiments at least two, three, four, five or six Ab-FP conjugates are maintained invariant. This flexibility allows iteration of antibody panels of choice with surprising rapidity as compared to methods of generating antibody panels used for indirect immunostaining as known in the art.

This ability to rapidly iterate antibody panels around a core set of targeting antibody-fluorophore pairs as described herein is unknown in the art and provides specific technical advantages that could not have been foreseen by the skilled worker in this field.

Biomarkers

A person skilled in the art recognizes that a biomolecular marker or “biomarker” is a term of art describing a biomolecule, in this case a protein, the presence of which is considered to be theragnostic, diagnostic or prognostic for a particular biological event or context, such as a disease, or a cell population. Detection of one or more “biomarkers” by various means can be used for a large number of research and clinical purposes.

For example, in the present disclosure, a biomarker may be any target biomolecule, preferably a protein, wherein the target biomolecule is detected and visualized according to the methods described herein by specific binding of a labelled targeting antibody described herein to a target antigen comprised on the biomolecule. The target antigen is present on the biomolecule which itself will be present in or on a cell. A biomarker may be used to detect a cell, cell type, cellular structure, or sub-structure, but not limited thereto. In one embodiment, the biomolecule is a protein or antigen comprising portion thereof.

In one exemplary situation, a target antigen on an CD21 protein is labelled using a targeting antibody fluorophore pair as described herein, wherein the target antigen is present on a protein, wherein that protein is present on a cell and serves as one of several biomarkers of mature-B cells and follicular dendritic cells (but not limited thereto).

Multispectral immunofluorescence detection of a plurality of biomarkers for a number of different theragnostic, diagnostic and prognostic purposes is specifically contemplated as part of the methods described herein. The inventors believe that in view of the present disclosure, a skilled worker using the methods of the invention as described herein can simultaneously detect the presence and/or abundance and/or relative abundance of a plurality of cells or cell types comprising a core set of target antigens as described herein as follows.

The skilled worker can select an appropriate core set of at least four, five, preferably at least six targeting antibodies that specifically bind to at least four, five, preferably at least six target antigens, each comprised on a pre-determined biomarker respectively. The selected antibodies are then labelled with a fluorophore as described herein to form a targeting antibody fluorophore pair as described herein.

In this manner the methods described herein are employed by the skilled person in the theragnosis, diagnosis and/or prognosis of many different diseases or conditions. In some embodiments the diseases or conditions are immunological diseases or conditions. In one embodiment the diseases are cancers. In one embodiment the cancer is selected from the group consisting of melanoma, cervical carcinoma, breast carcinoma, ovarian carcinoma, hepatocellular carcinoma, esophageal squamous cell carcinoma, gastric/gastroesophageal junction adenocarcinoma, endometrial adenocarcinoma, head and neck squamous cell carcinoma, non-small cell lung cancer and urothelial carcinoma.

The methods described herein can also be employed clinically to identify patient populations and sub-populations by immuno-populations and sub-populations of cells, and to predict and monitor the effects of drugs and/or candidate drugs on target antigen expression, but not limited thereto.

The inventors believe that the advantages of the methods and reagents provided herein, particularly for theragnostic applications, are numerous when viewed in comparison to methods available in the art.

The only currently FDA-approved companion diagnostic (for anti-PD-1/PD-L1 immune checkpoint inhibitor therapy) is the PD-L1 immunohistochemistry (IHC) test. A number of PD-L1 IHC tests are approved with different antibody clones, staining protocols, scoring system, and the localization of PD-L1 detection. However, IHC tests are unable to label more than one marker (i.e., PD-L1) per tissue section and cannot precisely distinguish between PD-L1 expressed in tumour cells and PD-L1 in immune cells.

A couple of pre-designed multiplexed PD-1/PD-L1 IHC panels are available for research purpose (Akoya Biosciences, MOTIF PD-L1 auto melanoma kit & lung cancer kit), but the staining process using these panels is labour-intensive, takes several days to complete, and introduces the risk of multiple rounds of human error.

In contrast, the methods described herein are distinctly advantageous for the clinician in enabling equivalent staining protocols to be carried out in FFPE tissue sections as little as two hours and iterated within a matter of days. These advantages are facilitated by the speed of the multispectral scan employed in the methods as described herein. In some embodiments generating a multispectral fluorescence image of a sample is done in less than 4 h, 3 h, 2 h, preferably less than 1 h.

Antibody Fluorophore Labelling of Formalin Fixed Paraffin Embedded (FFPE) Tissue

Antibody fluorophore labelling of FFPE tissue using traditional multiplexed IF methods (i.e., indirect detection) is mostly limited to two to four colour detection (a 5th colour is reserved for nuclear stains, e.g., DAPI), due to the 1) limited availability of primary antibodies in different isotypes (if from the same host animal) and host species, 2) limited availability of fluorophores that can be well separated by conventional IF microscope filters.

Regarding the first limitation, it is well known to those skilled in the art that when using indirect IF, there are limited options for detection of targeting (primary) antibodies applied simultaneously to tissue. Specifically, each targeting (primary) antibody needs to be detected by a detection antibody (a secondary antibody-fluorophore conjugate) that cannot bind the other targeting antibodies. In practice this means that in multiplexed panels designed for FFPE tissue, each targeting antibody must come either from a different species of animal (typically mouse, rat rabbit) or be a different isotype of IgG from the same species that can be distinguished by commercially-available detection antibodies (e.g., mouse IgG1, IgG2a, IgG2b, IgG3).

For monoclonal targeting antibodies, where specificity is restricted to a single epitope and therefore able to be fully characterised, this means effectively multiplexed antibodies are typically composed of one antibody from each of the 7 following classes: rabbit monoclonal IgG; rat monoclonal IgG; hamster monoclonal IgG; mouse monoclonal IgG1; mouse monoclonal IgG2a; mouse monoclonal IgG2b; mouse monoclonal IgG3. In conventional practice this has meant that building multiplexed panels becomes increasingly difficult with indirect IF as the number of colours increases: as each new targeting antibody is selected it needs to be of a different class from all of those already in the panel.

In practice when using commercially-available targeting antibodies, many antigens have validated monoclonal antibodies of more than one class; but very few have 3 or more options (see the Abminer database of monoclonal antibodies https://discover.nci.nih.gov/abminer/ or Human Protein Atlas https://www.proteinatlas.org/). Hence many 6-colour panels that a person skilled in the art may want to build from monoclonal antibodies are simply not possible with indirect IF.

The Opal IHC technique for use on multispectral scanning instruments such as the Polaris gets around this problem by using sequential staining with individual targeting antibodies and enzyme-linked secondary antibodies that then activate tyramide chemistry to deposit fluorescent stains of different emissions spectra (“colours”) on the tissue.

Currently available Opal IHC protocols allow multiplexed IF staining of FFPE tissue using up to seven to nine colours. However, manual Opal staining takes a minimum of three to four days to complete the entire staining cycle. Furthermore, developing and optimising a seven colour Opal staining panel can take six to eight weeks (please see this webpage for more information: https://www.akoyabio.com/product-support/opal-multiplex-immunohistochemistry#Opal-FAQ), and users require considerable knowledge and experience of IHC techniques to design and develop Opal staining panels. Additionally, the Opal platform is not compatible with frozen tissue sections.

Thus, the Opal multiplex IHC technique is not yet widely used in clinical diagnostics and research labs.

In providing the methods and reagents as described herein the inventors have overcome a number of the disadvantages set forth above by combining direct and indirect labelling of target antigens in methods of labelling and detecting a core set of target antigens that are theragnostic, diagnostic and/or prognostic for immune checkpoint related diseases and conditions, including cancer.

The methods described herein may comprise sandwich assays and may encompass primary labelling of a target antigen with a primary or “targeting” antibody that specifically binds a target antigen, followed by detection of the primary or “targeting” antibody by secondary labelling with an antibody-fluorophore conjugate, the fluorophore then being detected by fluorescence emission. Additionally, or in the alternative, the methods described may require the Ab-FPs as described herein, wherein the antibody portion of the Ab-FP is a primary antibody that specifically binds the target antigen, and the FP portion is covalently bound to the primary Ab. A skilled worker in the art appreciates that following the methods described herein leverage the advantages of direct detection of a target antigen by an Ab-FP as described herein, as well as the advantages provided in certain circumstances by secondary labelling as employed in sandwich assays to provide a unique and unexpended solution to the problem of accurately predicting the efficacy of drug therapy, particularly cancer therapy.

One of the distinct advantages provided by the present invention is the use of antibodies directly conjugated to fluorophores (i.e., directly conjugated antibodies) to rapidly allow the multispectral detection and imaging of an entire slide/section. In some embodiments the advantages also encompass the use of a plurality of directly conjugated antibodies to rapidly and simultaneously allow the multispectral detection and imaging of an entire slide/section. In one non-limiting example, this is done using the Vectra Polaris.

The inventors believe that they are the first to provide a skilled worker with the ability to rapidly generate new tests for clinical research or theragnostic tests that visualise a set of clinically relevant molecular markers in a single image using a multispectral slide scanner, enabling quantification of relative expression of molecules of interest in different cells. For example, following the methods described herein a multiplex fluorescence image of a planar biological sample comprising at least four and up to twelve target antigens can be generated in less than fifteen to thirty minutes using only Ab-FPs. In other embodiments where a combination of Ab-FPs and sequential use of targeting and detection antibodies is employed, immune-staining can be completed in under 6 h, 5 h, 4 h, 3 h, 2 h, preferably under 1 hour. In some embodiments the ability to perform out such theragnostic tests within hours as provided herein offers clear and distinct advantages over existing technologies and offers the skilled worker the means to improve the efficiency and accuracy of theragnostic pathology in many fields.

The inventors also believe they are the first to provide a skilled worker with the ability to combine targeting antibodies of the same species or isotype in the same panel for use on a multispectral slide scanner without needing to resort to Opal staining or other methods that require each individual antibody to be applied separately. The unanticipated ability to use Ab-FPs within these panels enables antibodies of the same species or isotype to readily be used within the same panel, either as Ab-FPs that comprise different fluorophores, or as combinations where one of the targeting antibodies is detected with a fluorophore-conjugated detection antibody and the other targeting antibodies of the same species or isotype are all Ab-FPs with other fluorophores.

The inventors also believe they are the first to provide a skilled worker with a range of fluorophore combinations that can be included in antibody panels capable of distinguishing between 6 different antigens alongside a DNA stain using a multispectral slide scanner.

The inventors further believe that the skilled person readily appreciates that the methods and reagents described herein provide a non-obvious technical solution that enables the development of new applications in biomedical research as described herein. The methods and reagents described herein further enable the development of rapid theragnostic methods that can be employed to accelerate the selection of optimal therapeutic regimen for patients. For example, in some embodiments the methods disclosed herein can be employed by the skilled person for molecular and immune profiling and disease management of cancer, including but not limited to lung, breast, colorectal, hepatocellular, endometrial, ovarian and melanoma cancers by choice of the appropriate plurality of biomarkers (for example with reference to (Hofman, 2019) (Sood, 2016) and (Majtahed, 2011), the disclosures of which are all expressly incorporated herein by reference in their entireties).

Accordingly, in one aspect the invention relates to an antibody panel comprising at least four different targeting antibody-fluorophore pairs,

wherein at least one targeting antibody-fluorophore pair is an antibody-fluorophore conjugate (Ab-FP conjugate), and

wherein at least two of the targeting antibody-fluorophore pairs comprise antibodies of the same species and/or isotype.

In one embodiment of this and all other aspects of the invention set forth herein, all antibodies comprised in the antibody panel are monoclonal antibodies.

In one embodiment of this and all other aspects of the invention, the targeting antibody fluorophore pairs consist of monoclonal antibodies and fluorophores.

In one embodiment the antibody panel comprises at least five different targeting antibody fluorophore pairs. In one embodiment the antibody panel comprises at least six different targeting antibody fluorophore pairs.

In one embodiment the antibody panel comprises at least seven, eight, nine, ten, preferably eleven different targeting antibody fluorophore pairs.

In one embodiment each targeting antibody fluorophore pair binds a different target antigen selected from the groups consisting of

a T-cell marker antigen,

an immune checkpoint molecule antigen,

a tumour cell marker antigen,

a myeloid cell marker antigen, and

a stromal marker antigen.

In one embodiment at least one of the target antigens is a).

In one embodiment at least one of the target antigens is b).

In one embodiment at least one of the target antigens is a) and at least one of the target antigens is b).

In one embodiment at least one of the target antigens is a), at least one of the target antigens is b) and at least one of the target antigens is c), d) or e) or a combination thereof.

In one embodiment at least one of the target antigens is a), at least one of the target antigens is b) and at least one of the target antigens is c) or d) or a combination thereof.

In one embodiment at least one of the target antigens a), at least one of the target antigens b) and at least one of the target antigens c) or e) or a combination thereof.

In one embodiment at least one of the target antigens a), at least one of the target antigens b) and at least one of the target antigens d) or e) or a combination thereof.

In one embodiment at least one of the target antigens a), at least one of the target antigens is b) and at least one of each of the target antigens is c), d), and e).

In one embodiment a) is selected from the group consisting of CD3, CD4, CD8, foxp3, T-bet, GATA-3, Granzyme B, Perforin and TIA-1 or a combination thereof. In one embodiment a) is selected from the group consisting of CD3, CD8, foxp3, and TIA-1 or a combination thereof. In one embodiment a) is CD8 or foxp3. In one embodiment a) is CD8. In one embodiment is foxp3.

In one embodiment b) is selected from the group consisting of PD-1, PD-L1, PD-L2, CTLA-4, TIGIT, TIM-3, LAG-3, VISTA, CD112, CD155, Ceacam-1, Galectin-3, LSECtin, CVRL4 and PVRL4 or a combination thereof. In one embodiment b) is selected from the group consisting of PD-1, PD-L1, PD-L2, TIGIT and TIM-3 or a combination thereof. In one embodiment b) is PD-1 or PD-L1 or a combination thereof. In one embodiment b) is PD-1. In one embodiment b) is PD-L1.

In one embodiment c) is selected from the group consisting of Sox10, S100, PRAME, Pan-CK, ER, PR, HER2 and CK8 or a combination thereof. In one embodiment c) is selected from the group consisting of Sox10, PRAME, Pan-CK, and CK8 or a combination thereof. In one embodiment c) is Sox10.

In one embodiment d) is selected from the group consisting of CD1c, CD14, CD68, CD163, CD169 and CLEC9A or a combination thereof. In one embodiment d) is CD68 or CD163 or a combination thereof. In one embodiment d) is CD163. In one embodiment d) is CD68.

In one embodiment e) is selected from the group consisting of CD31, CD34, CD90, LYVE-1, alpha-Smooth Muscle Actin (a-SMA) and collagen or a combination thereof. In one embodiment e) is LYVE-1 or a-SMA or a combination thereof. In one embodiment e) is LYVE-1. In one embodiment e) is a-SMA.

In one embodiment a) is CD8, b) comprises PD-1 and PD-L1, c) is Sox10 and d) is CD68.

In one embodiment a) comprises CD8 and foxp3, b) comprises PD-1 and PD-L1 antibodies, c) is Sox10 and d) is CD68.

In one embodiment each different targeting antibody fluorophore pair comprises an antibody selected from the group consisting of

an anti-T cell marker antibody,

an anti-immune checkpoint molecule antibody,

an anti-tumour cell marker antibody,

an anti-myeloid cell marker antibody, and

an anti-stromal marker antibody.

In one embodiment at least one of the targeting antibody fluorophore pairs comprises a). In one embodiment at least one of the targeting antibody fluorophore pairs comprises b).

In one embodiment at least one of the targeting antibody fluorophore pairs comprises a) and at least one of the targeting antibody fluorophore pairs comprises b).

In one embodiment at least one of the targeting antibody fluorophore pairs comprises a), at least one of the targeting antibody fluorophore pairs comprises b) and at least one of the targeting antibody fluorophore pairs comprises c), d) or e) or a combination thereof.

In one embodiment at least one of the targeting antibody fluorophore pairs comprises a), at least one of the targeting antibody fluorophore pairs comprises b) and at least one of the targeting antibody fluorophore pairs comprises c) or d) or a combination thereof.

In one embodiment at least one of the targeting antibody fluorophore pairs comprises a), at least one of the targeting antibody fluorophore pairs comprises b) and at least one of the targeting antibody fluorophore pairs comprises c) or e) or a combination thereof.

In one embodiment at least one of the targeting antibody fluorophore pairs comprises a), at least one of the targeting antibody fluorophore pairs comprises b) and at least one of the targeting antibody fluorophore pairs comprises d) or e) or a combination thereof.

In one embodiment at least one of the targeting antibody fluorophore pairs comprises a), at least one of the targeting antibody fluorophore pairs comprises b) and at least one of each of the targeting antibody fluorophore pairs comprises c), d), and e).

In one embodiment a) is selected from the group consisting of anti-CD3, CD4, CD8, foxp3, T-bet, GATA-3, Granzyme B, Perforin and anti-TIA-1 antibodies or a combination thereof. In one embodiment a) is selected from the group consisting of anti-CD3, CD8, foxp3, and anti-TIA-1 antibodies or a combination thereof. In one embodiment a) is an anti-CD8 or anti-foxp3 antibody or combination thereof.

In one embodiment b) is selected from the group consisting of anti-PD-1, PD-L1, PD-L2, CTLA-4, TIGIT, TIM-3, LAG-3, VISTA, CD112, CD155, Ceacam-1, Galectin-3, LSECtin, CVRL4 and anti-PVRL4 antibodies or a combination thereof. In one embodiment b) is selected from the group consisting of anti-PD-1, PD-L1, PD-L2, TIGIT and anti-TIM-3 antibodies or a combination thereof. In one embodiment b) is an anti-PD-1 or anti-PD-L1 antibody or a combination thereof.

In one embodiment c) is selected from the group consisting of anti-Sox10, S100, PRAME, Pan-CK, ER, PR, HER2 and anti-CK8 antibodies or a combination thereof. In one embodiment c) is selected from the group consisting of anti-Sox10, PRAME, Pan-CK, and anti-CK8 antibodies or a combination thereof. In one embodiment c) is an anti-Sox10 antibody.

In one embodiment d) is selected from the group consisting of anti-CD1c, CD14, CD68, CD163, CD169 and anti-CLEC9A antibodies or a combination thereof. In one embodiment d) is an anti-CD68 or anti-CD163 antibody or a combination thereof. In one embodiment d) is an anti-CD68 antibody.

In one embodiment e) is selected from the group consisting of anti-CD31, CD34, CD90, LYVE-1, alpha-Smooth Muscle Actin (a-SMA) and anti-collagen antibodies or a combination thereof. In one embodiment e) is an anti-LYVE-1, anti-CD31 or anti-a-SMA antibody or a combination thereof.

In one embodiment a) is an anti-CD8 antibody, b) comprises anti-PD-1 and anti-PD-L1 antibodies, c) is anti-Sox10 antibody and d) is an anti-CD68 antibody.

In one embodiment a) comprises an anti-CD8 antibody and an anti-foxp3 antibody, b) comprises anti-PD-1 and anti-PD-L1 antibodies, c) is anti-Sox10 antibody and d) is an anti-CD68 antibody.

The following fluorophores are contemplated for use in targeting antibody fluorophore pairs in all of the aspects of the invention set forth herein.

In one embodiment fluorophores used in the targeting antibody fluorophore pairs described herein have maximum excitation and emission wavelengths (Ex/Em) selected from the group consisting of 348/395 nm, 404/448 nm, 405/421 nm, 405/510 nm, 405/570 nm, 405/603 nm, 405/646 nm, 405/711 nm, 407/421 nm, 415/500 nm, 436/478 nm, 490/515 nm, 494/520 nm, 495/519 nm, 485/693 nm, 496/578, 532/554 nm, 566/610 nm, 590/620 nm, 650/660 nm, 650/668 nm, 652/704, 696/719 nm, 753/785 nm, 754/787 nm, 755/775 nm and 759/775 nm.

In one embodiment, the maximum fluorescence emission wavelength (Em) of at least one, two or three of the FPs is about 710 nm to about 850 nm. In one embodiment the Em of at least one, two or three of the FPs is about 753 nm to about 759 nm, preferably of 753 nm, 754 nm, 755 nm, or 759 nm. In one embodiment the Em of one of the FPs is 754 nm.

In one embodiment the FP is selected from the group consisting of Brilliant™ Ultraviolet 395 (BUV395) having an Ex/Em of 348/395, Brilliant™ Violet 480 (BV480) having an Ex/Em of 436/478 nm, Brilliant Violet 421™ having an Ex/Em of 405/421, Brilliant™ Violet 421 (BV421) having an Ex/Em of 407/421, Brilliant™ Violet 510 (BV510) having an Ex/Em of 405/510, Brilliant Violet 570™ having an Ex/Em of 405/570, Brilliant Violet 605™ having an Ex/Em of 405/603, Brilliant Violet 650™ having an Ex/Em of 405/646, Brilliant Violet 711™ having an Ex/Em of 405/711, BD Horizon™ V450 having an Ex/Em of 404/448, BD Horizon™ V500 having an Ex/Em of 415/500, Brilliant™ Blue 515 (BB515) having an Ex/Em of 490/515 nm, Fluorescein Isothiocyanate (FITC) having an Ex/Em of 494/520 nm, Alexa Fluor 488 (AF488) having an Ex/Em of 495/519 nm, Alexa Fluor 532 (AF532) having an Ex/Em of 532/554 nm, R-phycoerythrin (PE) having an Ex/Em of 496/578, Alexa Fluor 594 (AF594) having an Ex/Em of 590/620 nm, PE-Dazzle 594 (PE594) or PE-CF594 (CF594) having an Ex/Em of 566/610 nm, Alexa Fluor 647 (AF647) having an Ex/Em of 650/668 nm, Allophycocyanin (APC) having an Ex/Em of 650/660, DyLight 680 (DL680) having an Ex/Em of 692/712 nm, BD Horizon™ 700 (BB700) having an Ex/Em of 485/693 nm, Alexa Fluor 700 (AF700) having an Ex/Em of 696/719 nm, APC/Alexa Fluor 750 having an Ex/Em of 753/785 nm, APC/Fire 750 having an Ex/Em of 754/787 nm, APC-R700 having an Ex/Em of 652/704, APC-Cy7 having an Ex/Em of 755/775 nm, DyLight 755 (DL755) having an Ex/Em of 754/776 and AF750 having an Ex/Em of 759/775 nm.

In one embodiment the fluorophore is selected from the group consisting of BV480, AF488, AF546, AF555, DL680, AF647, AF594, and DL755.

In one embodiment the targeting antibody fluorophore pairs comprising a) comprise a fluorophore selected from the group consisting of BV480, AF488, AF546, AF555, DL680, AF647, AF594, and DL755. Preferably the fluorophore is AF488, AF594, AF647, DL680 or DL755 or a combination thereof, preferably AF488, preferably AF594, preferably AF647, preferably DL680, preferably DL755.

In one embodiment the targeting antibody fluorophore pairs comprising b) comprise a fluorophore selected from the group consisting of BV480, AF488, AF546, AF555, DL680, AF647, AF594, and DL755. Preferably the fluorophore is AF488, AF555, AF647, or DL755 or a combination thereof, preferably AF488, preferably AF555, preferably AF647, preferably DL755.

In one embodiment the targeting antibody fluorophore pairs comprising c) comprise a fluorophore selected from the group consisting of BV480, AF488, AF546, AF555, DL680, AF647, AF594, and DL755. Preferably the fluorophore is AF488, AF546, AF555 or AF594, preferably AF488, preferably AF546, preferably AF555.

In one embodiment the targeting antibody fluorophore pairs comprising d) comprise a fluorophore selected from the group consisting of BV480, AF488, AF546, AF555, DL680, AF647, AF594, and DL755. Preferably the fluorophore is BV480.

In one embodiment the targeting antibody fluorophore pairs comprising e) comprise a fluorophore selected from the group consisting of BV480, AF488, AF546, AF555, DL680, AF647, AF594, and DL755. Preferably the fluorophore is DL755.

In one embodiment at least two different targeting antibody fluorophore pairs are Ab-FP conjugates. In one embodiment at least three different targeting antibody fluorophore pairs are Ab-FP conjugates. In one embodiment at least four different targeting antibody-fluorophore pairs are Ab-FP conjugates. In one embodiment at least five different targeting antibody-fluorophore pairs are Ab-FP conjugates. In one embodiment at least six different targeting antibody-fluorophore pairs are Ab-FP conjugates.

In one embodiment at least seven, eight, nine, ten, preferably eleven different targeting antibody fluorophore pairs are Ab-FP conjugates.

In one embodiment at least one Ab-FP conjugate specifically binds a target antigen selected from the group consisting of

a T-cell marker antigen,

an immune checkpoint molecule antigen,

a tumour cell marker antigen,

a myeloid cell marker antigen, and

a stromal marker antigen.

In one embodiment at least one of the target antigens is a).

In one embodiment at least one of the target antigens is b).

In one embodiment at least one of the target antigens is a) and at least one of the target antigens is b).

In one embodiment at least one of the target antigens is a), at least one of the target antigens is b) and at least one of the target antigens is selected from one of groups c), d) and e) or a combination thereof.

In one embodiment at least one of the target antigens is a), at least one of the target antigens is b) and at least one of the target antigens is c) or d) or a combination thereof.

In one embodiment at least one of the target antigens is a), at least one of the target antigens is b) and at least one of the target antigens is c) or e) or a combination thereof.

In one embodiment at least one of the target antigens is a), at least one of the target antigens is b) and at least one of the target antigens is d) or e) or a combination thereof.

In one embodiment at least one of the target antigens is a), at least one of the target antigens is b) and at least one of each of the target antigens c), or d) and e).

In one embodiment a) is selected from the group consisting of CD3, CD4, CD8, foxp3, T-bet, GATA-3, Granzyme B, Perforin and TIA-1 or a combination thereof. In one embodiment a) is selected from the group consisting of CD3, CD8, foxp3, and TIA-1 or a combination thereof. In one embodiment a) is CD8 or foxp3. In one embodiment a) is CD8. In one embodiment a) is foxp3.

In one embodiment b) is selected from the group consisting of PD-1, PD-L1, PD-L2, CTLA-4, TIGIT, TIM-3, LAG-3, VISTA, CD112, CD155, Ceacam-1, Galectin-3, LSECtin, CVRL4 and PVRL4 or a combination thereof. In one embodiment b) is selected from the group consisting of PD-1, PD-L1, PD-L2, TIGIT and TIM-3 or a combination thereof. In one embodiment b) is PD-1 or PD-L1 or a combination thereof. In one embodiment b) is PD-1. In one embodiment b) is PD-L1.

In one embodiment c) is selected from the group consisting of Sox10, S100, PRAME, Pan-CK, ER, PR, HER2 and CK8 or a combination thereof. In one embodiment c) is selected from the group consisting of Sox10, PRAME, Pan-CK, and CK8 or a combination thereof. In one embodiment c) is Sox10.

In one embodiment d) is selected from the group consisting of CD1c, CD14, CD68, CD163, CD169 and CLEC9A or a combination thereof. In one embodiment d) is CD68 or CD163 or a combination thereof. In one embodiment d) is CD163. In one embodiment d) is CD68.

In one embodiment e) is selected from the group consisting of CD31, CD34, CD90, LYVE-1, alpha-Smooth Muscle Actin (a-SMA) and collagen or a combination thereof. In one embodiment e) is LYVE-1 or a-SMA or a combination thereof. In one embodiment e) is LYVE-1. In one embodiment e) is a-SMA.

In one embodiment a) is CD8, b) comprises PD-1 and PD-L1, c) is Sox10 and d) is CD68.

In one embodiment a) comprises CD8 and foxp3, b) comprises PD-1 and PD-L1 antibodies, c) is Sox10 and d) is CD68.

In one embodiment the Ab-FP conjugates comprising a) comprise a fluorophore selected from the group consisting of BV480, AF488, AF546, AF555, DL680, AF647, AF594, and DL755. Preferably the fluorophore is AF488, AF594, AF647, DL680 or DL755 or a combination thereof, preferably AF488, preferably AF594, preferably AF647, preferably DL680, preferably DL755.

In one embodiment the Ab-FP conjugates comprising b) comprise a fluorophore selected from the group consisting of BV480, AF488, AF546, AF555, DL680, AF647, AF594, and DL755. Preferably the fluorophore is AF488, AF555, AF647, or DL755 or a combination thereof, preferably AF488, preferably AF555, preferably AF647, preferably DL755.

In one embodiment the Ab-FP conjugates comprising c) comprise a fluorophore selected from the group consisting of BV480, AF488, AF546, AF555, DL680, AF647, AF594, and DL755. Preferably the fluorophore is AF488, AF546, AF555 or AF594, preferably AF488, preferably AF546, preferably AF555.

In one embodiment the Ab-FP conjugates comprising d) comprise a fluorophore selected from the group consisting of BV480, AF488, AF546, AF555, DL680, AF647, AF594, and DL755. Preferably the fluorophore is BV480.

In one embodiment the Ab-FP conjugates comprising e) comprise a fluorophore selected from the group consisting of BV480, AF488, AF546, AF555, DL680, AF647, AF594, and DL755. Preferably the fluorophore is DL755.

Specifically contemplated embodiments of an antibody panel comprising at least four targeting antibody fluorophore pairs as described herein are set out as i) to xviii) in Table 1. Targeting antibodies from targeting antibody fluorophore pairs are shown by their antigen target and their antibody species and isotype.

TABLE 1
Antibody panels of the invention

| | |
|---|---|
| Targeting antibody | FP |

FIGS. 1-8

| | |
|---|---|
| Ø | DAPI |
| CD68, mIgG3 | anti-mIgG3 BV480 |
| PD-1, mIgG1 | anti-mIgG1 AF488 |
| Sox10, mIgG2b | anti-mIgG2b AF546 or AF555 |
| [symbol] | Ø |
| FoxP3, rat IgG | anti-rat IgG DL680 or AF647 |
| PD-L1, rabbit IgG | anti-rabbit IgG DL755 |

ii)

| | |
|---|---|
| Ø | DAPI |
| CD68, mIgG3 | anti-mIgG3 BV480 |
| [symbol] | Ø |
| PD-1, mIgG1 | anti-mIgG1 AF546 or AF555 |
| Sox10, mIgG2b | anti-mIgG2b AF594 |
| [symbol] | Ø |
| FoxP3, rat IgG | anti-rat IgG DL755 |

iii)

| | |
|---|---|
| Ø | DAPI |
| CD68, mIgG3 | anti-mIgG3 BV480 |
| PD-1, mIgG1 | anti-mIgG1 AF488 |
| Sox10, mIgG2b | anti-mIgG2b AF546 or AF555 |
| [symbol] | Ø |
| CD3, mIgG2a | anti-mIgG2a IgG DL680 or AF647 |
| PD-L1, rabbit IgG | anti-rabbit IgG DL755 |

iv)

| | |
|---|---|
| Ø | DAPI |
| CD68, mIgG3 | anti-mIgG3 BV480 |
| PD-1, mIgG1 | anti-mIgG1 AF488 |
| Sox10, mIgG2b | anti-mIgG2b AF546 or AF555 |
| [symbol] | Ø |
| [symbol] | Ø |
| TIGIT, rabbit IgG | anti-rabbit IgG DL755 |

v)

| | |
|---|---|
| Ø | DAPI |
| CD68, mIgG3 | anti-mIgG3 BV480 |
| Sox10, mIgG2b | anti-mIgG2b AF488 |
| [symbol] | Ø |
| [symbol] | Ø |
| [symbol] | Ø |
| TIGIT, rabbit IgG | anti-rabbit IgG DL755 |

vi)

| | |
|---|---|
| Ø | DAPI |
| CD68, mIgG3 | anti-mIgG3 BV480 |
| [symbol] | Ø |
| PD-L2, mIgG2b | anti-mIgG2b AF546 or AF555 |
| [symbol] | Ø |
| [symbol] | Ø |
| PD-L1, rabbit IgG | anti-rabbit IgG DL755 |

vii)

| | |
|---|---|
| Ø | DAPI |
| CD163, mIgG2a | anti-mIgG2a BV480 |
| PD-1, mIgG1 | anti-mIgG1 AF488 |
| Sox10, mIgG2b | anti-mIgG2b AF546 or AF555 |
| [symbol] | Ø |
| FoxP3, rat IgG | anti-rat IgG DL680 or AF647 |
| PD-L1, rabbit IgG | anti-rabbit IgG DL755 |

viii)

| | |
|---|---|
| Ø | DAPI |
| CD68, mIgG3 | anti-mIgG3 BV480 |
| [symbol] | Ø |
| Sox10, mIgG2b | anti-mIgG2b AF546 or AF555 |
| [symbol] | Ø |
| TIA-1, mIgG1 | anti-mIgG1 AF647 |
| PD-L1, rabbit IgG | anti-rabbit IgG DL755 |

ix)

| Ø | DAPI |
|---|---|
| CD68, mIgG3 | anti-mIgG3 BV480 |
| Sox10, mIgG2b | anti-mIgG2b AF488 |
| [symbol] | Ø |
| [symbol] | Ø |
| TIA-1, mIgG1 | anti-mIgG1 DL680 or AF647 |
| PD-L1, rabbit IgG | anti-rabbit IgG DL755 |
| Target-binding Ab | FL |

x)

| | |
|---|---|
| Ø | DAPI |
| CD68, mIgG3 | anti-mIgG3 BV480 |
| CD3, mIgG2a | anti-mIgG2a AF488 |
| PD-1, mIgG1 | anti-mIgG1 AF546 or AF555 |
| Sox10, mIgG2b | anti-IgG2b AF594 |
| [symbol] | Ø |
| TIM-3, rabbit IgG | anti-rabbit IgG DL755 |

xi)

| | |
|---|---|
| Ø | DAPI |
| CD68, mIgG3 | anti-mIgG3 BV480 |
| CD3, mIgG2a | anti-mIgG2a AF488 |
| [symbol] | Ø |
| Sox10, mIgG2b | anti-IgG2b AF594 |
| [symbol] | Ø |
| TIM-3, rabbit IgG | anti-rabbit IgG DL755 |

xii)

| | |
|---|---|
| Ø | DAPI |
| CD68, mIgG3 | anti-mIgG3 BV480 |
| PD-1, mIgG1 | anti-mIgG1 AF488 |
| Sox10, mIgG2b | anti-mIgG2b AF546 or AF555 |
| [symbol] | Ø |
| [symbol] | Ø |
| CD31, rabbit IgG | anti-rabbit IgG DL755 |

xiii)

| | |
|---|---|
| Ø | DAPI |
| a-SMA, IgG2a | anti-mIgG2a BV480 |
| PD-1, mIgG1 | anti-mIgG1 AF488 |
| Sox10, mIgG2b | anti-mIgG2b AF546 or AF555 |
| [symbol] | Ø |
| [symbol] | Ø |
| CD31, rabbit IgG | anti-rabbit IgG DL755 |

xiv) FIGS. 16-23

| | |
|---|---|
| Ø | DAPI |
| CD68, mIgG3 | anti-mIgG3 BV480 |
| Sox10, mIgG2b | anti-mIgG2b AF488 |
| *PD-1-AF555 rabbit IgG | Ø |
| PD-L1, rabbit IgG | anti-rabbit IgG AF594 |
| CD8-AF647, mIgG1 | Ø |
| FoxP3, rat IgG | anti-rat IgG DL755 |

*Sequential staining: block with 10% rabbit serum for 10 min at RT prior
to fluorophore-conjugated 1°AB incubation
xv)

| | |
|---|---|
| Ø | DAPI |
| CD163, mIgG2a | anti-mIgG2a BV480 |
| Sox10, mIgG2b | anti-mIgG2b AF488 |
| *PD-1-AF555 rabbit IgG | Ø |
| PD-L1, rabbit IgG | anti-rabbit IgG AF594 |
| CD8-AF647, mIgG1 | Ø |
| FoxP3, rat IgG | anti-rat IgG DL755 |

*Sequential staining: block with 10% rabbit serum for 10 min at RT prior
to fluorophore-conjugated 1°AB incubation
xvi)

| | |
|---|---|
| Ø | DAPI |
| CD68, mIgG3 | anti-mIgG3 BV480 |
| Sox10, mIgG2b | anti-mIgG2b AF488 |
| PD-L1, rabbit IgG | anti-rabbit IgG AF555** |
| *CD8-AF594, mIgG1 | Ø |
| PD-1, mIgG1 | anti-mIgG1 AF647 |
| FoxP3, rat IgG | anti-rat IgG DL755 |

*Sequential staining: block with 5% mouse serum for 10 min at RT prior
to fluorophore-conjugated 1°AB incubation;
** In one embodiment AF546 is used in place of AF555
xvii)

| | |
|---|---|
| Ø | DAPI |
| CD 163, mIgG2a | anti-mIgG2a BV480 |
| Sox10, mIgG2b | anti-mIgG2b AF488 |
| PD-L1, rabbit IgG | anti-rabbit IgG AF555** |
| *CD8-AF594, mIgG1 | Ø |
| PD-1, mIgG1 | anti-mIgG1 AF647 |
| FoxP3, rat IgG | anti-rat IgG DL755 |

*Sequential staining: block with 5% mouse serum for 10 min at RT prior
to fluorophore-conjugated 1°AB incubation;
** In one embodiment AF546 is used in place of AF555
xviii)

| | |
|---|---|
| Ø | DAPI |
| CD4, mIgG2a | anti-mIgG2a BV480 |
| TIGIT, rabbit IgG | anti-rabbit IgG AF488 |
| Sox10, mIgG2b | anti-mIgG2b AF555** |
| *PD-1-AF594 rabbit IgG | Ø |
| CD8, mIgG1 | anti-mIgG1 AF647 |
| FoxP3, rat IgG | anti-rat IgG DL755 |

*Sequential staining: block with 10% rabbit serum for 10 min at RT prior
to fluorophore-conjugated 1°AB incubation;
** In one embodiment AF546 is used in place of AF555
xix)

| | |
|---|---|
| Ø | DAPI |
| CD68, mIgG3 | anti-mIgG3 BV480 |
| PD-1, mIgG1 | anti-mIgG1 AF488 |
| PRAME, mIgG2a | anti-mIgG2a AF546 or AF555 |
| [symbol] | Ø |
| FoxP3, rat IgG | anti-rat IgG DL680 or AF647 |
| PD-L1, rabbit IgG | anti-rabbit IgG DL755 |

Abbreviations:
FL—fluorophore;
mIgG1—mouse IgG1;
mIgG2a—mouse IgG2a;
mIgG2b—mouse IgG2b;
mIgG3—mouse IgG3;
CK—cytokeratin;
BV480—Brilliant Violet 480;
AF488—Alexa Fluor 488;
AF546—Alexa Fluor 546;
AF555—Alexa Fluor 555;
AF594—Alexa Fluor 594;
AF647—Alexa Fluor 647;
DL680—DyLight 680;
DL755—DyLight 680;
*bold signifies a directly-conjugated targeting antibody with the antigenic target stated first, then the fluorophore, followed by the species or isotype of the antibody.

In one embodiment at least three, four, five, six, seven, or preferably at least eight of the targeting antibody fluorophore pairs comprise antibodies of the same species and/or isotype.

In one embodiment the same species is mouse, rat, rabbit, or hamster. In one embodiment the same species is mouse. In one embodiment the same species is rat. In one embodiment the same species is rabbit. In one embodiment the species is hamster.

In one embodiment the same isotype is IgG1, IgG2, or IgG3. In one embodiment the same isotype is IgG1. In one embodiment the same isotype is IgG2. In one embodiment the same isotype is IgG3.

In another aspect the present invention relates to an antibody panel comprising at least four different targeting antibody-fluorophore pairs, wherein each targeting antibody-fluorophore pair binds a different target antigen selected from:

a T-cell related marker antigen selected from the group consisting of CD3, CD4, CD8, foxp3, T-bet, GATA-3, Granzyme B, Perforin and TIA-1,

an immune checkpoint molecule antigen selected from the group consisting of PD-1, PD-L1, PD-L2, CTLA-4, TIGIT, TIM-3, LAG-3, VISTA, CD112, CD155, Ceacam-1, Galectin-3, LSECtin, CVRL4 and PVRL4,

a tumour cell marker antigen selected from the group consisting of Sox10, S100, PRAME, Pan-CK, ER, PR, HER2 and CK8,

a myeloid cell marker antigen selected from the group consisting of CD1c, CD14, CD68, CD163, CD169 and CLEC9A, and

a stromal marker antigen selected from the group consisting of CD31, CD34, CD90, LYVE-1, a-SMA and collagen.

In one embodiment the antibody panel comprises at least five different targeting antibody fluorophore pairs. In one embodiment the antibody panel comprises at least six different targeting antibody fluorophore pairs.

In one embodiment the antibody panel comprises at least seven, eight, nine, ten, preferably eleven different targeting antibody fluorophore pairs.

In one embodiment at least one of the target antigens is a).

In one embodiment at least one of the target antigens is b).

In one embodiment at least one of the target antigens is a) and at least one of the target antigens is b).

In one embodiment at least one of the target antigens is a), at least one of the target antigens is b) and at least one of the target antigens is c), d) or e) or a combination thereof.

In one embodiment at least one of the target antigens is a), at least one of the target antigens is b) and at least one of the target antigens is c) or d) or a combination thereof.

In one embodiment at least one of the target antigens a), at least one of the target antigens b) and at least one of the target antigens c) or e) or a combination thereof.

In one embodiment at least one of the target antigens a), at least one of the target antigens b) and at least one of the target antigens d) or e) or a combination thereof.

In one embodiment at least one of the target antigens a), at least one of the target antigens is b) and at least one of each of the target antigens is c), d), and e).

In one embodiment a) is the group consisting of CD3, CD8, foxp3, and TIA-1 or a combination thereof. In one embodiment a) is CD8 or foxp3. In one embodiment a) is CD8. In one embodiment is foxp3.

In one embodiment b) is the group consisting of PD-1, PD-L1, PD-L2, TIGIT and TIM-3 or a combination thereof. In one embodiment b) is PD-1 or PD-L1 or a combination thereof. In one embodiment b) is PD-1. In one embodiment b) is PD-L1.

In one embodiment c) is the group consisting of Sox10, PRAME, Pan-CK, and CK8 or a combination thereof. In one embodiment c) is Sox10.

In one embodiment d) is CD68 or CD163 or a combination thereof. In one embodiment d) is CD163. In one embodiment d) is CD68.

In one embodiment each different targeting antibody fluorophore pair comprises an antibody selected from the group consisting of

an anti-T cell marker antibody,

an anti-immune checkpoint molecule antibody,

an anti-tumour cell marker antibody,

an anti-myeloid cell marker antibody, and

an anti-stromal marker antibody.

In one embodiment at least one of the targeting antibody fluorophore pairs comprises a). In one embodiment at least one of the targeting antibody fluorophore pairs comprises b).

In one embodiment at least one of the targeting antibody fluorophore pairs comprises a) and at least one of the targeting antibody fluorophore pairs comprises b).

In one embodiment at least one of the targeting antibody fluorophore pairs comprises a), at least one of the targeting antibody fluorophore pairs comprises b) and at least one of the targeting antibody fluorophore pairs comprises c), d) or e) or a combination thereof.

In one embodiment at least one of the targeting antibody fluorophore pairs comprises a), at least one of the targeting antibody fluorophore pairs comprises b) and at least one of the targeting antibody fluorophore pairs comprises c) or d) or a combination thereof.

In one embodiment at least one of the targeting antibody fluorophore pairs comprises a), at least one of the targeting antibody fluorophore pairs comprises b) and at least one of the targeting antibody fluorophore pairs comprises c) or e) or a combination thereof.

In one embodiment at least one of the targeting antibody fluorophore pairs comprises a), at least one of the targeting antibody fluorophore pairs comprises b) and at least one of the targeting antibody fluorophore pairs comprises d) or e) or a combination thereof.

In one embodiment at least one of the targeting antibody fluorophore pairs comprises a), at least one of the targeting antibody fluorophore pairs comprises b) and at least one of each of the targeting antibody fluorophore pairs comprises c), d), and e).

In one embodiment a) is the group consisting of anti-CD3, CD4, CD8, foxp3, T-bet, GATA-3, Granzyme B, Perforin and anti-TIA-1 antibodies or a combination thereof. In one embodiment a) is the group consisting of anti-CD3, CD8, foxp3, and anti-TIA-1 antibodies or a combination thereof. In one embodiment a) is an anti-CD8 or anti-foxp3 antibody or combination thereof.

In one embodiment b) is the group consisting of anti-PD-1, PD-L1, PD-L2, CTLA-4, TIGIT, TIM-3, LAG-3, VISTA, CD112, CD155, Ceacam-1, Galectin-3, LSECtin, CVRL4 and anti-PVRL4 antibodies or a combination thereof. In one embodiment b) is the group consisting of anti-PD-1, PD-L1, PD-L2, TIGIT and anti-TIM-3 antibodies or a combination thereof. In one embodiment b) is an anti-PD-1 or anti-PD-L1 antibody or a combination thereof.

In one embodiment c) is the group consisting of anti-Sox10, S100, PRAME, Pan-CK, ER, PR, HER2 and anti-CK8 antibodies or a combination thereof. In one embodiment c) is the group consisting of anti-Sox10, PRAME, Pan-CK, and anti-CK8 antibodies or a combination thereof. In one embodiment c) is an anti-Sox10 antibody.

In one embodiment d) is the group consisting of anti-CD1c, CD14, CD68, CD163, CD169 and anti-CLEC9A antibodies or a combination thereof. In one embodiment d) is an anti-CD68 or anti-CD163 antibody or a combination thereof. In one embodiment d) is an anti-CD68 antibody.

In one embodiment e) is the group consisting of anti-CD31, CD34, CD90, LYVE-1, alpha-Smooth Muscle Actin (a-SMA) and anti-collagen antibodies or a combination thereof. In one embodiment e) is an anti-LYVE-1 or anti-a-SMA antibody or a combination thereof.

In one embodiment a) is an anti-CD8 antibody, b) comprises anti-PD-1 and anti-PD-L1 antibodies, c) is anti-Sox10 antibody and d) is an anti-CD68 antibody.

In one embodiment a) comprises an anti-CD8 antibody and an anti-foxp3 antibody, b) comprises anti-PD-1 and anti-PD-L1 antibodies, c) is anti-Sox10 antibody and d) is an anti-CD68 antibody.

In one embodiment the targeting antibody fluorophore pairs comprising a) comprise a fluorophore selected from the group consisting of BV480, AF488, AF546, AF555, DL680, AF647, AF594, and DL755. Preferably the fluorophore is AF488, AF594, AF647, DL680 or DL755 or a combination thereof, preferably AF488, preferably AF594, preferably AF647, preferably DL680, preferably DL755.

In one embodiment the targeting antibody fluorophore pairs comprising b) comprise a fluorophore selected from the group consisting of BV480, AF488, AF546, AF555, DL680, AF647, AF594, and DL755. Preferably the fluorophore is AF488, AF555, AF647, or DL755 or a combination thereof, preferably AF488, preferably AF555, preferably AF647, preferably DL755.

In one embodiment the targeting antibody fluorophore pairs comprising c) comprise a fluorophore selected from the group consisting of BV480, AF488, AF546, AF555, DL680, AF647, AF594, and DL755. Preferably the fluorophore is AF488, AF546, AF555 or AF594, preferably AF488, preferably AF546, preferably AF555.

In one embodiment the targeting antibody fluorophore pairs comprising d) comprise a fluorophore selected from the group consisting of BV480, AF488, AF546, AF555, DL680, AF647, AF594, and DL755. Preferably the fluorophore is BV480.

In one embodiment the targeting antibody fluorophore pairs comprising e) comprise a fluorophore selected from the group consisting of BV480, AF488, AF546, AF555, DL680, AF647, AF594, and DL755. Preferably the fluorophore is DL755.

In one embodiment at least two different targeting antibody fluorophore pairs are Ab-FP conjugates. In one embodiment at least three different targeting antibody fluorophore pairs are Ab-FP conjugates. In one embodiment at least four different targeting antibody-fluorophore pairs are Ab-FP conjugates. In one embodiment at least five different targeting antibody-fluorophore pairs are Ab-FP conjugates. In one embodiment at least six different targeting antibody-fluorophore pairs are Ab-FP conjugates.

In one embodiment at least seven, eight, nine, ten, preferably eleven different targeting antibody fluorophore pairs are Ab-FP conjugates.

In one embodiment at least one Ab-FP conjugate specifically binds a target antigen selected from the group consisting of

a T-cell marker antigen,

an immune checkpoint molecule antigen,

a tumour cell marker antigen,

a myeloid cell marker antigen, and

a stromal marker antigen.

In one embodiment at least one of the target antigens is a).

In one embodiment at least one of the target antigens is b).

In one embodiment at least one of the target antigens is a) and at least one of the target antigens is b).

In one embodiment at least one of the target antigens is a), at least one of the target antigens is b) and at least one of the target antigens is selected from one of groups c), d) and e) or a combination thereof.

In one embodiment at least one of the target antigens is a), at least one of the target antigens is b) and at least one of the target antigens is c) or d) or a combination thereof.

In one embodiment at least one of the target antigens is a), at least one of the target antigens is b) and at least one of the target antigens is c) or e) or a combination thereof.

In one embodiment at least one of the target antigens is a), at least one of the target antigens is b) and at least one of the target antigens is d) or e) or a combination thereof.

In one embodiment at least one of the target antigens is a), at least one of the target antigens is b) and at least one of each of the target antigens c), or d) and e).

In one embodiment a) is selected from the group consisting of CD3, CD4, CD8, foxp3, T-bet, GATA-3, Granzyme B, Perforin and TIA-1 or a combination thereof. In one embodiment a) is selected from the group consisting of CD3, CD8, foxp3, and TIA-1 or a combination thereof. In one embodiment a) is CD8 or foxp3. In one embodiment a) is CD8. In one embodiment a) is foxp3.

In one embodiment b) is selected from the group consisting of PD-1, PD-L1, PD-L2, CTLA-4, TIGIT, TIM-3, LAG-3, VISTA, CD112, CD155, Ceacam-1, Galectin-3, LSECtin, CVRL4 and PVRL4 or a combination thereof. In one embodiment b) is selected from the group consisting of PD-1, PD-L1, PD-L2, TIGIT and TIM-3 or a combination thereof. In one embodiment b) is PD-1 or PD-L1 or a combination thereof. In one embodiment b) is PD-1. In one embodiment b) is PD-L1.

In one embodiment c) is selected from the group consisting of Sox10, S100, PRAME, Pan-CK, ER, PR, HER2 and CK8 or a combination thereof. In one embodiment c) is selected from the group consisting of Sox10, PRAME, Pan-CK, and CK8 or a combination thereof. In one embodiment c) is Sox10.

In one embodiment d) is selected from the group consisting of CD1c, CD14, CD68, CD163, CD169 and CLEC9A or a combination thereof. In one embodiment d) is CD68 or CD163 or a combination thereof. In one embodiment d) is CD163. In one embodiment d) is CD68.

In one embodiment e) is selected from the group consisting of CD31, CD34, CD90, LYVE-1, alpha-Smooth Muscle Actin (a-SMA) and collagen or a combination thereof. In one embodiment e) is LYVE-1 or a-SMA or a combination thereof. In one embodiment e) is LYVE-1. In one embodiment e) is a-SMA.

In one embodiment a) is CD8, b) comprises PD-1 and PD-L1, c) is Sox10 and d) is CD68.

In one embodiment a) comprises CD8 and foxp3, b) comprises PD-1 and PD-L1 antibodies, c) is Sox10 and d) is CD68.

In one embodiment the Ab-FP conjugates comprising a) comprise a fluorophore selected from the group consisting of BV480, AF488, AF546, AF555, DL680, AF647, AF594, and DL755. Preferably the fluorophore is AF488, AF594, AF647, DL680 or DL755 or a combination thereof, preferably AF488, preferably AF594, preferably AF647, preferably DL680, preferably DL755.

In one embodiment the Ab-FP conjugates comprising b) comprise a fluorophore selected from the group consisting of BV480, AF488, AF546, AF555, DL680, AF647, AF594, and DL755. Preferably the fluorophore is AF488, AF555, AF647, or DL755 or a combination thereof, preferably AF488, preferably AF555, preferably AF647, preferably DL755.

In one embodiment the Ab-FP conjugates comprising c) comprise a fluorophore selected from the group consisting of BV480, AF488, AF546, AF555, DL680, AF647, AF594, and DL755. Preferably the fluorophore is AF488, AF546, AF555 or AF594, preferably AF488, preferably AF546, preferably AF555.

In one embodiment the Ab-FP conjugates comprising d) comprise a fluorophore selected from the group consisting of BV480, AF488, AF546, AF555, DL680, AF647, AF594, and DL755. Preferably the fluorophore is BV480.

In one embodiment the Ab-FP conjugates comprising e) comprise a fluorophore selected from the group consisting of BV480, AF488, AF546, AF555, DL680, AF647, AF594, and DL755. Preferably the fluorophore is DL755.

Specifically contemplated embodiments of an antibody panel comprising at least four targeting antibody fluorophore pairs as described herein are set out as i) to xviii) in Table 1. Targeting antibodies from targeting antibody fluorophore pairs are shown by their antigen target and their antibody species and isotype.

In one embodiment at least two, three, four, five, six, seven, or preferably at least eight of the targeting antibody fluorophore pairs comprise antibodies of the same species and/or isotype. In one embodiment at least two or three of the targeting antibody fluorophore pairs comprise antibodies of the same species and/or isotype.

In one embodiment the same species is mouse, rat, rabbit, or hamster. In one embodiment the same species is mouse. In one embodiment the same species is rat. In one embodiment the same species is rabbit. In one embodiment the same species is a hamster.

In one embodiment the same isotype is IgG1, IgG2, or IgG3. In one embodiment the same isotype is IgG1. In one embodiment the same isotype is IgG2. In one embodiment the same isotype is IgG3.

In another aspect the invention relates to a method of determining the presence and/or abundance of a plurality of target antigens in biological sample comprising detecting in a planar sample of the biological sample,

at least a first target antigen labelled with a first targeting antibody-fluorophore pair consisting of an Ab-FP conjugate, and

at least a second target antigen labelled with a second targeting antibody-fluorophore pair,

generating a single multispectral fluorescence image of the labelled planar sample using a multispectral scanner, wherein the image comprises at least four colours, wherein each colour is associated with the specific binding of a targeting antibody to a different target antigen, and

determining from the image the presence and/or abundance of a plurality of target antigens,

wherein i) and ii) comprise antibodies of the same species and/or isotype.

In any embodiment of the above method, an antibody comprised by any targeting antibody fluorophore pair is a monoclonal antibody as described herein.

In one embodiment the method comprises a first step of labelling the at least first and second target antigens with the first and second targeting antibody fluorophore pairs respectively.

In one embodiment labelling is simultaneous or sequential. In one embodiment labelling is simultaneous and sequential.

In one embodiment the method comprises detecting at least one, two, three, four, five, six, seven, eight, or nine target antigens with at least one, two, three, four, five, six, seven, eight or nine targeting antibody fluorophore pairs respectively. Preferably the method comprises detecting at least one, two, three, four, five or six target antigens, preferably at least one, two, three, four or five, preferably at least one, two, three or four, preferably at least one, two or three, preferably at least one or two, preferably at least one target antigen with at least one, two, three, four, five or six targeting antibody fluorophore pairs, preferably at least one, two, three, four or five, preferably at least one, two, three or four, preferably at least one, two or three, preferably at least one or two, preferably at least one targeting antibody fluorophore pair, respectively.

In one embodiment the method comprises detecting four target antigens. In one embodiment the method comprises detecting five target antigens. In one embodiment the method comprises detecting six target antigens. In one embodiment the method comprises detecting seven target antigens. In one embodiment the method comprises detecting eight target antigens. In one embodiment the method comprises detecting nine target antigens. In one embodiment the method comprises detecting ten target antigens. In one embodiment the method comprises detecting eleven target antigens.

As the reader appreciates, detecting each target antigen according to the method described comprises the use of a further different targeting antibody fluorophore pair to label each further target antigen to be detected.

In one embodiment the Ab-FP conjugate in i) specifically binds a target antigen selected from

a T-cell marker antigen,

an immune checkpoint molecule antigen,

a tumour cell marker antigen,

a myeloid cell marker antigen, and

a stromal marker antigen.

In one embodiment the Ab-FP conjugate in i) specifically binds a) or b).

In one embodiment the Ab-FP conjugate in i) specifically binds a). In one embodiment the Ab-FP conjugate in i) specifically binds b). In one embodiment the Ab-FP conjugate in i) specifically binds c). In one embodiment the Ab-FP conjugate in i) specifically binds d). In one embodiment the Ab-FP conjugate in i) specifically binds e).

In one embodiment the second targeting antibody fluorophore pair specifically binds a) or b).

In one embodiment the second targeting antibody fluorophore pair specifically binds a). In one embodiment the Ab-FP conjugate in i) specifically binds b). In one embodiment the Ab-FP conjugate in i) specifically binds c). In one embodiment the Ab-FP conjugate in i) specifically binds d). In one embodiment the Ab-FP conjugate in i) specifically binds e).

In one embodiment the Ab-FP conjugate in i) specifically binds a) and the second targeting antibody fluorophore pair specifically binds b).

In one embodiment the Ab-FP conjugate in i) specifically binds b) and the second targeting antibody fluorophore pair specifically binds a).

In one embodiment the Ab-FP conjugate in i) specifically binds a) and the second targeting antibody fluorophore pair specifically binds b), wherein the method further comprises detecting at least one of c), d) or e) with at least a third targeting antibody fluorophore pair.

In one embodiment the second targeting antibody fluorophore pair is an Ab-FP conjugate.

In one embodiment the Ab-FP conjugate in i) specifically binds a) and the second targeting antibody fluorophore pair specifically binds b), wherein the method further comprises detecting at least two of c), d) or e) with at least third and fourth targeting antibody fluorophore pairs.

In one embodiment the Ab-FP conjugate in i) specifically binds a) and the second targeting antibody fluorophore pair specifically binds b), wherein the method further comprises detecting c), d) and e) with at least third, fourth and fifth targeting antibody fluorophore pairs.

In one embodiment the method comprises detecting a) or b) with a sixth targeting antibody fluorophore pair.

In some embodiments the third, fourth, fifth or sixth targeting antibody fluorophore pairs or any combination thereof are Ab-FP conjugates.

In one embodiment the Ab-FP conjugate in i) specifically binds a) and the second targeting antibody fluorophore pair specifically binds b), wherein the method further comprises detecting c) or d) or a combination thereof with at least a third targeting antibody fluorophore pair. In one embodiment the method comprises detecting c) and d) with at least third and fourth targeting antibody fluorophore pairs.

In one embodiment the Ab-FP conjugate in i) specifically binds a) and the second targeting antibody fluorophore pair specifically binds b), wherein the method further comprises detecting c) or e) or a combination thereof with at least a third targeting antibody fluorophore pair. In one embodiment the method comprises detecting c) and e) with at least third and fourth targeting antibody fluorophore pairs.

In one embodiment the Ab-FP conjugate in i) specifically binds a) and the second targeting antibody fluorophore pair specifically binds b), wherein the method further comprises detecting d) or e) or a combination thereof with at least a third targeting antibody fluorophore pair. In one embodiment the method comprises detecting d) and e) with at least third and fourth targeting antibody fluorophore pairs.

In one embodiment a) is selected from the group consisting of CD3, CD4, CD8, foxp3, T-bet, GATA-3, Granzyme B, Perforin and TIA-1 or a combination thereof. In one embodiment a) is selected from the group consisting of CD3, CD8, foxp3, and TIA-1 or a combination thereof. In one embodiment as is CD8 or foxp3. In one embodiment a) is CD8. In one embodiment a) is foxp3.

In one embodiment b) is selected from the group consisting of PD-1, PD-L1, PD-L2, CTLA-4, TIGIT, TIM-3, LAG-3, VISTA, CD112, CD155, Ceacam-1, Galectin-3, LSECtin, CVRL4 and PVRL4 or a combination thereof. In one embodiment b) is selected from the group consisting of PD-1, PD-L1, PD-L2, TIGIT and TIM-3 or a combination thereof. In one embodiment b) is PD-1 or PD-L1 or a combination thereof. In one embodiment b) is PD-1. In one embodiment b) is PD-L1.

In one embodiment c) is selected from the group consisting of Sox10, S100, PRAME, Pan-CK, ER, PR, HER2 and CK8 or a combination thereof. In one embodiment c) is selected from the group consisting of Sox10, PRAME, Pan-CK, and CK8 or a combination thereof. In one embodiment c) is Sox10.

In one embodiment d) is selected from the group consisting of CD1c, CD14, CD68, CD163, CD169 and CLEC9A or a combination thereof. In one embodiment d) is CD68 or CD163 or a combination thereof. In one embodiment d) is CD163. In one embodiment d) is CD68.

In one embodiment e) is selected from the group consisting of CD31, CD34, CD90, LYVE-1, alpha-Smooth Muscle Actin (a-SMA) and collagen or a combination thereof. In one embodiment e) is LYVE-1 or a-SMA or a combination thereof. In one embodiment e) is LYVE-1. In one embodiment e) is a-SMA.

In one embodiment a) is CD8, b) comprises PD-1 and PD-L1, c) is Sox10 and d) is CD68.

In one embodiment a) comprises CD8 and foxp3, b) comprises PD-1 and PD-L1 antibodies, c) is Sox10 and d) is CD68.

The following fluorophores are contemplated for use in targeting antibody fluorophore pairs in all of the aspects of the invention set forth herein.

In one embodiment fluorophores used in the targeting antibody fluorophore pairs described herein have maximum excitation and emission wavelengths (Ex/Em) selected from the group consisting of 348/395 nm, 404/448 nm, 405/421 nm, 405/510 nm, 405/570 nm, 405/603 nm, 405/646 nm, 405/711 nm, 407/421 nm, 415/500 nm, 436/478 nm, 490/515 nm, 494/520 nm, 495/519 nm, 485/693 nm, 496/578, 532/554 nm, 566/610 nm, 590/620 nm, 650/660 nm, 650/668 nm, 652/704, 696/719 nm, 753/785 nm, 754/787 nm, 755/775 nm and 759/775 nm.

In one embodiment, the maximum fluorescence emission wavelength (Em) of at least one, two or three of the FPs is about 710 nm to about 850 nm. In one embodiment the Em of at least one, two or three of the FPs is about 753 nm to about 759 nm, preferably of 753 nm, 754 nm, 755 nm, or 759 nm. In one embodiment the Em of one of the FPs is 754 nm.

In one embodiment the FP is selected from the group consisting of Brilliant™ Ultraviolet 395 (BUV395) having an Ex/Em of 348/395, Brilliant™ Violet 480 (BV480) having an Ex/Em of 436/478 nm, Brilliant Violet 421™ having an Ex/Em of 405/421, Brilliant™ Violet 421 (BV421) having an Ex/Em of 407/421, Brilliant™ Violet 510 (BV510) having an Ex/Em of 405/510, Brilliant Violet 570™ having an Ex/Em of 405/570, Brilliant Violet 605™ having an Ex/Em of 405/603, Brilliant Violet 650™ having an Ex/Em of 405/646, Brilliant Violet 711™ having an Ex/Em of 405/711, BD Horizon™ V450 having an Ex/Em of 404/448, BD Horizon™ V500 having an Ex/Em of 415/500, Brilliant™ Blue 515 (BB515) having an Ex/Em of 490/515 nm, Fluorescein Isothiocyanate (FITC) having an Ex/Em of 494/520 nm, Alexa Fluor 488 (AF488) having an Ex/Em of 495/519 nm, Alexa Fluor 532 (AF532) having an Ex/Em of 532/554 nm, R-phycoerythrin (PE) having an Ex/Em of 496/578, Alexa Fluor 594 (AF594) having an Ex/Em of 590/620 nm, PE-Dazzle 594 (PE594) or PE-CF594 (CF594) having an Ex/Em of 566/610 nm, Alexa Fluor 647 (AF647) having an Ex/Em of 650/668 nm, Allophycocyanin (APC) having an Ex/Em of 650/660, DyLight 680 (DL680) having an Ex/Em of 692/712 nm, BD Horizon™ 700 (BB700) having an Ex/Em of 485/693 nm, Alexa Fluor 700 (AF700) having an Ex/Em of 696/719 nm, APC/Alexa Fluor 750 having an Ex/Em of 753/785 nm, APC/Fire 750 having an Ex/Em of 754/787 nm, APC-R700 having an Ex/Em of 652/704, APC-Cy7 having an Ex/Em of 755/775 nm, DyLight 755 (DL755) having an Ex/Em of 754/776 and AF750 having an Ex/Em of 759/775 nm.

In one embodiment the fluorophore is selected from the group consisting of BV480, AF488, AF546, AF555, DL680, AF647, AF594, and DL755.

In one embodiment the Ab-FP conjugate in i) comprises, consists, or consists essentially of an antibody that specifically binds a) and a fluorophore selected from the group consisting of BV480, AF488, AF546, AF555, DL680, AF647, AF594, and DL755. Preferably the fluorophore is AF488, AF594, AF647, DL680 or DL755 or a combination thereof, preferably AF488, preferably AF594, preferably AF647, preferably DL680, preferably DL755.

In one embodiment the second targeting antibody fluorophore pair comprises, consists, or consists essentially of an antibody that specifically binds b) and a fluorophore selected from the group consisting of BV480, AF488, AF546, AF555, DL680, AF647, AF594, and DL755. Preferably the fluorophore is AF488, AF555, AF647, or DL755 or a combination thereof, preferably AF488, preferably AF555, preferably AF647, preferably DL755.

In one embodiment the method further comprises detecting a third target antigen with a third targeting antibody fluorophore pair comprising a fluorophore selected from the group consisting of BV480, AF488, AF546, AF555, DL680, AF647, AF594, and DL755. Preferably the fluorophore is AF488, AF546, AF555 or AF594, preferably AF488, preferably AF546, preferably AF555. Preferably the third target antigen is c).

In one embodiment the method further comprises detecting a fourth target antigen with a fourth targeting antibody fluorophore pair comprising a fluorophore selected from the group consisting of BV480, AF488, AF546, AF555, DL680, AF647, AF594, and DL755. Preferably the fluorophore is BV480. Preferably the fourth target antigen is d).

In one embodiment the method further comprises detecting a fifth target antigen with a targeting antibody fluorophore pair comprising a fluorophore selected from the group consisting of BV480, AF488, AF546, AF555, DL680, AF647, AF594, and DL755. Preferably the fluorophore is DL755. Preferably the fifth target antigen is e).

In some embodiments the method further comprises detecting a sixth target antigen with a targeting antibody fluorophore pair comprising a fluorophore selected from the group consisting of BV480, AF488, AF546, AF555, DL680, AF647, AF594, and DL755. Preferably the fluorophore is AF488, AF594, AF647, DL680 or DL755 or a combination thereof, preferably AF488, preferably AF594, preferably AF647, preferably DL680, preferably DL755. Preferably the sixth target antigen is a) or b).

In one embodiment at least two of the targeting antibody fluorophore pairs comprise antibodies of the same species and/or isotype. In one embodiment at least two or three of the targeting antibody fluorophore pairs comprise antibodies of the same species and/or isotype.

In one embodiment the same species is mouse, rat, rabbit, or hamster. In one embodiment the same species is mouse. In one embodiment the same species is rat. In one embodiment the same species is rabbit. In one embodiment the same species is a hamster.

In one embodiment the same isotype is IgG1, IgG2, or IgG3. In one embodiment the same isotype is IgG1. In one embodiment the same isotype is IgG2. In one embodiment the same isotype is IgG3.

In one embodiment the abundance determined in iv) is the relative abundance of at least two target antigens, preferably at least three, four, five or six target antigens.

In another aspect the invention relates to a method of determining the presence and/or abundance of a plurality of target antigens in biological sample comprising

detecting at least four target antigens in a planar sample of the biological sample, wherein each target antigen is labelled by a different targeting antibody fluorophore pair,

generating a multispectral fluorescence image of the planar sample using a multispectral scanner, wherein the image comprises at least four colours, wherein each colour is associated with the specific binding a different targeting antibody-fluorophore pair to a different target antigen, and

determining from the image the presence and abundance of the plurality of target antigens,

wherein the plurality of target antigens is selected from the group consisting of:

a T-cell related marker antigen selected from the group consisting of CD3, CD4, CD8, foxp3, T-bet, GATA-3, Granzyme B, Perforin and TIA-1,

an immune checkpoint molecule antigen selected from the group consisting of PD-1, PD-L1, PD-L2, CTLA-4, TIGIT, TIM-3, LAG-3, VISTA, CD112, CD155, Ceacam-1, Galectin-3, LSECtin, CVRL4 and PVRL4,

a tumour cell marker antigen selected from the group consisting of Sox10, S100, PRAME, Pan-CK, ER, PR, HER2 and CK8,

a myeloid cell marker antigen selected from the group consisting of CD1c, CD14, CD68, CD163, CD169 and CLEC9A, and

a stromal marker antigen selected from the group consisting of CD31, CD34, CD90, LYVE-1, a-SMA and collagen.

In one embodiment the method comprises a first step of labelling the at least four target antigens with at least four targeting antibody-fluorophore pairs respectively.

In one embodiment labelling is simultaneous or sequential. In one embodiment labelling is simultaneous and sequential.

In one embodiment the method comprises detecting at least five, six, seven, eight, or nine target antigens with at least five, six, seven, eight or nine targeting antibody fluorophore pairs respectively. Preferably the method comprises detecting at least five or six target antigens, preferably at least five, preferably at least six target antigens with at five or six targeting antibody fluorophore pairs, respectively.

In one embodiment the method comprises detecting five target antigens. In one embodiment the method comprises detecting six target antigens. In one embodiment the method comprises detecting seven target antigens. In one embodiment the method comprises detecting eight target antigens. In one embodiment the method comprises detecting nine target antigens. In one embodiment the method comprises detecting ten target antigens. In one embodiment the method comprises detecting eleven target antigens.

As the reader appreciates, detecting each target antigen according to the method described comprises the use of a different targeting antibody fluorophore pair to label each different target antigen to be detected.

In one embodiment at least one, two, three or four of the targeting antibody-fluorophore pairs in i) is an Ab-FP conjugate.

In one embodiment at least one of the targeting antibody-fluorophore pairs in i) is an Ab-FP conjugate. In one embodiment at least two of the targeting antibody-fluorophore pairs in i) are Ab-FP conjugates. In one embodiment at least three of the targeting antibody-fluorophore pairs in i) are Ab-FP conjugates. In one embodiment at least four of the targeting antibody-fluorophore pairs in i) are Ab-FP conjugates.

In one embodiment i) comprises five, six, seven, eight, nine, ten or eleven targeting antibody-fluorophore pairs that are Ab-FP conjugates.

In one embodiment the antibody in the Ab-FP conjugate is the same species and/or isotype as an antibody in at least one, preferably two, three or all of the other four targeting antibody-fluorophore pairs.

In one embodiment the antibody in the Ab-FP conjugate is the same species and/or isotype as an antibody in one, two, three or all of the other targeting antibody-fluorophore pairs.

In one embodiment at least one Ab-FP conjugate in i) specifically binds a) or b).

In one embodiment at least one Ab-FP conjugate in i) specifically binds a). In one embodiment at least one Ab-FP conjugate in i) specifically binds b). In one embodiment at least one Ab-FP conjugate in i) specifically binds c). In one embodiment at least one Ab-FP conjugate in i) specifically binds d). In one embodiment at least one Ab-FP conjugate in i) specifically binds e).

In one embodiment i) comprises at least one Ab-FP conjugate that specifically binds a) and at least one Ab-FP conjugate that specifically binds b).

In one embodiment i) comprises an Ab-FP conjugate that specifically binds b) and an Ab-FP conjugate that specifically binds a).

In one embodiment i) comprises at least one Ab-FP conjugate that specifically binds a), at least one Ab-FP conjugate that specifically binds b), and at least one Ab-FP conjugate that specifically binds c), d) or e).

In one embodiment i) comprises at least one Ab-FP conjugate that specifically binds a), at least one Ab-FP conjugate that specifically binds b), and at least one Ab-FP conjugate that specifically binds c) or d) or a combination thereof.

In one embodiment i) comprises at least one Ab-FP conjugate that specifically binds a), at least one Ab-FP conjugate that specifically binds b), and at least one Ab-FP conjugate that specifically binds c) or e) or a combination thereof.

In one embodiment i) comprises at least one Ab-FP conjugate that specifically binds a), at least one Ab-FP conjugate that specifically binds b), and at least one Ab-FP conjugate that specifically binds d) or e) or a combination thereof.

In one embodiment a) is CD8, b) comprises PD-1 and PD-L1, c) is Sox10 and d) is CD68.

In one embodiment a) comprises CD8 and foxp3, b) comprises PD-1 and PD-L1 antibodies, c) is Sox10 and d) is CD68.

Specifically contemplated as embodiments of the target antigens in a), b), c), d) and e) of this method of detection aspect of the invention are all of the specific embodiments of the target antigens set forth as a), b), c), d) and e) in the antibody panel and previous method aspects of the invention.

Additionally, specifically contemplated as embodiments of the fluorophores comprised in the targeting antibody-fluorophore pairs used in this method of detection aspect of the invention are all of the specific embodiments of fluorophores set forth in the antibody panel and previous method aspects of the invention, including all embodiments of particular fluorophores used in specified targeting antibody-fluorophore pairs.

In one embodiment at least two of the targeting antibody fluorophore pairs in i) comprise antibodies of the same species and/or isotype. In one embodiment at least two or three of the targeting antibody fluorophore pairs in i) comprise antibodies of the same species and/or isotype.

In one embodiment the same species is mouse, rat, rabbit, or hamster. In one embodiment the same species is mouse. In one embodiment the same species is rat. In one embodiment the same species is rabbit. In one embodiment the same species is a hamster.

In one embodiment the same isotype is IgG1, IgG2, or IgG3. In one embodiment the same isotype is IgG1. In one embodiment the same isotype is IgG2. In one embodiment the same isotype is IgG3.

In one embodiment the abundance determined in iii) is the relative abundance of at least two target antigens, preferably at least three, four, five or six target antigens.

In specifically contemplated embodiments, the method comprises detecting the least four target antigens in i) using the antibody panels set out as i) to xviii) in Table 1. Targeting antibodies from targeting antibody fluorophore pairs are shown by their antigen target and their antibody species and isotype.

In specifically contemplated embodiments, the detecting in i) comprises determining the presence and/or abundance of the at least four target antigens using the antibody panels set out as i) to xviii) in Table 1. Targeting antibodies from targeting antibody fluorophore pairs are shown by their antigen target and their antibody species and isotype. In one embodiment abundance is relative abundance.

In one embodiment detecting in i) comprises determining the spatial distribution of at least one target antigen based on the spatial distribution of the labelled target antigens in the sample. In one embodiment detecting in i) comprises determining the spatial distribution of at least one biomarker in the sample based on the distribution of at least one labelled target antigen. In one embodiment detecting in i) comprises determining the spatial distribution of at least one cell type in the sample based on the distribution of at least one labelled target antigen.

In another aspect the invention relates to a method of determining the presence and/or abundance of a plurality of different cell types in a biological sample comprising

detecting at least four target antigens present on or in a cell in a planar sample of the biological sample, wherein each target antigen is labelled by a different targeting antibody-fluorophore pair,

generating a single multispectral fluorescence image of the labelled planar sample using a multispectral scanner, wherein the image comprises at least five colours, wherein at least four colours are associated with the specific binding of a targeting antibody-fluorophore pair to a target antigen on a different cell type to form labelled target antigens, and

determining from the image, the presence or absence of at least four cell types based on the presence and/or abundance of the labelled target antigens,
wherein at least one of the targeting antibody fluorophore pairs is an antibody-fluorophore conjugate (Ab-FP conjugate), and
wherein at least two of the targeting antibody-fluorophore pairs comprise antibodies of the same species and/or isotype.

In one embodiment at least three targeting antibody fluorophore pairs in i) comprise antibodies of the same species and/or isotype. In one embodiment at least four, five, six, seven, eight, nine, ten, or eleven targeting antibody fluorophore pairs in i) comprise antibodies of the same species and/or isotype. Preferably from two to six targeting antibody fluorophore pairs in i) comprise antibodies of the same species and/or isotype.

In one embodiment the same species is mouse, rat, rabbit, or hamster. In one embodiment the same species is mouse. In one embodiment the same species is rat. In one embodiment the same species is rabbit. In one embodiment the same species is a hamster.

In one embodiment the same isotype is IgG1, IgG2, or IgG3. In one embodiment the same isotype is IgG1. In one embodiment the same isotype is IgG2. In one embodiment the same isotype is IgG3.

In one embodiment the method comprises a first step of labelling the at least four target antigens with at least four targeting antibody-fluorophore pairs respectively.

In one embodiment labelling is simultaneous or sequential. In one embodiment labelling is simultaneous and sequential.

In one embodiment the method comprises detecting at least five, six, seven, eight, or nine target antigens. Preferably the method comprises detecting at least five or six target antigens, preferably at least five, preferably at least six target antigens.

In one embodiment the method comprises detecting five target antigens. In one embodiment the method comprises detecting six target antigens. In one embodiment the method comprises detecting seven target antigens. In one embodiment the method comprises detecting eight target antigens. In one embodiment the method comprises detecting nine target antigens. In one embodiment the method comprises detecting ten target antigens. In one embodiment the method comprises detecting eleven target antigens.

As the reader appreciates, detecting each target antigen according to the method described comprises the use of a different targeting antibody fluorophore pair to label each different target antigen to be detected.

In one embodiment at least one, two, three or four of the targeting antibody-fluorophore pairs in i) is an Ab-FP conjugate.

In one embodiment at least one of the targeting antibody-fluorophore pairs in i) is an Ab-FP conjugate. In one embodiment at least two of the targeting antibody-fluorophore pairs in i) are Ab-FP conjugates. In one embodiment at least three of the targeting antibody-fluorophore pairs in i) are Ab-FP conjugates. In one embodiment at least four of the targeting antibody-fluorophore pairs in i) are Ab-FP conjugates.

In one embodiment i) comprises five, six, seven, eight, nine, ten or eleven targeting antibody-fluorophore pairs that are Ab-FP conjugates.

In one embodiment the antibody in the at least one Ab-FP conjugate is the same species and/or isotype as an antibody in at least one other targeting antibody-fluorophore pair.

In one embodiment the antibody in the Ab-FP conjugate is the same species and/or isotype as an antibody in one, two, three, four, five or six other targeting antibody-fluorophore pairs.

Specifically contemplated as embodiments of the target antigens in i) of this method of detecting a plurality of cell types aspect of the invention are all of the specific embodiments of the target antigens set forth as a), b), c), d) and e) in the antibody panel and previous method aspects of the invention.

Accordingly, in one embodiment at least one Ab-FP conjugate in i) specifically binds a) or b).

In one embodiment at least one Ab-FP conjugate in i) specifically binds a). In one embodiment at least one Ab-FP conjugate in i) specifically binds b). In one embodiment at least one Ab-FP conjugate in i) specifically binds c). In one embodiment at least one Ab-FP conjugate in i) specifically binds d). In one embodiment at least one Ab-FP conjugate in i) specifically binds e).

In one embodiment i) comprises at least one Ab-FP conjugate that specifically binds a) and at least one Ab-FP conjugate that specifically binds b).

In one embodiment i) comprises an Ab-FP conjugate that specifically binds b) and an Ab-FP conjugate that specifically binds a).

In one embodiment i) comprises at least one Ab-FP conjugate that specifically binds a), at least one Ab-FP conjugate that specifically binds b), and at least one Ab-FP conjugate that specifically binds c), d) or e).

In one embodiment i) comprises at least one Ab-FP conjugate that specifically binds a), at least one Ab-FP conjugate that specifically binds b), and at least one Ab-FP conjugate that specifically binds c) or d) or a combination thereof.

In one embodiment i) comprises at least one Ab-FP conjugate that specifically binds a), at least one Ab-FP conjugate that specifically binds b), and at least one Ab-FP conjugate that specifically binds c) or e) or a combination thereof.

In one embodiment i) comprises at least one Ab-FP conjugate that specifically binds a), at least one Ab-FP conjugate that specifically binds b), and at least one Ab-FP conjugate that specifically binds d) or e) or a combination thereof.

In one embodiment a) is CD8, b) comprises PD-1 and PD-L1, c) is Sox10 and d) is CD68.

In one embodiment a) comprises CD8 and foxp3, b) comprises PD-1 and PD-L1 antibodies, c) is Sox10 and d) is CD68.

Specifically contemplated as embodiments of the fluorophores comprised in the targeting antibody-fluorophore pairs used in this method of detecting a plurality of cell types aspect of the invention are all of the specific embodiments of fluorophores set forth in the antibody panel and previous method aspects of the invention, including all embodiments of particular fluorophores used in specified targeting antibody-fluorophore pairs.

In specifically contemplated embodiments, the method comprises detecting the least four target antigens in i) using the antibody panels set out as i) to xviii) in Table 1. Targeting antibodies from targeting antibody fluorophore pairs are shown by their antigen target and their antibody species and isotype.

In specifically contemplated embodiments, the detecting in i) comprises determining the presence and/or abundance of the at least four target antigens using the antibody panels set out as i) to xviii) in Table 1. Targeting antibodies from targeting antibody fluorophore pairs are shown by their antigen target and their antibody species and isotype. In one embodiment abundance is relative abundance.

In one embodiment detecting in i) comprises determining the spatial distribution of a plurality of cell types in the sample based on the spatial distribution of the target antigens detected. In one embodiment detecting in i) comprises determining the spatial distribution of at least one biomarker on and/or in cells in the sample. In one embodiment detecting in i) comprises determining the spatial distribution of at least one cell type in the sample based on the spatial distribution of the labelled target antigens.

In another aspect the invention relates to a method of detecting a plurality of different cell types in a biological sample comprising

detecting at least four target antigens present on or in a cell in a planar sample of the biological sample, wherein each target antigen is labelled by a different targeting antibody-fluorophore pair,

generating a multispectral fluorescence image of the labelled planar sample using a multispectral scanner, wherein the image comprises at least five colours, wherein the at least four colours are associated with the specific binding of a targeting antibody-fluorophore pair to a target antigen on a different cell type to form labelled target antigens, and

determining from the image, the presence or absence of at least four cell types based on the presence and/or abundance of the labelled target antigens,

wherein each targeting antibody-fluorophore pair specifically binds a different target antigen selected from:

a T-cell related marker antigen selected from the group consisting of CD3, CD4, CD8, foxp3, T-bet, GATA-3, Granzyme B, Perforin and TIA-1,

an immune checkpoint molecule antigen selected from the group consisting of PD-1, PD-L1, PD-L2, CTLA-4, TIGIT, TIM-3, LAG-3, VISTA, CD112, CD155, Ceacam-1, Galectin-3, LSECtin, CVRL4 and PVRL4,

a tumour cell marker antigen selected from the group consisting of Sox10, S100, PRAME, Pan-CK, ER, PR, HER2 and CK8,

a myeloid cell marker antigen selected from the group consisting of CD1c, CD14, CD68, CD163, CD169 and CLEC9A, and

a stromal marker antigen selected from the group consisting of CD31, CD34, CD90, LYVE-1, a-SMA and collagen.

In one embodiment the method comprises a first step of labelling the at least four target antigens with at least four targeting antibody-fluorophore pairs respectively.

In one embodiment labelling is simultaneous or sequential. In one embodiment labelling is simultaneous and sequential.

In one embodiment the method comprises detecting at least five, six, seven, eight, or nine target antigens. Preferably the method comprises detecting at least five or six target antigens, preferably at least five, preferably at least six target antigens.

In one embodiment the method comprises detecting five target antigens. In one embodiment the method comprises detecting six target antigens. In one embodiment the method comprises detecting seven target antigens. In one embodiment the method comprises detecting eight target antigens. In one embodiment the method comprises detecting nine target antigens. In one embodiment the method comprises detecting ten target antigens. In one embodiment the method comprises detecting eleven target antigens.

As the reader appreciates, detecting each target antigen according to the method described comprises the use of a different targeting antibody fluorophore pair to label each different target antigen to be detected.

In one embodiment at least one, two, three or four targeting antibody-fluorophore pairs in i) is an Ab-FP conjugate.

In one embodiment at least one of the targeting antibody-fluorophore pairs in i) is an Ab-FP conjugate. In one embodiment at least two of the targeting antibody-fluorophore pairs in i) are Ab-FP conjugates. In one embodiment at least three of the targeting antibody-fluorophore pairs in i) are Ab-FP conjugates. In one embodiment at least four of the targeting antibody-fluorophore pairs in i) are Ab-FP conjugates.

In one embodiment i) comprises five, six, seven, eight, nine, ten or eleven targeting antibody-fluorophore pairs that are Ab-FP conjugates.

In one embodiment the antibody in at least one Ab-FP conjugate is the same species and/or isotype as an antibody in at least one other targeting antibody-fluorophore pair.

In one embodiment the antibody in at least one Ab-FP conjugate is the same species and/or isotype as an antibody in one, two, three, four, five or six other targeting antibody-fluorophore pairs.

in one embodiment at least one Ab-FP conjugate in i) specifically binds a) or b).

In one embodiment at least one Ab-FP conjugate in i) specifically binds a). In one embodiment at least one Ab-FP conjugate in i) specifically binds b). In one embodiment at least one Ab-FP conjugate in i) specifically binds c). In one embodiment at least one Ab-FP conjugate in i) specifically binds d). In one embodiment at least one Ab-FP conjugate in i) specifically binds e).

In one embodiment i) comprises at least one Ab-FP conjugate that specifically binds a) and at least one Ab-FP conjugate that specifically binds b).

In one embodiment i) comprises an Ab-FP conjugate that specifically binds b) and an Ab-FP conjugate that specifically binds a).

In one embodiment i) comprises at least one Ab-FP conjugate that specifically binds a), at least one Ab-FP conjugate that specifically binds b), and at least one Ab-FP conjugate that specifically binds c), d) or e).

In one embodiment i) comprises at least one Ab-FP conjugate that specifically binds a), at least one Ab-FP conjugate that specifically binds b), and at least one Ab-FP conjugate that specifically binds c) or d) or a combination thereof.

In one embodiment i) comprises at least one Ab-FP conjugate that specifically binds a), at least one Ab-FP conjugate that specifically binds b), and at least one Ab-FP conjugate that specifically binds c) or e) or a combination thereof.

In one embodiment i) comprises at least one Ab-FP conjugate that specifically binds a), at least one Ab-FP conjugate that specifically binds b), and at least one Ab-FP conjugate that specifically binds d) or e) or a combination thereof.

In one embodiment a) is CD8, b) comprises PD-1 and PD-L1, c) is Sox10 and d) is CD68.

In one embodiment a) comprises CD8 and foxp3, b) comprises PD-1 and PD-L1 antibodies, c) is Sox10 and d) is CD68.

Specifically contemplated as embodiments of the fluorophores comprised in the targeting antibody-fluorophore pairs used in this method of detecting a plurality of cell types aspect of the invention are all of the specific embodiments of fluorophores set forth in the antibody panel and previous method aspects of the invention, including all embodiments of particular fluorophores used in specified targeting antibody-fluorophore pairs as set out in the panels in Table 1, i-xviii.

Specifically contemplated as embodiments of the antibodies comprised in the targeting antibody-fluorophore pairs used in this method of detecting a plurality of cell types aspect of the invention are all of the specific embodiments of antibodies set forth in the antibody panel and previous method aspects of the invention, including all embodiments of particular fluorophores used in specified targeting antibody-fluorophore pairs as set out in the antibody panels in Table 1, i-xviii.

In specifically contemplated embodiments, the method comprises detecting the least four target antigens in i) using the antibody panels in Table 1, i-xviii. Targeting antibodies from targeting antibody fluorophore pairs are shown by their antigen target and their antibody species and isotype.

In specifically contemplated embodiments, the detecting in i) comprises determining the presence and/or abundance of the at least four target antigens using the antibody panels in Table 1, i-xviii. Targeting antibodies from targeting antibody fluorophore pairs are shown by their antigen target and their antibody species and isotype. In one embodiment abundance is relative abundance.

In one embodiment detecting in i) comprises determining the spatial distribution of a plurality of cell types in the sample based on the spatial distribution of the target antigens detected. In one embodiment detecting in i) comprises determining the spatial distribution of at least one biomarker on and/or in cells in the sample. In one embodiment detecting in i) comprises determining the spatial distribution of at least one cell type in the sample based on the spatial distribution of the labelled target antigens.

In another aspect the invention relates to a method of identifying the presence and/or abundance of a plurality of cell types in a biological sample comprising:

labelling at least four target antigens in a planar sample of the biological sample with at least four different targeting antibody-fluorophore pairs,

using a multispectral scanner to generate a multispectral image of the labelled planar sample by detecting the fluorescence emission spectra of each fluorophore from each different targeting antibody-fluorophore pair, and

determining the presence and/or abundance of a plurality of different cell types in the planar sample based on the fluorescence emission spectra detected, optionally with reference to a suitable reference control,

wherein at least one of the targeting antibody fluorophore pairs is an antibody-fluorophore conjugate (Ab-FP conjugate), and

wherein at least two of the targeting antibody-fluorophore pairs comprise antibodies of the same species and/or isotype.

In one embodiment each targeting antibody-fluorophore pair in i) specifically binds a target antigen on or in a different cell and/or cell type.

In another aspect the invention relates to a method of identifying the presence and/or abundance of a plurality of cell types in a biological sample comprising:

labelling at least four target antigens in a planar sample of the biological sample with at least four different targeting antibody-fluorophore pairs,

using a multispectral scanner to generate a single multispectral image of the labelled planar sample by detecting the fluorescence emission spectra of each fluorophore from each different targeting antibody-fluorophore pair, and

determining the presence and/or abundance of a plurality of different cell types in the planar sample based on the fluorescence emission spectra detected, optionally with reference to a suitable reference control,

wherein each targeting antibody-fluorophore pair specifically binds a different target antigen selected from:

a T-cell related marker antigen selected from the group consisting of CD3, CD4, CD8, foxp3, T-bet, GATA-3, Granzyme B, Perforin and TIA-1,

an immune checkpoint molecule antigen selected from the group consisting of PD-1, PD-L1, PD-L2, CTLA-4, TIGIT, TIM-3, LAG-3, VISTA, CD112, CD155, Ceacam-1, Galectin-3, LSECtin, CVRL4 and PVRL4,

a tumour cell marker antigen selected from the group consisting of Sox10, S100, PRAME, Pan-CK, ER, PR, HER2 and CK8,

a myeloid cell marker antigen selected from the group consisting of CD1c, CD14, CD68, CD163, CD169 and CLEC9A, and

a stromal marker antigen selected from the group consisting of CD31, CD34, CD90, LYVE-1, a-SMA and collagen.

Specifically contemplated as embodiments of these aspects of the invention related to methods of identifying the presence and/or abundance of a plurality of cell types in a biological sample are all of the embodiments set forth in the previous method aspects of determining and of identifying, the presence and/or abundance of a plurality of different cell types including embodiments related to targeting antibody-fluorophore pairs, target antibodies including embodiments related to species and/or isotype, target antigens, Ab-FP conjugates, fluorophores, biomarkers, cell types, multispectral scanner, multispectral imaging and the antibody panels in Table 1, i-xviii.

In another aspect the invention relates to a method of making an antibody panel for an immune checkpoint related disease or condition comprising:

identifying an indicative set of at least four biomarkers for the immune checkpoint disease or condition,

obtaining a candidate targeting antibody fluorophore pair for each biomarker in the indicative set,

labelling in a planar biological sample of the immune checkpoint disease or condition, the at least four biomarkers in i) using the candidate targeting antibody-fluorophore pairs in ii),

using a multispectral scanner to generate a single multispectral image comprising the fluorescence emission spectra of each fluorophore in each targeting antibody fluorophore pair in ii)

identifying in the multispectral image the presence and/or abundance of each labelled biomarker, wherein each labelled biomarker is identified in the image as a different colour associated with the fluorescence emission spectra of each fluorophore in each targeting antibody-fluorophore pair, and

selecting the candidate targeting antibody fluorophore pairs that can be identified in the image in iv) as an antibody panel for the immune checkpoint related disease or condition,

wherein at least one of the candidate targeting antibody fluorophore pairs is an Ab-FP conjugate, and

wherein at least two of the targeting antibody-fluorophore pairs in ii) comprise antibodies of the same species and/or isotype.

In one embodiment each of the at least four biomarkers comprise a target antigen selected from the group consisting of a), b), c), d) or e) in any other aspect as described herein.

In one embodiment each candidate targeting antibody-fluorophore pair in ii) specifically binds to a target antigen selected from the group consisting of a), b), c), d) or e) in any other aspect as described herein.

In one embodiment labelling comprises labelling with a targeting antibody-fluorophore pair as described herein.

In one embodiment labelling comprises labelling with a combination of targeting antibody-fluorophore pairs and Ab-FP conjugates as described herein. A skilled reader appreciates that embodiments contemplated herein encompass all of the various combinations of targeting antibody-fluorophore pairs and Ab-FP conjugates described herein including, for example one targeting antibody-fluorophore pair and five Ab-FP conjugates, or four targeting antibody-fluorophore pairs and two Ab-FP conjugates, but not limited thereto.

In one embodiment the multispectral scanner is a Vectra Polaris.

In one embodiment selecting in vi) comprises selecting a set of targeting antibody-fluorophore pairs that an antibody panel for an immune checkpoint related disease or condition.

In one embodiment the immune checkpoint related disease or condition is a cancer. In one embodiment the cancer is tumorous cancer.

In one embodiment the cancer is selected from the group consisting of melanoma, cervical carcinoma, breast carcinoma, ovarian carcinoma, hepatocellular carcinoma, esophageal squamous cell carcinoma, gastric/gastroesophageal junction adenocarcinoma, endometrial adenocarcinoma, head and neck squamous cell carcinoma, non-small cell lung cancer and urothelial carcinoma.

In one embodiment the cancer is melanoma and at least one different targeting antibody-fluorophore pair comprises an anti-Sox10 antibody, an anti-S100 antibody, and an anti-PRAME antibody each.

In one embodiment the cancer is lung cancer or ovarian cancer and at least one targeting antibody-fluorophore pair comprises an anti-Pan-CK target antibody.

In one embodiment the cancer is breast cancer and at least one different targeting antibody-fluorophore pair comprises an anti-Pan-CK ER, TR, PR, or HER2 target antibody each.

In one embodiment the cancer is a liver cancer, and at least one targeting antibody-fluorophore pair comprises a CK8 target antibody.

Specifically contemplated as embodiments of this aspect of the invention related to a method of making an antibody panel are all of the embodiments set forth in the previous aspects of the invention related to antibody panels and to methods including embodiments related to targeting antibody-fluorophore pairs, target antibodies including embodiments related to species and/or isotype, target antigens, Ab-FP conjugates, fluorophores, biomarkers, cell types, multispectral scanner, multispectral imaging and the antibody panels in Table 1, i-xviii.

In another aspect the invention relates to a method of making an antibody panel for an immune checkpoint disease or condition comprising:

identifying an indicative set of four biomarkers for the immune checkpoint disease or condition,

obtaining a candidate targeting antibody fluorophore pair for each biomarker in the indicative set,

labelling in a planar biological sample of the immune checkpoint disease or condition, the at least four biomarkers in i) using the candidate targeting antibody-fluorophore pairs in ii),

using a multispectral scanner to generate a single multispectral image comprising the fluorescence emission spectra of each fluorophore in each targeting antibody fluorophore pair in ii)

identifying in the multispectral image the presence and/or abundance of each labelled biomarker, wherein each labelled biomarker is identified in the image as a different colour associated with the fluorescence emission spectra of each fluorophore in each targeting antibody-fluorophore pair, and

selecting the candidate targeting antibody fluorophore pairs that can be identified in the image in iv) as an antibody panel for the immune checkpoint disease or condition,

wherein

at least one biomarker in i) comprises a T-cell related marker antigen selected from the group consisting of CD3, CD4, CD8, foxp3, T-bet, GATA-3, Granzyme B, Perforin and TIA-1, and

at least one biomarker in i) comprises an immune checkpoint molecule antigen selected from the group consisting of PD-1, PD-L1, PD-L2, CTLA-4, TIGIT, TIM-3, LAG-3, VISTA, CD112, CD155, Ceacam-1, Galectin-3, LSECtin, CVRL4 and PVRL4.

In one embodiment at least one biomarker in i) comprises a tumour cell marker antigen. In one embodiment the tumour cell marker antigen is selected from the group consisting of Sox10, S100, PRAME, Pan-CK, ER, PR, HER2 and CK8.

In one embodiment at least one biomarker in i) comprises a myeloid cell marker antigen. In one embodiment the myeloid cell marker antigen is selected from the group consisting of CD1c, CD14, CD68, CD163, CD169 and CLEC9A.

In one embodiment at least one biomarker in i) comprises a stromal marker antigen. In one embodiment the stromal marker antigen is selected from the group consisting of CD31, CD34, CD90, LYVE-1, a-SMA and collagen.

In one embodiment the immune checkpoint disease and/or condition is a cancer.

In one embodiment the immune checkpoint related disease or condition is a cancer. In one embodiment the cancer is tumorous cancer.

In one embodiment the cancer is selected from the group consisting of melanoma, cervical carcinoma, breast carcinoma, ovarian carcinoma, hepatocellular carcinoma, esophageal squamous cell carcinoma, gastric/gastroesophageal junction adenocarcinoma, endometrial adenocarcinoma, head and neck squamous cell carcinoma, non-small cell lung cancer and urothelial carcinoma.

In one embodiment the cancer is selected from the group consisting of melanoma, cervical carcinoma, breast carcinoma, ovarian carcinoma, hepatocellular carcinoma, esophageal squamous cell carcinoma, gastric/gastroesophageal junction adenocarcinoma, endometrial adenocarcinoma, head and neck squamous cell carcinoma, non-small cell lung cancer and urothelial carcinoma.

In one embodiment the cancer is melanoma and the biomarkers in i) comprise Sox10, S100, and PRAME antigens.

In one embodiment the cancer is lung cancer or ovarian cancer and the biomarkers in i) comprise the Pan-CK target antigen.

In one embodiment the cancer is breast cancer and the biomarkers in i) comprise Pan-CK ER, TR, PR, and HER2 target antigens.

In one embodiment the cancer is a liver cancer, and the biomarkers comprise CK8 target antigens.

Specifically contemplated as embodiments of this aspect of the invention related to a method of making an antibody panel are all of the embodiments set forth in the previous aspects of the invention related to antibody panels and to methods including embodiments related to targeting antibody-fluorophore pairs, target antibodies including embodiments related to species and/or isotype, target antigens, Ab-FP conjugates, fluorophores, biomarkers, cell types, multispectral scanner, multispectral imaging and the antibody panels in Table 1, i-xviii

In another aspect the invention relates to a method of making an iterated antibody panel comprising:

establishing a first antibody panel that detects a core set of target antigens, the first antibody panel comprising a core set of targeting antibody-fluorophore pairs comprising at least one antibody-fluorophore conjugate (Ab-FP conjugate),

identifying a second set of core target antigens

establishing a second antibody panel that detects the second set of core antigens in ii) by replacing at least one of the targeting antibody-fluorophore pairs in i) that is not an Ab-FP conjugate with a substitute targeting antibody-fluorophore pair that specifically binds to at least one target antigen in ii),

obtaining a single multispectral image of the fluorescence emission spectra of each fluorophore from the targeting antibody fluorophore pairs in iii) by detecting in a planar biological sample the core set of target antigens from ii),

identifying in the multispectral image the second set of core target antigens from ii), wherein each core target antigen in ii) is identified in the image as a different colour that is associated with the specific binding of a different targeting antibody-fluorophore pair to a target antigen, and

selecting an iterated antibody panel comprising at least one substituted targeting antibody fluorophore pair that can be identified in the image in iv) as an iterated antibody panel,

wherein

at least one of the target antigens in ii) has been specifically labelled by at least one Ab-FP conjugate, and

at least one different target antigen in ii) has been specifically labelled by at least one substitute targeting antibody-fluorophore pair from iii).

In one embodiment at least one of the core set of target antigens in i) is a T-cell related marker antigen selected from the group consisting of CD3, CD4, CD8, foxp3, T-bet, GATA-3, Granzyme B, Perforin and TIA-1, and

In one embodiment at least one of the core set of target antigens in i) is an immune checkpoint molecule antigen selected from the group consisting of PD-1, PD-L1, PD-L2, CTLA-4, TIGIT, TIM-3, LAG-3, VISTA, CD112, CD155, Ceacam-1, Galectin-3, LSECtin, CVRL4 and PVRL4.

In one embodiment at least one of the core set of target antigens in i) comprises a tumour cell marker antigen. In one embodiment the tumour cell marker antigen is selected from the group consisting of Sox10, S100, PRAME, Pan-CK, ER, PR, HER2 and CK8.

In one embodiment at least one of the core set of target antigens in i) comprises a myeloid cell marker antigen. In one embodiment the myeloid cell marker antigen is selected from the group consisting of CD1c, CD14, CD68, CD163, CD169 and CLEC9A.

In one embodiment at least one of the core set of target antigens in i) comprises a stromal marker antigen. In one embodiment the stromal marker antigen is selected from the group consisting of CD31, CD34, CD90, LYVE-1, a-SMA and collagen.

The skilled worker appreciates that the targeting antibody-fluorophore pair is iii) may be any targeting antibody-fluorophore pair as described herein, that is used based on the disclosure provided herein.

Specifically contemplated as embodiments of this aspect of the invention related to a method of making an iterated antibody panel are all of the embodiments set forth in the previous aspects of the invention related to antibody panels and to methods including embodiments related to methods of making antibody panels encompassing embodiments related to targeting antibody-fluorophore pairs, target antibodies including embodiments related to species and/or isotype, target antigens, Ab-FP conjugates, fluorophores, biomarkers, cell types, multispectral scanner, multispectral imaging and the antibody panels in Table 1, i-xviii.

In another aspect the invention relates to a method of identifying a patient sub-group from within a group of patients comprising:

detecting at least four different biomarkers in a planar biological sample from a patient with at least four targeting antibody fluorophore pairs, wherein each targeting antibody-fluorophore pair specifically binds a target antigen on a biomarker,

generating a multispectral image of the labelled planar sample section using a multispectral scanner to detect the fluorescence emission spectra of each fluorophore from each targeting antibody-fluorophore pair,

detecting the presence or abundance of each biomarker in the image generated in ii), wherein each biomarker is identified in the image as a different colour that is associated with the specific binding of a targeting antibody-fluorophore pair to a different target antigen, and

determining from the image that a patient is in a patient sub-group based on the presence and/or abundance of each biomarker,

Specifically contemplated as embodiments of this aspect of the invention related to identifying a patient sub-group are all of the embodiments set forth in the previous aspects of the invention related to antibody panels and to methods including embodiments related to methods of making and iterating antibody panels encompassing embodiments related to targeting antibody-fluorophore pairs, target antibodies including embodiments related to species and/or isotype, target antigens, Ab-FP conjugates, fluorophores, biomarkers, cell types, multispectral scanner, multispectral imaging and the antibody panels in Table 1, i-xviii.

In another aspect the invention relates to a method for predicting a treatment response of a patient to a proposed treatment for in immune checkpoint disease or condition comprising

determining the presence, abundance and/or spatial distribution of a core set of target antigens in or on the cells in a planar biological sample from the patient using a multispectral immunofluorescence detection method as herein, and

determining whether the patient will be responsive to the proposed treatment based on the presence, abundance and/or spatial distribution of the core set of target antigens in the sample, optionally in comparison to a suitable control,

wherein

at least one of the core set of target antigens has been specifically labelled by at least one Ab-FP conjugate, and

at least two of the core set of target antigens has been specifically labelled by targeting antibody-fluorophore pairs comprising antibodies of the same species and/or isotype.

Specifically contemplated as embodiments of this aspect of the invention related to a method for predicting a treatment response of a patient as described herein are all of the embodiments set forth in the previous aspects of the invention related to antibody panels and to methods including embodiments related to methods of making and iterating antibody panels and methods of identifying a patient sub-group encompassing embodiments related to targeting antibody-fluorophore pairs, target antibodies including embodiments related to species and/or isotype, target antigens, Ab-FP conjugates, fluorophores, biomarkers, cell types, multispectral scanner, multispectral imaging and the antibody panels in Table 1, i-xviii.

In another aspect the invention relates to a method for predicting a treatment response of a patient to a proposed treatment for in immune checkpoint disease or condition comprising

determining the presence, abundance and/or spatial distribution of a core set of target antigens in or on the cells in a planar biological sample from the patient using a multispectral immunofluorescence detection method as herein, and

determining whether the patient will be responsive to the proposed treatment based on the presence, abundance and/or spatial distribution of the core set of target antigens in the sample, optionally in comparison to a suitable control,

wherein at least one of the target antigens is a T-cell related marker antigen selected from the group consisting of a T-cell related marker antigen selected from the group consisting of CD3, CD4, CD8, foxp3, T-bet, GATA-3, Granzyme B, Perforin and TIA-1, and

at least one of target antigens is an immune checkpoint molecule antigen selected from the group consisting of PD-1, PD-L1, PD-L2, CTLA-4, TIGIT, TIM-3, LAG-3, VISTA, CD112, CD155, Ceacam-1, Galectin-3, LSECtin, CVRL4 and PVRL4.

Specifically contemplated as embodiments of this aspect of the invention related to a method for predicting a treatment response of a patient as described herein are all of the embodiments set forth in the previous aspects of the invention related to antibody panels and to methods including embodiments related to methods of making and iterating antibody panels and methods of identifying a patient sub-group encompassing embodiments related to targeting antibody-fluorophore pairs, target antibodies including embodiments related to species and/or isotype, target antigens, Ab-FP conjugates, fluorophores, biomarkers, cell types, multispectral scanner, multispectral imaging and the antibody panels in Table 1, i-xviii.

In another aspect the invention relates to a method for identifying a cellular response to a candidate drug comprising

contacting a planar biological sample containing a plurality of cells with the candidate drug,

determining the abundance or spatial distribution of a core set of target antigens in the sample using a multispectral immunofluorescence detection method as herein, and

determining from the abundance or spatial distribution of the core set of antigens in sample that there is a cellular response to the candidate drug, optionally by comparison to a suitable control,

wherein

at least one of the core set of target antigens has been specifically labelled by at least one Ab-FP conjugate, and

at least two of the core set of target antigens has been specifically labelled by targeting antibody-fluorophore pairs comprising antibodies of the same species and/or isotype.

In another aspect the invention relates to a method for identifying a cellular response to a candidate drug comprising

contacting a planar biological sample containing a plurality of cells with the candidate drug,

determining the abundance or spatial distribution of a core set of target antigens in the sample using a multispectral immunofluorescence detection method as herein, and

determining from the abundance or spatial distribution of the core set of antigens in sample that there is a cellular response to the candidate drug, optionally by comparison to a suitable control,

wherein at least one of the target antigens is a T-cell related marker antigen selected from the group consisting of a T-cell related marker antigen selected from the group consisting of CD3, CD4, CD8, foxp3, T-bet, GATA-3, Granzyme B, Perforin and TIA-1, and

at least one of target antigens is an immune checkpoint molecule antigen selected from the group consisting of PD-1, PD-L1, PD-L2, CTLA-4, TIGIT, TIM-3, LAG-3, VISTA, CD112, CD155, Ceacam-1, Galectin-3, LSECtin, CVRL4 and PVRL4.

Specifically contemplated as embodiments of the above aspects of the invention related to methods for identifying a cellular response to a candidate drug as described herein are all of the embodiments set forth in the previous aspects of the invention related to antibody panels and to methods including embodiments related to methods of making and iterating antibody panels, methods of identifying a patient sub-group and methods of predicting a drug response, encompassing embodiments related to targeting antibody-fluorophore pairs, target antibodies including embodiments related to species and/or isotype, target antigens, Ab-FP conjugates, fluorophores, biomarkers, cell types, multispectral scanner, multispectral imaging and the antibody panels in Table 1, i-xviii.

In another aspect the invention relates to a method of identifying a patient that will benefit from an immune checkpoint disease therapy comprising:

labelling a planar biological sample obtained from the subject with a core set of targeting antibody fluorophore pairs,

obtaining at least one digital immunofluorescence image of the labelled sample using a multispectral scanner;

extracting data associated with at least one emission spectra associated with a targeting antibody fluorophore pair in the core set,

calculating a distribution function which captures the distribution of data for the at least one emission spectra;

deriving a summary score for a patient from the distribution function;

evaluating the summary score relative to at least one reference value; selecting the subject as a candidate for a specified cancer therapy based on the summary score, and

optionally treating the subject with the specified therapy,

wherein

at least one of the core set of targeting antibody-fluorophore pairs is an Ab-FP conjugate, and

at least two targeting antibody-fluorophore pairs comprise antibodies of the same species and/or isotype.

In another aspect the invention relates to a method of identifying a patient that will benefit from an immune checkpoint disease therapy comprising:

labelling a planar biological sample obtained from the subject with a core set of targeting antibody fluorophore pairs,

obtaining at least one digital immunofluorescence image of the labelled sample using a multispectral scanner;

extracting data associated with at least one emission spectra associated with a targeting antibody fluorophore pair in the core set,

calculating a distribution function which captures the distribution of data for the at least one emission spectra;

deriving a summary score for a patient from the distribution function;

evaluating the summary score relative to at least one reference value; selecting the subject as a candidate for a specified cancer therapy based on the summary score, and

optionally treating the subject with the specified therapy,

wherein at least one of the target antigens is a T-cell related marker antigen selected from the group consisting of a T-cell related marker antigen selected from the group consisting of CD3, CD4, CD8, foxp3, T-bet, GATA-3, Granzyme B, Perforin and TIA-1, and

at least one of target antigens is an immune checkpoint molecule antigen selected from the group consisting of PD-1, PD-L1, PD-L2, CTLA-4, TIGIT, TIM-3, LAG-3, VISTA, CD112, CD155, Ceacam-1, Galectin-3, LSECtin, CVRL4 and PVRL4.

Specifically contemplated as embodiments of the above aspects of the invention related to methods for of identifying a patient that will benefit from an immune checkpoint disease therapy as described herein are all of the embodiments set forth in the previous aspects of the invention related to antibody panels and to methods including embodiments related to methods of making and iterating antibody panels, methods of identifying a patient sub-group, methods of predicting a drug response and identifying a cellular response to a candidate drug as described herein encompassing embodiments related to targeting antibody-fluorophore pairs, target antibodies including embodiments related to species and/or isotype, target antigens, Ab-FP conjugates, fluorophores, biomarkers, cell types, multispectral scanner, multispectral imaging and the antibody panels in Table 1, i-xviii.

In another aspect the invention relates to a method of detecting a plurality of biomarkers in a biological sample comprising

labelling a core set of target antigens in a planar sample of the biological sample with a core set of targeting antibody fluorophore pairs, wherein each target antigen is comprised by a biomarker in the sample, and

generating a multispectral fluorescence image of the labelled planar sample using a multispectral scanner, wherein the image comprises at least seven colours, wherein at least six colours are associated with the specific binding of each targeting antibody fluorophore pair to a target antigen, and

determining from the image, the presence or abundance of a plurality of biomarkers, each biomarker comprising a target antigen labelled with a different targeting antibody fluorophore pair,

wherein

at least one of the core set of targeting antibody-fluorophore pairs is an Ab-FP conjugate, and

at least two targeting antibody-fluorophore pairs comprise antibodies of the same species and/or isotype.

In another aspect the invention relates to a method of detecting a plurality of biomarkers in a biological sample comprising

labelling a core set of target antigens in a planar sample of the biological sample with a core set of targeting antibody fluorophore pairs, wherein each target antigen is comprised by a biomarker in the sample, and

generating a multispectral fluorescence image of the labelled planar sample using a multispectral scanner, wherein the image comprises at least seven colours, wherein at least six colours are associated with the specific binding of each targeting antibody fluorophore pair to a target antigen, and

determining from the image, the presence or abundance of a plurality of biomarkers, each biomarker comprising a target antigen labelled with a different targeting antibody fluorophore pair,

wherein at least one of the target antigens is a T-cell related marker antigen selected from the group consisting of a T-cell related marker antigen selected from the group consisting of CD3, CD4, CD8, foxp3, T-bet, GATA-3, Granzyme B, Perforin and TIA-1, and

at least one of target antigens is an immune checkpoint molecule antigen selected from the group consisting of PD-1, PD-L1, PD-L2, CTLA-4, TIGIT, TIM-3, LAG-3, VISTA, CD112, CD155, Ceacam-1, Galectin-3, LSECtin, CVRL4 and PVRL4.

In one embodiment at least one of the targeting antibody-fluorophore pairs specifically binds to a tumour cell marker antigen. In one embodiment the tumour cell marker antigen is selected from the group consisting of Sox10, S100, PRAME, Pan-CK, ER, PR, HER2 and CK8.

In one embodiment at least one of the targeting antibody-fluorophore pairs specifically binds to a myeloid cell marker antigen. In one embodiment the myeloid cell marker antigen is selected from the group consisting of CD68 and CD163.

In one embodiment at least one of the targeting antibody-fluorophore pairs specifically binds to a stromal marker antigen. In one embodiment the stromal marker antigen is selected from the group consisting of CD31 and collagen

Specifically contemplated as embodiments of the above aspects of the invention related to methods of detecting a plurality of biomarkers in a biological sample as described herein are all of the embodiments set forth in the previous aspects of the invention related to antibody panels and to methods including embodiments related to methods of making and iterating antibody panels, methods of identifying a patient sub-group, methods of predicting a drug response and for identifying a cellular response to a candidate drug as described herein, encompassing targeting antibody-fluorophore pairs, antibodies including species and/or isotype, target antigens, Ab-FP conjugates, fluorophores, biomarkers and cell types including labelling, multispectral imaging and analysis of multispectral images and the antibody panels in Table 1, i-xviii.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents; or such sources of information, in any jurisdiction, are prior art, or form part of the common general knowledge in the art.

The invention will now be illustrated in a non-limiting way by reference to the following examples.

### EXAMPLES

### Example 1

The following protocol was used to specifically detect six different target antigens in FFPE tumour tissue using targeting antibody fluorophore pairs as described herein. The following targeting antibody fluorophore pairs were used (as shown above in Table 1, panel i); FIGS. 1-8:
anti-PD-1/anti-mouse IgG1 AF488
anti-PD-L1/anti-rabbit IgG DL755
anti-Sox10/anti-mouse IgG2b AF546
anti-CD68/anti-mouse IgG3 BV480
anti-foxp3/anti-rat IgG DL680
anti-CD8-AF594

Materials and Methods

Materials & Reagents
Positively-charged slides (for tissue sections)
Coverslips
Antigen-retrieval buffers (Sodium Citrate buffer pH 6.0 or Tris-EDTA buffer pH 9.0
DAKO PAP pen
Xylene
Ethanol
dH2O
1×TBS
Humidity Chamber
Blocker: 10% human serum+0.25% casein prepared in 1×TBS
Antibody dilution buffer: 10% human serum prepared in 1×TBS
Antibodies (purchased from Invitrogen, Abcam, Biolegend, eBioscience, Roche or Cell Signalling Technology)
Unconjugated primary (“1°”) antibodies (species, isotype)
   anti-PD-1 (mouse, IgG1)
   anti-PD-L1 (rabbit, IgG)
   anti-Sox10 (mouse, IgG2b)
   anti-CD68 (mouse, IgG3)
   anti-Foxp3 (rat, IgG)
Fluorophore-conjugated primary (“1°”) antibodies (species, isotype)
   anti-CD8-AF594 (mouse, IgG1)
Fluorophore-conjugated secondary (“2°”) antibodies
   anti-mIgG3 BV480
   anti-mIgG1 AF488
   anti-mIgG2b AF546
   anti-rat IgG DL680
   anti-rabbit IgG DL755
5% mouse serum
DAPI (use at 1:2000 final dilution)
ProlongGold mounting medium

Tissue Samples

Formalin-Fixed Paraffin-Embedded (FFPE) tissue specimens from melanoma-infiltrated lymph nodes were provided by our clinical collaborators.

FFPE Tissue Staining Protocols
(1) Bake slides in an oven, temperature set at 60° C., overnight
(2) Place the slides in staining vessels (e.g., Coplin jars or staining rack) and deparaffinize the slides by immersing the slides through the following solutions.

| | |
|---|---|
| Xylene: | 3 × 10 min |
| 99% Ethanol: | 2 × 5 min |
| 90% Ethanol: | 1 × 10 min |
| 70% Ethanol: | 10 seconds |
| dH₂O: | 10 seconds |
| 10% NBF: | 1 × 10 min |
| dH₂O: | 10 seconds |

### (3) Antigen-Retrieval

Prepare 1× antigen-retrieval buffers (Tris-EDTA buffer pH 9.0)
Immerse the slides in antigen-retrieval buffer placed in Retriever 2100 and start the retrieval process (20 min heating followed by up to 2 h cooling down)
   (4) Ab staining
Wipe away liquid around sections with paper tissue
Circle sections with PAP pen to restrict the area
Block with 10% HS+0.25% casein blocker at RT in humidity chamber for 10 min
Flick off blocker over sink
Prepare unconjugated 1° Abs at optimal concentrations, mixed as “cocktail” in dilution buffer
Incubate section with unconjugated 1° Abs up to 1 hr at RT.
Wash briefly with 1×TBS once followed by three 5 min washes with 1×TBS on rocker
Prepare fluorophore conjugated 2° Ab cocktail at optimal concentrations in dilution buffer with 10% human serum
Incubate section with fluorophore conjugated 2° Ab cocktail for 30 min in humidity chamber in the dark.
Wash briefly ×1 with TBS followed by 2×15 min washes with TBS in the dark
Block by incubating with 5% mouse serum at RT for 10 min
Wash briefly ×1 with TBS followed by 5 min washes with TBS on rocker
Prepare fluorophore conjugated 10 Abs at respective concentration in dilution buffer. Incubate section with fluorophore conjugated 1° Abs for 30 min at RT
Wash briefly ×1 with TBS followed by 2×15 min washes with TBS in the dark
   (5) Nuclear stain
Incubate section with 1:2000 DAPI for 5 min at RT in humidity chamber in the dark
Wash with 1×TBS for 2 min on rocker, then with dH₂O for 2 min on rocker
   (6) Place the coverslip on the slide with mounting medium (ProlongGold)

Scanning Protocols (from Vectra Polaris User Manual 1.0.7)

(1) Turn on the Vectra Polaris Instrument and computer
(2) Launch the Vectra Polaris software
(3) Load slides into the slide carriers
(4) Load slide carriers into the slide carrier hotel for microscope slide scanning
(5) In the ‘Edit protocol’ page (Vectra Polaris software), create a protocol for imaging. Select the fluorescent mode and spatial resolution (typically ×20 magnification, also available at ×10 or ×20) for the whole slide scanning (WSS) and for multispectral imaging (MSI) of regions of interest (ROIs). Also set the exposure times for WSS and MSI and what filters to use for focusing and imaging.
(6) In the ‘Scan slide’ page (Vectra Polaris software), locate the slides to be scanned and perform the whole slide scan (WSS) using the WSS protocol created in (5)
(7) Launch the Phenochart program (PerkinElmer) to view the WSS image and select ROIs for multispectral imaging (MSI).
(8) In the ‘Scan slide’ page (Vectra Polaris software), locate the slides containing selected ROIs to be imaged in a multispectral manner. Perform multispectral imaging (MSI) of selected ROIs using the MSI protocol created in (5)
(9) After imaging selected ROIs, unmix acquired MSI images using spectral libraries built from images of single stained tissues for each Ab-FP in the InForm software (PerkinElmer). Process and analyse unmixed images in the InForm software

### Results (Example 1)

Following the methods as described herein a seven-colour multiplex immunofluorescence image was generated (six targeting antibody fluorophore pairs+DAPI).

FFPE tissue section from melanoma-infiltrated lymph node was labelled with six targeting antibody fluorophore pairs and counter-stained with DAPI. After mounting the slide, tissue section was scanned over the whole slide using a multispectral scanner (Vectra Polaris instrument) and regions of interest (ROIs) were selected and imaged multispectrally. Acquired images were unmixed in the InForm software (PerkinElmer) (FIGS. 1-7).

The ability of the methods disclosed herein to simultaneously detect the presence and abundance of a plurality of target antigens, biomarkers and cell types is elegantly illustrated in FIGS. 1 to 8 which show the distribution of different cell populations within a melanoma-infiltrated lymph node tissue section simultaneously labelled with DAPI and six targeting antibody fluorophore pairs as described herein.

In this manner the inventors demonstrate an unexpectedly advantageous, rapid, and specific multiplex detection of multiple target antigens from a single tissue section without the need for many rounds of iterative antibody staining & stripping.

In this first example provided, multiplex immunofluorescence labelling of an FFPE tissue section employs a hybrid protocol using a combination of direct and indirect labelling. In this hybrid protocol, an initial indirect labelling step comprises the addition of five primary antibodies simultaneously to label a core set of target antigens. Bound primary antibodies are subsequently labelled in a sandwich assay by the addition of five secondary antibody-fluorophore conjugates. An Ab-FP conjugate (anti-CD8-AF594) is then added to label a sixth target antigen directly. The images derived after scanning on the Vectra Polaris instrument showed that two antibodies of the mouse IgG1 isotype could be used in the protocol (one directly-conjugated, and one unconjugated) and the antigens detected by each antibody could still be clearly distinguished in the resulting images.

The skilled person will immediately appreciate the distinct technical advantages of the methods described herein as compared to known methods of multiplex immunofluorescence detection of target antigens in FFPE.

For example, known methods allow for sequential labelling of FFPE sections only, i.e., a single primary antibody added at a time.

Following from this, it is immediately apparent to the skilled person that by employing the methods described herein they can significantly reduce the overall time and effort (e.g., the number steps) required for specific detection and identification of multiple target antigens.

### Example 2

The following protocol was used to specifically detect five different target antigens in FFPE tumour tissue using targeting antibody fluorophore pairs as described herein. The following Ab-FPs were used:
anti-PD-1/anti-mouse IgG1 AF546
anti-PD-L1/anti-rabbit IgG AF647
anti-Sox10/anti-mouse IgG2b AF488
anti-CD68/anti-mouse IgG3 BV480
anti-CD8-AF594

Materials and Methods

Materials & Reagents
Positively-charged slides (for tissue sections)
Coverslips
Antigen-retrieval buffers (Sodium Citrate buffer pH 6.0 or Tris-EDTA buffer pH 9.0
DAKO PAP pen
Xylene
Ethanol
dH₂O
1×TBS
Humidity Chamber
Blocker: 10% human serum+0.25% casein prepared in 1×TBS
Antibody dilution buffer: 10% human serum prepared in 1×TBS
Antibodies (purchased from Invitrogen, Abcam, Biolegend, eBioscience, Roche or Cell Signalling Technology)
Unconjugated primary (“1°”) antibodies (species, isotype)
   anti-CD68 (mouse, IgG3)
   anti-Sox10 (mouse, IgG2b)
   anti-PD-1 (mouse, IgG1)
   anti-PD-L1 (rabbit, IgG)
Fluorophore-conjugated primary (“1°”) antibodies (species, isotype)
   anti-CD8-AF594 (mouse, IgG1)
Fluorophore-conjugated secondary (“2°”) antibodies
   anti-mIgG3 BV480
   anti-mIgG2b AF488
   anti-mIgG1 AF546
   anti-rabbit IgG AF647
5% mouse serum
DAPI (use at 1:2000 final dilution)
ProlongGold mounting medium

Tissue Samples

Formalin-Fixed Paraffin-Embedded (FFPE) tissue specimens from melanoma-infiltrated lymph nodes were provided by our clinical collaborators.

FFPE Tissue Staining Protocols

(1) Bake slides in an oven, temperature set at 60° C., overnight
(2) Place the slides in staining vessels (e.g., Coplin jars or staining rack) and deparaffinize the slides by immersing the slides through the following solutions.

| | |
|---|---|
| Xylene: | 3 × 10 min |
| 99% Ethanol: | 2 × 5 min |
| 90% Ethanol: | 1 × 10 min |
| 70% Ethanol: | 10 seconds |
| dH₂O: | 10 seconds |
| 10% NBF: | 1 × 10 min |
| dH₂O: | 10 seconds |

### (3) Antigen-Retrieval

Prepare 1× antigen-retrieval buffers (Tris-EDTA buffer pH 9.0)
Immerse the slides in antigen-retrieval buffer placed in Retriever 2100 and start the retrieval process (20 min heating followed by up to 2 h cooling down)
   (4) Ab staining
Wipe away liquid around sections with paper tissue
Circle sections with PAP pen to restrict the area
Block with 10% HS+0.25% casein blocker at RT in humidity chamber for 10 min
Flick off blocker over sink
Prepare unconjugated 1° Abs at optimal concentrations, mixed as “cocktail” in dilution buffer
Incubate section with unconjugated 1° Abs up to 1 hr at RT.
Wash briefly with 1×TBS once followed by three 5 min washes with 1×TBS on rocker
Prepare fluorophore conjugated 2° Ab cocktail at optimal concentrations in dilution buffer with 10% human serum
Incubate section with fluorophore conjugated 2° Ab cocktail for 30 min in humidity chamber in the dark.
Wash briefly ×1 with TBS followed by 2×15 min washes with TBS in the dark
Block by incubating with 5% mouse serum at RT for 10 min
Wash briefly ×1 with TBS followed by 5 min washes with TBS on rocker
Prepare fluorophore conjugated 1° Abs at respective concentration in dilution buffer. Incubate section with fluorophore conjugated 1° Abs for 30 min at RT
Wash briefly ×1 with TBS followed by 2×15 min washes with TBS in the dark
   (5) Nuclear stain
Incubate section with 1:2000 DAPI for 5 min at RT in humidity chamber in the dark
Wash with 1×TBS for 2 min on rocker, then with dH2° for 2 min on rocker
   (6) Place the coverslip on the slide with mounting medium (ProlongGold)

Scanning Protocols (from Vectra Polaris User Manual 1.0.7)

(1) Turn on the Vectra Polaris Instrument and computer
(2) Launch the Vectra Polaris software
(3) Load slides into the slide carriers
(4) Load slide carriers into the slide carrier hotel for microscope slide scanning
(5) In the ‘Edit protocol’ page (Vectra Polaris software), create a protocol for imaging. Select the fluorescent mode and spatial resolution (typically ×20 magnification, also available at ×10 or ×20) for the whole slide scanning (WSS) and for multispectral imaging (MSI) of regions of interest (ROIs). Also set the exposure times for WSS and MSI and what filters to use for focusing and imaging.
(6) In the ‘Scan slide’ page (Vectra Polaris software), locate the slides to be scanned and perform the whole slide scan (WSS) using the WSS protocol created in (5)
(7) Launch the Phenochart program (PerkinElmer) to view the WSS image and select ROIs for multispectral imaging (MSI).
(8) In the ‘Scan slide’ page (Vectra Polaris software), locate the slides containing selected ROIs to be imaged in a multispectral manner. Perform multispectral imaging (MSI) of selected ROIs using the MSI protocol created in (5)
(9) After imaging selected ROIs, unmix acquired MSI images using spectral libraries built from images of single stained tissues for each Ab-FP in the InForm software (PerkinElmer). Process and analyse unmixed images in the InForm software

### Results (Example 2)

Following the methods described herein a six-colour multiplex immunofluorescence image was generated using a hybrid protocol (five targeting antibody fluorophore pairs+DAPI), allowing the rapid and specific multiplex immunofluorescence detection of multiple target antigens without the need for many rounds of iterative antibody staining & stripping.

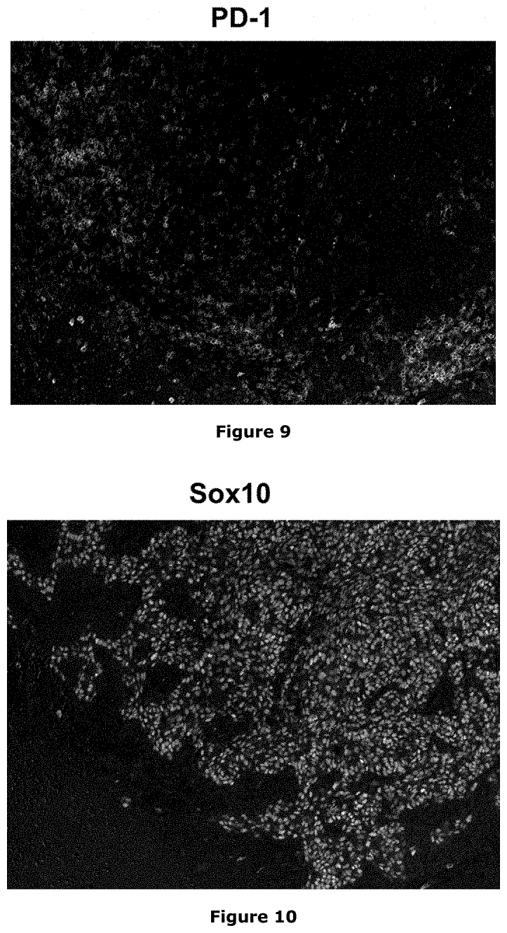

FFPE tissue section from melanoma-infiltrated lymph node was labelled with five targeting antibody fluorophore pairs and counter-stained with DAPI. After mounting the slide, tissue section was scanned over the whole slide using a multispectral scanner (Vectra Polaris instrument) and regions of interest (ROIs) were selected and imaged multispectrally. Acquired images were unmixed in the InForm software (PerkinElmer) (FIGS. 9-14).

The ability of the methods disclosed herein to simultaneously detect the presence and abundance of a plurality of target antigens, biomarkers and cell types is elegantly illustrated in FIGS. 9 to 15 which show the distribution of different cell populations within a melanoma-infiltrated lymph node tissue section simultaneously labelled with DAPI and five targeting antibody fluorophore pairs as described herein.

In this second example, four primary antibodies are added simultaneously, followed by the addition of four secondary antibodies in a sandwich type assay as described herein. Subsequently, an Ab-FP conjugate (anti-CD8-AF594) was added to the same tissue section, followed by multiplex image generation. The images showed that two antibodies of the mouse IgG1 isotype could be used in the protocol (one directly-conjugated, and one unconjugated) and the antigens detected by each antibody could still be clearly distinguished in the resulting images. Comparison of Example 2 (this example) with Example 1 also demonstrates that specific combinations of different fluorophores can be used to label the same primary antibodies, and still generate images where the antigens detected are clearly distinct. Hence certain combinations of different fluorophores are demonstrated to enable accurate imaging of a multitude of molecules within FFPE tissue sections, even when one or more of the primary antibodies is of the same species and/or isotype.

The speed and rapidity of this hybrid protocol is in contrast to known methods of generating multiplex images that employ multiple rounds of primary antibody addition including stripping and re-staining. Accordingly, this example of a method of multiplex immunofluorescence detection of a core set of target antigens as described herein illustrates a number of distinct technical advantages provided by the present disclosure including, at least, the greatly reduced overall time required for specific detection and identification of multiple target antigens.

### Example 3

The following protocol was used to specifically detect six different target antigens in FFPE tumour tissue using targeting antibody fluorophore pairs as described herein. Two different conjugated antibodies were used in this protocol, one of which had the same species/isotype to one of the unconjugated primary antibodies in the panel. The following Ab-FPs were used (as shown above in Table 1, panel xiv; FIGS. 16-23).
anti-PD-1-AF555
anti-PD-L1/anti-rabbit IgG AF594
anti-Sox10/anti-mouse IgG2b AF488
anti-CD68/anti-mouse IgG3 BV480
anti-foxp3/anti-rat IgG DL755
anti-CD8-AF647

Materials and Methods

Materials & Reagents
Positively-charged slides (for tissue sections)
Coverslips
Antigen-retrieval buffers (Sodium Citrate buffer pH 6.0 or Tris-EDTA buffer pH 9.0
DAKO PAP pen
Xylene
Ethanol
dH₂O
1×TBS
Humidity Chamber
Blocker: 10% human serum+0.25% casein prepared in 1×TBS
Antibody dilution buffer: 10% human serum prepared in 1×TBS
Antibodies (purchased from Invitrogen, Abcam, Biolegend, eBioscience, Roche or Cell Signalling Technology)
Unconjugated primary (“1°”) antibodies (species, isotype)
   anti-PD-L1 (rabbit, IgG)
   anti-CD68 (mouse, IgG3)
   anti-Sox10 (mouse, IgG2b)
   anti-foxp3 (rat, IgG)
Fluorophore-conjugated primary (“1°”) antibodies (species, isotype)
   anti-PD-1-AF555 (rabbit, IgG)
   anti-CD8-AF647 (mouse, IgG1)
Fluorophore-conjugated secondary (“2°”) antibodies
   anti-rabbit IgG AF594
   anti-mouse IgG3 BV480
   anti-mouse IgG2b AF488
   anti-rat AF546
10% rabbit serum
DAPI (use at 1:2000 final dilution)
ProlongGold mounting medium

Tissue Samples

Formalin-Fixed Paraffin-Embedded (FFPE) tissue specimens from melanoma-infiltrated lymph nodes were provided by our clinical collaborators.

FFPE Tissue Staining Protocols

(1) Bake slides in an oven, temperature set at 60° C., overnight
(2) Place the slides in staining vessels (e.g., Coplin jars or staining rack) and deparaffinize the slides by immersing the slides through the following solutions.

| | |
|---|---|
| Xylene: | 3 × 10 min |
| 99% Ethanol: | 2 × 5 min |
| 90% Ethanol: | 1 × 10 min |
| 70% Ethanol: | 10 seconds |
| dH₂O: | 10 seconds |
| 10% NBF: | 1 × 10 min |
| dH₂O: | 10 seconds |

### (3) Antigen-Retrieval

Prepare 1× antigen-retrieval buffers (Tris-EDTA buffer pH 9.0)
Immerse the slides in antigen-retrieval buffer placed in Retriever 2100 and start the retrieval process (20 min heating followed by up to 2 h cooling down)
   (4) Ab staining
Wipe away liquid around sections with paper tissue
Circle sections with PAP pen to restrict the area
Block with 10% HS+0.25% casein blocker at RT in humidity chamber for 10 min
Flick off blocker over sink
Prepare unconjugated 1° Abs and anti-CD8-AF647 at optimal concentrations, mixed as “cocktail” in dilution buffer
Incubate section with the cocktail above up to 1 hr at RT.
Wash briefly with 1×TBS once followed by three 5 min washes with 1×TBS on rocker
Prepare fluorophore conjugated 2° Ab cocktail at optimal concentrations in dilution buffer with 10% human serum
Incubate section with fluorophore conjugated 2° Ab cocktail for 30 min in humidity chamber in the dark.
Wash briefly ×1 with TBS followed by 2×15 min washes with TBS in the dark
Block by incubating with 10% rabbit serum at RT for 10 min
Wash briefly ×1 with TBS followed by 5 min washes with TBS on rocker
Prepare anti-PD-1-AF555 at respective concentration in dilution buffer. Incubate section with this Ab for 30 min at RT
Wash briefly ×1 with TBS followed by 2×15 min washes with TBS in the dark
   (5) Nuclear stain
Incubate section with 1:2000 DAPI for 5 min at RT in humidity chamber in the dark
Wash with 1×TBS for 2 min on rocker, then with dH2° for 2 min on rocker
   (6) Place the coverslip on the slide with mounting medium (ProlongGold)

Scanning Protocols (from Vectra Polaris User Manual 1.0.7)

(1) Turn on the Vectra Polaris Instrument and computer
(2) Launch the Vectra Polaris software
(3) Load slides into the slide carriers
(4) Load slide carriers into the slide carrier hotel for microscope slide scanning
(5) In the ‘Edit protocol’ page (Vectra Polaris software), create a protocol for imaging. Select the fluorescent mode and spatial resolution (typically ×20 magnification, also available at ×10 or ×20) for the whole slide scanning (WSS) and for multispectral imaging (MSI) of regions of interest (ROIs). Also set the exposure times for WSS and MSI and what filters to use for focusing and imaging.
(6) In the ‘Scan slide’ page (Vectra Polaris software), locate the slides to be scanned and perform the whole slide scan (WSS) using the WSS protocol created in (5)
(7) Launch the Phenochart program (PerkinElmer) to view the WSS image and select ROIs for multispectral imaging (MSI).
(8) In the ‘Scan slide’ page (Vectra Polaris software), locate the slides containing selected ROIs to be imaged in a multispectral manner. Perform multispectral imaging (MSI) of selected ROIs using the MSI protocol created in (5)
(9) After imaging selected ROIs, unmix acquired MSI images using spectral libraries built from images of single stained tissues for each Ab-FP in the InForm software (PerkinElmer). Process and analyse unmixed images in the InForm software

### Results (Example 3)

Following the methods described herein a seven-colour multiplex immunofluorescence image was generated using a hybrid protocol (six targeting antibody fluorophore pairs+DAPI), allowing the rapid and specific multiplex immunofluorescence detection of multiple target antigens without the need for many rounds of iterative antibody staining & stripping.

FFPE tissue section from melanoma-infiltrated lymph node was labelled with six targeting antibody fluorophore pairs and counter-stained with DAPI. After mounting the slide, tissue section was scanned over the whole slide using a multispectral scanner (Vectra Polaris instrument) and regions of interest (ROIs) were selected and imaged multispectrally. Acquired images were unmixed in the InForm software (PerkinElmer) (FIGS. 16-22).

The ability of the methods disclosed herein to simultaneously detect the presence and abundance of a plurality of target antigens, biomarkers and cell types is elegantly illustrated in FIGS. 16 to 23 which show the distribution of different cell populations within a melanoma-infiltrated lymph node tissue section simultaneously labelled with DAPI and six targeting antibody fluorophore pairs as described herein.

In this third example, four primary antibodies are added simultaneously, followed by the addition of four secondary antibodies in a sandwich type assay as described herein. Subsequently, two Ab-FP conjugates (anti-CD8-AF594 and anti-PD-1-AF555) were added to the same tissue section, followed by multiplex image generation. The images showed that two antibodies of the rabbit IgG isotype could be used in the protocol (one directly-conjugated, and one unconjugated) and the antigens detected by each antibody could still be clearly distinguished in the resulting images. Comparison of Example 3 (this example) with Example 1 also demonstrates that specific combinations of different fluorophores can be used to label the same primary antibodies, and still generate images where the antigens detected are clearly distinct. Hence certain combinations of different fluorophores are demonstrated to enable accurate imaging of a multitude of molecules within FFPE tissue sections, even when one or more of the primary antibodies is of the same species and/or isotype.

The speed and rapidity of this hybrid protocol is in contrast to known methods of generating multiplex images that employ multiple rounds of primary antibody addition including stripping and re-staining. Accordingly, this example of a method of multiplex immunofluorescence detection of a core set of target antigens as described herein illustrates a number of distinct technical advantages provided by the present disclosure including, at least, the greatly reduced overall time required for specific detection and identification of multiple target antigens.

### INDUSTRIAL APPLICATION

The targeting antibody fluorophore pairs and methods of using such of the invention have industrial application in molecular biology in providing a means to diagnose and manage immune checkpoint associated diseases and/or conditions including for theragnostic applications.

### REFERENCES

Lu, S., et al. (2019). Comparison of Biomarker Modalities for Predicting Response to PD-1/PD-L1 Checkpoint Blockade: A Systematic Review and Meta-analysis. JAMA oncology 2019.
Humphries, P., et al. (2019). Critical Appraisal of Programmed Death Ligand 1 Reflex Diagnostic Testing: Current Standards and Future Opportunities. Journal of thoracic oncology: official publication of the International Association for the Study of Lung Cancer 2019; 14(1): 45-53.
Gorris, M., et al. (2018). Eight-Color Multiplex Immunohistochemistry for Simultaneous Detection of Multiple Immune Checkpoint Molecules within the Tumor Microenvironment. J Immunol., 200(1), 347-354. doi:10.4049/jimmunol.1701262
Hofman, P., et al. (2019). Multiplexed Immunohistochemistry for Molecular and Immune Profiling in Lung Cancer—Just About Ready for Prime-Time? Cancers (Basel), 11(3):283. doi:10.3390/cancers11030283
Lichtman, J. et al. (2005). Fluorescence microscopy. Nature methods, (12): 910-9.
Tan, W. et al. (2020) Overview of multiplex immunohistochemistry/immunofluorescence techniques in the era of cancer immunotherapy. Cancer communications, 40(4): 135-53.

1. An antibody panel comprising at least four different targeting antibody-fluorophore pairs, wherein each targeting antibody-fluorophore pair binds a different target antigen selected from:
a) a T-cell related marker antigen selected from the group consisting of CD3, CD4, CD8, foxp3, T-bet, GATA-3, Granzyme B, Perforin and TIA-1,
b) an immune checkpoint molecule antigen selected from the group consisting of PD-1, PD-L1, PD-L2, CTLA-4, TIGIT, TIM-3, LAG-3, VISTA, CD112, CD155, Ceacam-1, Galectin-3, LSECtin, CVRL4 and PVRL4,
c) a tumour cell marker antigen selected from the group consisting of Sox10, S100, PRAME, Pan-CK, ER, PR, HER2 and CK8,
d) a myeloid cell marker antigen selected from the group consisting of CD1c, CD14, CD68, CD163, CD169 and CLEC9A, and
e) a stromal marker antigen selected from the group consisting of CD31, CD34, CD90, LYVE-1, a-SMA and collagen.

2. The antibody panel of claim 1, wherein the panel selected from the group of antibody panels consisting of i)-xviii) in table 1.

3. A method of determining the presence and/or abundance of a plurality of target antigens in biological sample comprising
i. detecting at least four target antigens in a planar sample of the biological sample, wherein each target antigen is labelled by a different targeting antibody fluorophore pair,
ii. generating a multispectral fluorescence image of the planar sample using a multispectral scanner, wherein the image comprises at least four colours, wherein each colour is associated with the specific binding a different targeting antibody-fluorophore pair to a different target antigen, and
iii. determining from the image the presence and abundance of the plurality of target antigens,
wherein the plurality of target antigens is selected from the group consisting of:
a) T-cell related marker antigens selected from the group consisting of CD3, CD4, CD8, foxp3, T-bet, GATA-3, Granzyme B, Perforin and TIA-1,
b) immune checkpoint molecule antigens selected from the group consisting of PD-1, PD-L1, PD-L2, CTLA-4, TIGIT, TIM-3, LAG-3, VISTA, CD112, CD155, Ceacam-1, Galectin-3, LSECtin, CVRL4 and PVRL4,
c) tumour cell marker antigens selected from the group consisting of Sox10, S100, PRAME, Pan-CK, ER, PR, HER2 and CK8,
d) myeloid cell marker antigens selected from the group consisting of CD1c, CD14, CD68, CD163, CD169 and CLEC9A, and
e) stromal marker antigens selected from the group consisting of CD31, CD34, CD90, LYVE-1, a-SMA and collagen.

4. The method of claim 3 comprising a first step of simultaneously labelling the at least four target antigens with at least four targeting antibody-fluorophore pairs respectively.

5. The method of claim 3 comprising detecting at least five, at least six or at least seven different target antigens.

6. The method of claim 3 wherein at least one, at least two, at least three, at least four, at least five, at least six or at least seven of the targeting antibody-fluorophore pairs in i) is an Ab-FP conjugate.

7. The method of claim 3 wherein a) is CD8, b) comprises PD-1 and PD-L1, c) is Sox10 and d) is CD68.

8. The method of claim 3 wherein a) comprises CD8 and foxp3, b) comprises PD-1 and PD-L1 antibodies, c) is Sox10 and d) is CD68.

9. The method of claim 3 wherein detecting in i) comprises determining the presence and/or abundance of the at least four target antigens using the antibody panels set out as i) to xviii) in Table 1.
